(19) United States
(12) Patent Application Publication
(10) Pub. No.: US 2005/0069936 A1
(43) Pub. Date: Mar. 31, 2005

(54) Diagnostic markers of depression treatment and methods of use thereof

(76) Inventors: Cornelius Diamond, San Diego, CA (US); Troy Bremer, San Diego, CA (US)

Correspondence Address: FUESS & DAVIDENAS, Suite II-G, 10951 Sorrento Valley Road, San Diego, CA, 92121-1613, US

(21) Appl. No.: 10/951,085

(22) Filed: Sep. 26, 2004

Related U.S. Application Data
(60) Provisional application No. 60/506,253, filed on Sep. 26, 2003.

(51) Int. Cl. C12Q 1/68; A61K 31/00

(52) U.S. Cl. 435/6; 514/1

(57) ABSTRACT

The present invention relates to methods for the diagnosis and evaluation of depression treatment. In particular, patient test samples are analyzed for the presence and amount of members of a panel of markers comprising one or more specific markers for depression treatment and one or more non-specific markers for depression treatment. A variety of markers are disclosed for assembling a panel of markers for such diagnosis and evaluation. Algorithms for determining proper treatment are disclosed. In various aspects, the invention provides methods for the early detection and differentiation of depression treatment. Invention methods provide rapid, sensitive and specific assays that can greatly increase the number of patients that can receive beneficial treatment and therapy, reduce the costs associated with incorrect diagnosis, and provide important information about the prognosis of the patient.

### REFERENCE TO A RELATED PROVISIONAL PATENT APPLICATION

This application is related to and claims priority from U.S. Provisional Patent Application No. 60/506,253, filed on Sep. 26, 2003, which application is hereby incorporated by reference in its entirety.

### FIELD OF THE INVENTION

The present invention relates to the identification and use of diagnostic markers for acute depression treatment. In various aspects, the invention relates to methods for the prediction of depression response to medication and the development of novel therapies in depression treatment.

### BACKGROUND OF THE INVENTION

The following discussion of the background of the invention is merely provided to aid the reader in understanding the invention and is not admitted to describe or constitute prior art to the present invention.

Major depressive disorder (MDD) affects approximately 10% of the population of the U.S. annually (NIMH 1998). The economic costs to society and personal costs to individuals and families are enormous. In a 15-month period after having been diagnosed with depression, sufferers are four times more likely to die as those who do not have depression. Almost 60% of suicides have their roots in major depression, and 15% of those admitted to a psychiatric hospital for depression eventually kill themselves (Nierenberg A A. (2001) Current perspectives on the diagnosis and treatment of major depressive disorder. Am J Manag Care 7(11 Suppl): S353-66.). In the U.S. alone, the estimated economic costs for depression exceeded $44 billion in 1990. The World Health Organization estimates that major depression is the fourth most important cause worldwide of loss in disability-adjusted life years, and will be the second most important cause by 2020 (Agency for Health Care Policy and Research R, MD. (1999).

Anti-depressants are a primary method for treatment of depression. Prescription of anti-depressant medication, however, is inexact. Not all patients receiving an anti-depressant medication will respond to that treatment. Others may respond, but with serious side effects. The period required to determine the efficacy of treatment response can be both costly and lengthy. A method for rapid identification of appropriate treatment for patients is needed. Recent research has indicated that characteristics such as age, gender, ethnicity, weight, diagnosis, and diet affect both the pharmacokinetics and pharmacodynamics of psychotropic medication (Lawson W B. (1996) The art and science of the psychopharmacotherapy of African Americans. Mt Sinai J Med 63(5-6): 301-5., Lin K, Poland R, Wan Y, Smith M, Strickland T L, Mendoza R. (1991) Pharmacokinetic and other related factors affecting psychotropic responses in Asians. Psychopharmacology. Bulletin 27(427-439., Mendoza R, Smith M W, Poland R E, Lin K M, Strickland T L. (1991) Ethnic psychopharmacology: the Hispanic and Native American perspective. Psychopharmacol Bull 27(4): 449-61., Roberts J, Tumer N. (1988) Pharmacodynamic basis for altered drug action in the elderly. Clin Geriatr Med 4(1): 127-49. Rosenblat R, Tang S. (1987) Do Oriental psychiatric patients receive different dosages of psychotropic medication when compared with Occidentals? Can. J. Psychiatry 32(270-274., Strickland T L, Ranganath V, Lin K M, Poland R E, Mendoza R, Smith M W. (1991) Psychopharmacologic considerations in the treatment of black American populations. Psychopharmacol Bull 27(4): 441-8.,Dawkins K, Pofter W Z. (1991) Gender differences in pharmacokinetics and pharmacodynamics of psychotropics: focus on women. Psychopharmacol Bull 27(4): 417-26.). However, no method currently exists for incorporating these variables into a predictive algorithm for prescribing medication. Recently, attention has focused on the identification of Single Nucleotide Polymorphisms, (hereafter SNPs) as factors that specifically influence drug action or act as markers for alleles of genes that influence drug action (Xu J, Zheng S L, Hawkins G A, Faith D A, Kelly B, Isaacs S D, Wiley K E, Chang B, Ewing C M, Bujnovszky P, Carpten J D, Bleecker E R, Walsh P C, Trent J M, Meyers D A, Isaacs W B. (2001) Linkage and association studies of prostate cancer susceptibility: evidence for linkage at 8p22-23. Am J Hum Genet 69(2): 341-50.), These are important clinical markers. Incorporation of these markers (in conjunction patient chart data) into a predictive algorithm will allow accurate, personalized prescription of anti-depressant medication.

As an independent variable, either a SNP or a patient characteristic is unlikely, itself, to indicate a responder phenotype with acceptable confidence—a direct causal effect on phenotype is rare. However, understanding the complex interactions that result in a response phenotype for more than a small number of variables are not realistic without comprehensive analysis technology. This patent will show how to use such analysis algorithms that have the ability to extract meaningful information from complex interactions occurring between multiple variables.

In recent years, the search for a single gene responsible for major depressive disorder has given way to the understanding that multiple gene variants, acting together with yet unknown environmental risk factors or developmental events, interact in a complex system to account for its expression phenotype. In accordance, treatments that successfully alleviate depression symptoms are likely to act on multiple gene products.

Assessing Patient Response to Depression Treatment

Responder/non-responder phenotypes of treatment efficacy are determined quantitatively by one or more rating scales, the most popular being the Hamilton Rating Scale for Depression (HAM-D), Emotional State Questionaire or Global Clinical Impression Scale, the subsection of relevance being Improvement (CGI-I). The HAM-D scale, first published in 1960 and since revised, contains items that assess somatic symptoms, insomnia, working capacity and interest, mood, guilt, psychomotor retardation, agitation, anxiety, and insight. The HAM-D offers high validity and reliability in measuring response to treatment (Marder S, Psychiatric rating scales. S. B. Kaplan H I, Ed., Comprehensive Textbook of Psychiatry/VI 6th ed. (Williams & Wilkins, Baltimore, Md., 1995), vol. 1.). The maximum possible score for the 21-item HAM-D is 52; in practice, very few patients score above 35. Most people with depression score 14 or more. Scores of 30 or higher are more typical of severely depressed patients.

As an independent measure, a ≧50% decrease in HAM-D score may be considered a response (Lecrubier Y, Clerc G, Didi R, Kieser M. Related Articles, Links Abstract Efficacy of St. John's wort extract WS 5570 in major depression: a double-blind, placebo-controlled trial. Am J Psychiatry. August 2002;159(8):1361-6.). However, in this patent it is found that using three different measures of emotional state that were averaged would provide a more reliable assessment of response than the HAM-D test alone.

While the Dep section of the Emotional State questionnaire and HAM-D are scored both before and after treatment to obtain change in score, the final CGI-I is a single score (from 1 to 7) given by the doctor at the end of the study assessing the patient's improvement. In order to take an average of all three scores it is necessary to scale the CGI-I test score to that of both the HAM-D and Dep scores.

The CGI-I test has the following structure:

| | |
|---|---|
| Very much improved | □1 |
| Much improved | □2 |
| Minimally improved | □3 |
| No change | □4 |
| Minimally worse | □5 |
| Much worse | □6 |
| Very much worse | □7 |

Current diagnostic methods for depression treatment are basically trial-and-error. A person is given a medication at usually a low dosage, then titrated upwards in dosage over a period of weeks or months. After several months, the person is evaluated again by a physician to determine if the person's depression level has changed and/or an adverse event is registered. If it has not changed enough in a positive direction to suit the patient and/or physician, the person is gradually titrated downwards on the first drug and the process repeats itself with another medication. It is not uncommon for a patient to repeat this process over a period of years, all the while suffering physically, emotionally, and financially.

Accordingly, there is a present need in the art for a rapid, sensitive and specific diagnostic assay for depression treatment that can also differentiate the type of medication and identify those individuals at risk for adverse events. Such a diagnostic assay would greatly increase the number of patients that can receive beneficial treatment and therapy, and reduce the costs associated with incorrect therapy.

### SUMMARY OF THE INVENTION

The present invention relates to the identification and use of diagnostic and/or prognostic markers for psychotropics, anti-depressants, Selective Serotonin Reuptake Inhibitors, and/or the anti-depressant citalopram and paroxetine. The methods and compositions described herein can meet the need in the art for a rapid, sensitive and specific diagnostic assay to be used to facilitate the treatment of depression patients and the development of additional diagnostic indicators. Moreover, the methods and compositions of the present invention can also be used in diagnosis, differentiation and prognosis of various forms of psychotropic disorders.

The terms “psychotropic disorder and psychotropics” relate to the diseases of depression, bipolar disorder, schizophrenia, other depressive disorders and the pharmaceutical agents used to treat them, respectively. One skilled in the art will recognize these terms, which are described in “The Merck Manual of Diagnosis and Therapy” Seventeenth Edition, 1999, Ed. Keryn A. G. Lane, pp. 1503-1598, incorporated by reference only. In various aspects, the invention relates to materials and procedures for identifying markers that are associated with the diagnosis, prognosis, or differentiation of depression treatment in a patient; to using such markers in diagnosing and treating a patient and/or to monitor the course of a treatment regimen; and for screening compounds and pharmaceutical compositions that might provide a benefit in treating or preventing such conditions.

In a first aspect, the invention features methods of diagnosing depression by analyzing a test sample obtained from a patient for the presence or amount of one or more SNPs associated with genes in the serotonin, adsorption, distribution, receptor or effector biochemical pathways. These methods can include identifying one or more SNPs, the presence or amount of which is associated with the treatment, diagnosis, prognosis, or differentiation of depression. Once such SNP(s) are identified, the pattern of such SNPs in a patient sample can be measured. In certain embodiments, these markers can be compared to a diagnostic level determined by an algorithm that is associated with the treatment, diagnosis, prognosis, or differentiation of depression. By correlating the patient pattern to the diagnostic pattern, the presence or absence of depression, and the probability of treatment outcomes in a patient may be rapidly and accurately determined.

For purposes of the following discussion, the methods described as applicable to the treatment outcome and diagnosis of depression treatment generally may be considered applicable to the treatment outcome and diagnosis of the depressive phase of bipolar disorder and other depressive disorders such as anxiety and seasonal affective disorder.

In certain embodiments, a plurality of SNPs are combined to increase the predictive value of the analysis in comparison to that obtained from the markers individually or in smaller groups. Preferably, one or more specific markers for depression treatment can be combined with one or more non-specific markers for depression treatment to enhance the predictive value of the described methods.

To date, SNPs and various proteins have not been used as markers of depression or other psychotropic disorders. Additionally, other markers of various pathological processes including serotonin, dopamine, or norepinephrine transport protein have not been used as subsets of a larger panel of markers of depression. Preferred markers of the invention can aid in the treatment, diagnosis, differentiation, and prognosis of patients with depression, bipolar disorder, and schizophrenia.

The term “test sample” as used herein refers to a biological sample obtained for the purpose of diagnosis, prognosis, or evaluation. In certain embodiments, such a sample may be obtained for the purpose of determining the outcome of an ongoing condition or the effect of a treatment regimen on a condition. Preferred test samples include blood, serum, plasma, cerebrospinal fluid, urine and saliva. In addition, one of skill in the art would realize that some test samples would be more readily analyzed following a fractionation or purification procedure, for example, separation of whole blood into serum or plasma components.

The term “specific marker of depression treatment” as used herein refers to SNPs that are typically associated with psychotropic disorders, and which can be correlated with depression, but are not correlated with other types of disease. Such specific SNPs of depression include those involved in preferential inhibition of the serotonin transport protein (resulting in increases in synaptic levels of serotonin with resultant serotonin autoreceptor desensitization), the norepinephrine transport protein (NET) and those involved in dopamine receptor sensitivity. These systems, and others proposed to be involved in depression and affected by specific drugs (e.g. HPA axis [Pitchot, 2001 #17]), are in certain embodiments of the invention are candidates for gene/SNP sets to be used as system inputs for a predictive algorithm. These specific markers are described in detail hereinafter.

The term “non-specific marker of SSRI therapeutic action” as used herein refers to molecules that are typically general markers of therapeutic SSRI response. Such markers may be present in the event of SSRI response, but may also be present in general depressives. Factors including genetic variants of the serotonin transporter, serotonin-2A-receptor, tryptophan hydroxylase, brain-derived neurotrophic factor, G-protein beta3 subunit, interleukin-1beta and angiotensin-converting enzyme. These non-specific markers are described in detail hereinafter.

Other non-specific markers of depression include markers of CREB's participation in antidepressant response, as well as BDNF trophic effect and their intracellular signaling pathways, corticotrophin releasing factor receptors and G beta 3 variants.

The skilled artisan will recognize that nucleotide position can be found from reference sequence number (hereafter RS#) information by referring to a public database such as www.snpper.chip.org.

The phrase “diagnosis” as used herein refers to methods by which the skilled artisan can estimate and even determine whether or not a patient is suffering from a given disease or condition. The skilled artisan often makes a diagnosis on the basis of one or more diagnostic indicators, i.e., a marker, the presence, absence, or amount of which is indicative of the presence, severity, or absence of the condition.

Similarly, a prognosis is often determined by examining one or more “prognostic indicators.” These are markers, the presence or amount of which in a patient (or a sample obtained from the patient) signal a probability that a given course or outcome, including treatment outcome, will occur. For example, when one or more prognostic indicators exhibit a certain pattern or level in samples obtained from such patients, the pattern or level may signal that the patient is at an increased probability for experiencing a future event in comparison to a similar patient exhibiting a different pattern or lower marker level. A certain pattern, level or a change in level of a prognostic indicator, which in turn is associated with an increased probability of disease recurrence or side effect such as obesity, is referred to as being “associated with an increased predisposition to an adverse outcome” in a patient. Preferred prognostic markers can predict the onset of delayed adverse events in a patient, or the chance of a person responding or not responding to a certain drug.

The term “correlating,” as used herein in reference to the use of diagnostic and prognostic indicators, refers to comparing the presence or amount of the indicator in a patient to its presence or amount in persons known to respond to a certain treatment; suffer from, or known to be at risk of, a given condition; or in persons known to be free of a given condition, i.e. “normal individuals”. For example, a SNP pattern or marker level in a patient sample can be compared to a SNP pattern or level known to be associated with response to a certain depression medication. The sample's marker pattern or level is said to have been correlated with a diagnosis; that is, the skilled artisan can use the marker pattern or level to determine whether the patient will respond to a certain medication, and prescribe accordingly. Alternatively, the sample's SNP pattern or marker level can be compared to a SNP pattern or marker level known to be associated with an adverse event (e.g., tardive diskinesa), such as an SNP pattern or average level found in a population of normal individuals.

In certain embodiments, a diagnostic or prognostic indicator is correlated to a condition or disease by merely its presence or absence. In other embodiments, an algorithm is needed to relate the pattern of markers to a desired prediction outcome in the patient. A preferred algorithmic technique for relating markers of the present invention is a linear regression technique, a nonlinear regression technique, an ANOVA technique, a neural network technique, a genetic algorithm technique, a support vector machine technique, a tree learning technique, a nonparametric statistical technique, a forward, backward, and/or forward-backward technique, and a Bayesian technique. The skilled artisan will recognize the word “technique” refers to a process in which a predictor is built by using patient exemplar pairs of markers and phenotypes, and then refining such predictor algorithm in an iterative process by testing a version of the algorithm on unseen data and making changes to mathematical coefficients of such algorithm in such a way to increase the accuracy and specificity of the predictor algorithm.

In other embodiments, the invention relates to methods for determining a treatment regimen for use in a patient diagnosed with depression, particularly for the SSRI citalopram. The methods preferably comprise determining a level of one or more diagnostic or prognostic markers as described herein, and using the markers to determine a diagnosis for a patient. One or more treatment regimens that improve the patient's prognosis by reducing the increased disposition for an adverse outcome associated with the diagnosis can then be used to treat the patient. Such methods may also be used to screen pharmacological compounds for agents capable of improving the patient's prognosis as above.

In yet another embodiment, multiple determination of one or more diagnostic or prognostic markers can be made, and a temporal change in the marker can be used to monitor the efficacy of appropriate therapies. In such an embodiment, one might expect to see a decrease or an increase in the marker(s) over time during the course of effective therapy.

The skilled artisan will understand that, while in certain embodiments comparative measurements are made of the same diagnostic marker at multiple time points, one could also measure a given marker at one time point, and a second marker at a second time point, and a comparison of these markers may provide diagnostic information. The skilled artisan will also understand that proteomic or gene expression values may change in time, SNP patterns by definition are fixed in time.

The phrase “determining the prognosis” as used herein refers to methods by which the skilled artisan can predict the course or outcome of a condition in a patient. The term “prognosis” does not refer to the ability to predict the course or outcome of a condition with 100% accuracy, or even that a given course or outcome is predictably more or less likely to occur based on the presence, absence or levels of test markers. Instead, the skilled artisan will understand that the term “prognosis” refers to an increased probability that a certain course or outcome will occur; that is, that a course or outcome is more likely to occur in a patient exhibiting a given condition, such as nicotine dependence, when compared to those individuals not exhibiting the condition.

The skilled artisan will understand that associating a prognostic indicator with a predisposition to an adverse outcome is a statistical analysis. For example, a marker level of greater than 80 pg/mL may signal that a patient is more likely to suffer from an adverse outcome than patients with a level less than or equal to 80 pg/mL, as determined by a level of statistical significance. Additionally, a change in marker concentration from baseline levels may be reflective of patient prognosis, and the degree of change in marker level may be related to the severity of adverse events. Statistical significance is often determined by comparing two or more populations, and determining a confidence interval and/or a p value. See, e.g., Dowdy and Wearden, Statistics for Research, John Wiley & Sons, New York, 1983. Preferred confidence intervals of the invention are 90%, 95%, 97.5%, 98%, 99%, 99.5%, 99.9% and 99.99%, while preferred p values are 0.1, 0.05, 0.025, 0.02, 0.01, 0.005, 0.001, and 0.0001. Exemplary statistical tests and algorithmic methods for associating a prognostic indicator with a predisposition to an adverse outcome and success or failure on a treatment regime are described hereinafter.

In yet other embodiments, multiple determination of one or more diagnostic or prognostic markers can be made, and a temporal change in the marker can be used to determine a diagnosis or prognosis. For example, a diagnostic indicator may be determined at an initial time, and again at a second time. In such embodiments, an increase in the marker from the initial time to the second time may be diagnostic of a particular type of depression, such as treatment-resistant depression, or a given prognosis. Likewise, a decrease in the marker from the initial time to the second time may be indicative of a particular type of depression, or a given prognosis. Furthermore, the degree of change of one or more markers may be related to the severity of the disease and future adverse events.

In a further aspect, the invention relates to kits for determining the diagnosis or prognosis of a patient. These kits preferably comprise devices and reagents for measuring one or more SNP patterns or marker levels in a patient sample, and instructions for performing the assay. Optionally, the kits may contain one or more means for converting SNP patterns or marker level(s) to a prognosis. Such kits preferably contain sufficient reagents to perform one or more such determinations.

### DETAILED DESCRIPTION OF THE INVENTION

In accordance with the present invention, there are provided methods and compositions for the identification and use of markers that are associated with the diagnosis, prognosis, or differentiation of depression in a patient. Such markers can be used in diagnosing and treating a patient and/or to monitor the course of a treatment regimen; and for screening compounds and pharmaceutical compositions that might provide a benefit in treating or preventing such conditions.

Depression is a common, life-disrupting, potentially lethal illness that can affect both sexes and all ages. Its peak onset is in the early adult years. It is more common than hypertension in primary care practice. Recent studies show that fewer than 1 in 20 depressed patients are correctly diagnosed and adequately treated. Depression periodically destroys the productivity of those with the condition, and depressed patients have a worse quality of life than patients with debilitating, chronic conditions such as arthritis, hypertension, diabetes mellitus and back pain. Suicide occurs in as many as 15% of patients with depression, especially those with recurrent episodes and hospitalisations, and may even occur in those with in subsyndromal depression.

In recent years, the search for a single gene responsible for major depressive disorder has given way to the understanding that multiple gene variants, acting together with yet unknown environmental risk factors or developmental events, interact in a complex system to account for its expression phenotype. In accordance, treatments that successfully alleviate depression symptoms are likely to act on multiple gene products.

Selective serotonin (5-hydroxytryptamine; 5-HT) reuptake inhibitors (SSRIs) are the cornerstone of modern pharmacotherapy for effective treatment of depression. Prior to the SSRIs, all psychotropic medications were the result of chance observation. In an attempt to develop a SSRI, researchers discovered a number of nontricyclic agents with amine-uptake inhibitory properties, acting on both noradrenergic and serotonergic neurons with considerable differences in potency. A given drug may affect one or more sites over its clinically relevant dosing range and may produce multiple and different clinical effects. The enhanced safety profile includes a reduced likelihood of pharmacodynamically mediated adverse drug-drug interactions by avoiding affects on sites that are not essential to the intended outcome. SSRIs were developed for inhibition of the neuronal uptake pump for serotonin (5-HT), a property shared with the TCAs, but without affecting the other various neuroreceptors or fast sodium channels. The therapeutic mechanism of action of SSRIs involves alteration in the 5-HT system. The plethora of biological substrates, receptors and pathways for 5-HT are candidates to mediate not only the therapeutic actions of SSRIs, but also their side effects.

As they are well tolerated, even in the presence of comorbid medical illness, and easier to manage, SSRIs enhance compliance. A fully adequate antidepressant dosage is suitable for patients of all ages and can be used by non-psychiatrist physicians for the treatment of the acute episode, as well as the frequent recurrences that often require long term maintenance antidepressant medication. SSRIs have fewer drug interactions than older antidepressants, and even the SSRI inhibition of hepatic cytochrome P450 enzymes has proven only very infrequently to be of clinical importance. SSRIs also effectively treat anxious depression, dysthymia and atypical depression.

Citalopram is an SSRI antidepressant that is highly selective for the serotonin transport protein. It has negligible, if any, interaction with dopamine and/or norepinephrine transporters. It is well tolerated, and drug interactions are not a significant concern. It is also reasonably safe for populations vulnerable to pharmacokinetic effects, such as the elderly and patients with metabolic diseases (Bezchlibnyk-Butler K, Aleksic I, Kennedy S H. (2000) Citalopram—a review of pharmacological and clinical effects. J Psychiatry Neurosci 25(3): 241-54.).

An important aspect of determining response is metabolic capacity for the drug. Citalopram is primarily metabolized by CYP3A4 and CYP2D19. The metabolites of this reaction are further metabolized by CYP2D6 (see for instance von Moltke L L, Greenblatt D J, Grassi J M, Granda B W, Venkatakrishnan K, Duan S X, Fogelman S M, Harmatz J S, Shader R I. (1999) Citalopram and desmethylcitalopram in vitro: human cytochromes mediating transformation, and cytochrome inhibitory effects. Biol Psychiatry 46(6): 839-49; Brosen K, Naranjo C A. (2001) Review of pharmacokinetic and pharmacodynamic interaction studies with citalopram. Eur Neuropsychopharmacol 11(4): 275-83.). It has also recently reported that metabolism of citalopram in blood occurs via monoamine oxidase B (MAO-B). As MAO is strongly expressed in human brain, this observation suggests that this enzymatic system may be implicated in drug metabolism in the CNS (see for instance Kosel M, Amey M, Aubert A C, Baumann P. (2001) In vitro metabolism of citalopram by monoamine oxidase B in human blood. Eur Neuropsychopharmacol 11(1): 75-8.).

Serotonin is transported from the synapse back into the pre-synaptic neuron to reduce synaptic levels. This action is mediated by the serotonin transporter protein (SERT). This transporter plays a pivotal role in the fine-tuning of serotonin neurotransmission (see for instance Blakely R D, De Felice L J, Hartzell H C. (1994) Molecular physiology of norepinephrine and serotonin transporters. J Exp Biol 196, 263-81.; Lesch K P, Meyer J, Glatz K, Flugge G, Hinney A, Hebebrand J, Klauck S M, Poustka A, Poustka F, Bengel D, Mossner R, Riederer P, Heils A. (1997) The 5-HT transporter gene-linked polymorphic region (5-HTTLPR) in evolutionary perspective: alternative biallelic variation in rhesus monkeys. Rapid communication. J Neural Transm 104, 1259-66.). SSRI's, including paroxetine and citalopram, preferentially bind to and inhibit the activity of the serotonin transporter (Weizman A, Weizman R. (2000) Serotonin transporter polymorphism and response to SSRIs in major depression and relevance to anxiety disorders and substance abuse. Pharmacogenomics 1(3): 335-41.; Goodnick P J, Goldstein B J. (1998) Selective serotonin reuptake inhibitors in affective disorders—I. Basic pharmacology. J Psychopharmacol 12(3 Suppl B): S5-20.). Citalopram has additionally been shown to reduce expression levels of this transporter (Horschitz S, Hummerich R, Schloss P. (2001) Structure, function and regulation of the 5-hydroxytryptamine (serotonin) transporter. Biochem Soc Trans 29(Pt 6): 728-32.). The serotonin transporter gene promoter region has an insertion/deletion polymorphism (5-HTTLPR; long 528 bp and short 484 bp), which is known to affect serotonin transporter expression and function (Lesch K P, Bengel D, Heils A, Sabol S Z, Greenberg B D, Petri S, Benjamin J, Muller C R, Hamer D H, Murphy D L. (1996) Association of anxiety-related traits with a polymorphism in the serotonin transporter gene regulatory region. Science 274, 1527-31.). The polymorphism is located approximately 1 kb upstream of the transcription initiation site consists of a 44-bp insertion or deletion and is composed of 16 repeat elements. Those with the short variant, approximately 42% of Caucasians, have reduced transcription of the 5-HTT gene promoter, resulting in decreased 5-HTT expression and an approximate 50% reduction in serotonin uptake (Heils A, Teufel A, Petri S, Stober G, Riederer P, Bengel D, Lesch K P. (1996) Allelic variation of human serotonin transporter gene expression. J Neurochem 66, 2621-4.; Collier D A, Stober G, Li T, Heils A, Catalano M, Di Bella D, Arranz M J, Murray R M, Vallada H P, Bengel D, Muller C R, Roberts G W, Smeraldi E, Kirov G, Sham P, Lesch K P. (1996) A novel functional polymorphism within the promoter of the serotonin transporter gene: possible role in susceptibility to affective disorders. Mol Psychiatry 1, 453-60.). Those with long/long genotype appear to respond more rapidly to paroxetine than those with one or two copies of the short allele (Kim D K, Lim S W, Lee S, Sohn S E, Kim S, Hahn C G, Carroll B J. (2000) Serotonin transporter gene polymorphism and antidepressant response. Neuroreport 11, 215-9.; Pollock B G, Ferrell R E, Mulsant B H, Mazumdar S, Miller M, Sweet R A, Davis S, Kirshner M A, Houck P R, Stack J A, Reynolds C F, Kupfer D J. (2000) Allelic variation in the serotonin transporter promoter affects onset of paroxetine treatment response in late-life depression. Neuropsychopharmacology 23, 587-90.). Citalopram has highly specific effects on the serotonin transport protein. Unlike some other SSRIs (e.g. paroxetine), it does not appreciably inhibit any other transporter.

Citalopram has primarily been reported to directly affect the serotonin receptors 5-HT1A/B and 5-HT2C. It is not reported to directly affect, in any significant manner, dopaminergic, adrenergic, histaminergic, sigma, or muscarinic receptors.

While citalopram does appear to affect the HPA axis via activation of glucocorticoid receptors, identification of components directly involved in this mechanism have not been identified.

In the present invention, these systems have been critically analyzed to select candidates for gene/SNP sets to be used as system inputs for our predictive algorithm. SNPs for selected genes are listed in FIG. 2, and we give as an example a summary of these systems for the SSRI citalopram.

Metabolism of Citalopram

The ability to assess differential metabolic rate of SSRIs is a basic requirement for the algorithm in our present invention. The rate of metabolism defines the half-life of a drug in the body and is a basic indicator of success or failure of the drug regimen. Failure of the drug regimen may occur either as non-responsiveness due to hypermetabolic activity, or as increased susceptibility to toxicity and interaction risk due to hypometabolic activity. Cytochrome P450 (CYP) isoenzymes play a major role in metabolism of citalopram.

Citalopram is N-demethylated to N-desmethylcitalopram partially by CYP2C19 and partially by CYP3A4. N-desmethylcitalopram is further N-demethylated by CYP2D6 to the likewise inactive metabolite di-desmethylcitalopram (von Moltke L L, Greenblatt D J, Grassi J M, Granda B W, Venkatakrishnan K, Duan S X, Fogelman S M, Harmatz J S, Shader R I. (1999) Citalopram and desmethylcitalopram in vitro: human cytochromes mediating transformation, and cytochrome inhibitory effects. Biol Psychiatry 46(6): 839-49., Brosen K, Naranjo C A. (2001) Review of pharmacokinetic and pharmacodynamic interaction studies with citalopram. Eur Neuropsychopharmacol 11(4): 275-83.). The two metabolites are not active. (Hiemke C, Hartter S. (2000) Pharmacokinetics of selective serotonin reuptake inhibitors. Pharmacol Ther 85(1): 11-28.). Because CYP2D6 is involved in metabolism of the metabolites of citalopram (which are not clinically active), generated by 3A4 and 2C19, we do not consider it a relevant selection for predicting response to citalopram treatment. We have selected CYP3A4 and 2C19; both of which are involved in metabolism of the clinically active citalopram.

It has also recently reported that metabolism of citalopram in blood occurs via monoamine oxidase B (MAO-B). As MAO is strongly expressed in human brain, this observation suggests that this enzymatic system may be implicated in drug metabolism in the CNS( Kosel M, Amey M, Aubert A C, Baumann P. (2001) In vitro metabolism of citalopram by monoamine oxidase B in human blood. Eur Neuropsychopharmacol 11(1): 75-8.). For this reason we have selected MAO-B.

Neurotransmitter Systems

This serotonin transporter protein (SERT) removes serotonin (5-HT) from the synapse. This transporter plays a pivotal role in the fine-tuning of serotonin neurotransmission. Citalopram is a selective serotonin reuptake inhibitor (SSRI) with a very high specificity for binding and inhibiting SERT (Weizman A, Weizman R. (2000) Serotonin transporter polymorphism and response to SSRIs in major depression and relevance to anxiety disorders and substance abuse. Pharmacogenomics 1(3): 335-41.). Citalopram has additionally been shown to reduce expression levels of this transporter (Horschitz S, Hummerich R, Schloss P. (2001) Structure, function and regulation of the 5-hydroxytryptamine (serotonin) transporter. Biochem Soc Trans 29(Pt 6): 728-32.). While other SSRIs have been shown to bind and inhibit the norepinephrine or dopamine transporters (e.g., paroxetine (Owens M J, Morgan W N, Ploft S J, Nemeroff C B. (1997) Neurotransmitter receptor and transporter binding profile of antidepressants and their metabolites. J Pharmacol Exp Ther 283(3): 1305-22., Owens M J, Knight D L, Nemeroff C B. (2000) Paroxetine binding to the rat norepinephrine transporter in vivo. Biol Psychiatry 47(9): 842-5.) and sertaline (Goodnick P J, Goldstein B J. (1998) Selective serotonin reuptake inhibitors in affective disorders—I. Basic pharmacology. J Psychopharmacol 12(3 Suppl B): S5-20.), respectively) citalopram has shown no such effects.

Citalopram has been demonstrated to have direct functional effects on the 5-HT1, 5-HT1B and 5-HT2C receptors. (Oerther S, Ahlenius S. (2001) Involvement of 5-HT1A and 5-HT1B receptors for citalopram-induced hypothermia in the rat. Psychopharmacology (Berl) 154(4): 429-34., Cremers T I, de Boer P, Liao Y, Bosker F J, den Boer J A, Westerink B H, Wikstrom H V. (2000) Augmentation with a 5-HT(1A), but not a 5-HT(1B) receptor antagonist critically depends on the dose of citalopram. Eur J Pharmacol 397(1): 63-74., Redrobe J P, MacSweeney C P, Bourin M. (1996) The role of 5-HT1A and 5-HT1B receptors in antidepressant drug actions in the mouse forced swimming test. Eur J Pharmacol 318(2-3): 213-20., Bolanos-Jimenez F, de Castro R M, Fillion G. (1993) Antagonism by citalopram and tianeptine of presynaptic 5-HT1B heteroreceptors inhibiting acetylcholine release. Eur J Pharmacol 242(1): 1-6., Ahlenius S, Larsson K. (1999) Synergistic actions of the 5-HT1A receptor antagonist WAY-100635 and citalopram on male rat ejaculatory behavior. Eur J Pharmacol 379(1): 1-6., Dekeyne A, Denorme B, Monneyron S, Millan M J. (2000) Citalopram reduces social interaction in rats by activation of serotonin (5-HT)(2C) receptors. Neuropharmacology 39(6): 1114-7., Millan M J, Girardon S, Dekeyne A. (1999) 5-HT2C receptors are involved in the discriminative stimulus effects of citalopram in rats. Psychopharmacology (Berl) 142(4): 432-4., Palvimaki E P, Roth B L, Majasuo H, Laakso A, Kuoppamaki M, Syvalahti E, Hietala J. (1996) Interactions of selective serotonin reuptake inhibitors with the serotonin 5-HT2c receptor. Psychopharmacology (Berl) 126(3): 234-40.) Although treatment with citalopram has been associated with alterations in the sensitivity of dopamine D3 receptors(Rogoz Z, Dziedzicka-Wasylewska M. (2000) Antidepressant drugs attenuate 7-OH-DPAT-induced hypoactivity in rats. Pol J Pharmacol 52(5): 331-6.), increase in D2 receptor expression (Kameda K, Kusumi I, Suzuki K, Miura J, Sasaki Y, Koyama T. (2000) Effects of citalopram on dopamine D2 receptor expression in the rat brain striatum. J Mol Neurosci 14(1-2): 77-86.) and alteration in the levels of mRNA for each subunit of the NMDA receptor(Boyer P A, Skolnick P, Fossom L H. (1998) Chronic administration of imipramine and citalopram alters the expression of NMDA receptor subunit mRNAs in mouse brain. A quantitative in situ hybridization study. J Mol Neurosci 10(3): 219-33.) there is no definitive evidence of its direct participation in affecting these or other (e.g., sigma1, sigma2, adrenergic, muscarinic, histaminergic) neurotransmitter systems. Receptor genes selected in this case are 5-HT1A, 5-HT1B and 5-HT2C.

Hypothalamic-Pituitary-Adrenal (HPA) Axis

Glucocorticoid receptor (GR) activation by glucocorticoids, with subsequent binding to and activation of the glucocorticoid responsive element, has been shown to be necessary component of the cortisol feedback loop of the hypothalamus-pituitary-adrenal (HPA) axis(Spencer R L, Kim P J, Kalman B A, Cole M A. (1998) Evidence for mineralocorticoid receptor facilitation of glucocorticoid receptor-dependent regulation of hypothalamic-pituitary-adrenal axis activity. Endocrinology 139(6): 2718-26.). Abnormalities that result in attenuation of GR functionality and/or levels have been proposed to underlie hyperactivity of the HPA axis as described in patients with major depression(Pariante C M, Miller A H. (2001) Glucocorticoid receptors in major depression: relevance to pathophysiology and treatment. Biol Psychiatry 49(5): 391-404.). Perhaps the most striking support of the hypothesis that abnormalities in the GR contribute to the pathophysiology of major depression derives from studies suggesting that antidepressants may exert their clinical effects through direct modulation of the GR(Pariante C M, Miller A H. (2001) Glucocorticoid receptors in major depression: relevance to pathophysiology and treatment. Biol Psychiatry 49(5): 391-404.). Additional support of this hypothesis is the observation that transgenic mice with disturbed GR function display several characteristics seen in depressive illness, including a hyperactive HPA axis(Barden N, Stec I S, Montkowski A, Holsboer F, Reul J M. (1997) Endocrine profile and neuroendocrine challenge tests in transgenic mice expressing antisense RNA against the glucocorticoid receptor. Neuroendocrinology 66(3): 212-20.).

Citalopram has been shown to induce GR translocation from the cytoplasm to the nucleus, thereby enhancing GR-mediated gene transcription(Pariante C M, Makoff A, Lovestone S, Feroli S, Heyden A, Miller A H, Kerwin R W. (2001) Antidepressants enhance glucocorticoid receptor function in vitro by modulating the membrane steroid transporters. Br J Pharmacol 134(6): 1335-43.). Support of citalopram's role in resolution of dysfunctional HPA axis signaling, via the aforementioned action, is derived from the observation that while in depressed patients cortisol responses are blunted, they are not in subjects recovered using citalopram (Bhagwagar Z, Whale R, Cowen P J. (2002) State and trait abnormalities in serotonin function in major depression. Br J Psychiatry 180(1): 24-28.).

### EXAMPLES

### Example 1

### Citalopram

A four-week study of 118 severely depressed patients on the molecule citalopram was performed. Severely Depressed patients were defined to have a HAM-D score of 18 or greater. The patients were newly diagnosed and not on any previous psychotropic medication. The molecule citalopram was used in this study.

As an independent measure, a ≧50% decrease in HAM-D score may be considered a response (Lecrubier Y, Clerc G, Didi R, Kieser M. Related Articles, Links Abstract Efficacy of St. John's wort extract WS 5570 in major depression: a double-blind, placebo-controlled trial. Am J Psychiatry. August 2002;159(8):1361-6.). However, we decided that using three different measures of emotional state that were averaged would provide a more reliable assessment of response than the HAM-D test alone.

Therefore, scores from the HAM-D, the Emotional State (Depression Section Only; Dep), and the final (4 week) CGI-I score were used to determine patient response to the treatment with Citalopram.

While the Dep section of the Emotional State questionnaire and HAM-D are scored both before and after treatment to obtain change in score, the final CGI-I is a single score (from 1 to 7) given by the doctor at the end of the study assessing the patient's improvement. In order to take an average of all three scores it was necessary to scale the CGI-I test score to that of both the HAM-D and Dep scores.

For both HAM-D and Dep tests a 50% decrease in score (response quotient of 0.50) was considered a minimal response. With this as the point of reference, we assigned the CGI-I ‘Minimally improved’ score of 3, a response quotient of 0.50. ‘Very much improved’ was assigned a response quotient of 1.00. ‘Minimally worse’ was assigned a response quotient of 0.00. All patients in this study scored between 1 and 5 on the CGI-I.

Outcome Measure

As the study inclusion criteria was a minimum HAM-D score at week 0, the validity of this week 0 HAM-D for assessing outcome as a ratio of HAM-D from week 4 to week 0 was questionable. Therefore, the relationship between HAM-D and CGI-S was assessed over course of study, week by week, and the relationship at week 0 was contrasted with weeks 2 and 4. The correlation between CGI-S and HAM-D for weeks 2 and 4 were determined with constrained linear regression, having an offset of zero. The slopes of the fits between weekly HAM-D and CGI-S and their corresponding 95% confidence intervals for weeks 0, 2 and 4, were 4.9 (4.8 5.0), 4.0 (4.1-4.4), and 4.4 (3.8-4.1), respectively. See FIG. 4. Jointly for weeks 2 and 4, the slope of the correlation was be 4.1 (4.0-4.2) consistent for week 2 and week 4, but inconsistent with week 0 (p<0.05). See FIG. 5.

HAM-D week 0 was biased towards individuals initially self-reporting as more depressed than expected given the corresponding CGI-S inventories. Consequently, HAM-D baseline week 0 scores were confounded for assessing response to the therapy, and were not be used in scoring outcome.

To overcome the confound in the intake score, the values for HAM-D week 0 discarded for use in the outcome measure, and a baseline HAM-D was imputed from CGI-S week 0 and the relationship between CGI-S and HAM-D in weeks 2 and 4 was determined as follows:
HAM-D≈0.0+CGI-S*4.1 (1)

The imputed HAM-D (week 0) was then used as a normalization factor in an outcome-measure comprising week 4 HAM-D and scaled CGI-S week 4. The outcome measure represents the ratio of the subjects' depression inventory at week 4 to week 0:
Y₀=0.0+CGI-S*4.1 (2)
Y₄=(CGI-S(wk4)*4.1+HAM-D(wk4))/2 (3)
Y=Y₄/Y₀ (4)

To account for ambiguity in assessment, a response criteria of <0.5 was imposed. The subjects were then grouped by their outcome measure score Y into responders and non-responders.

Demographics of Patient Data

Following completion of the 4 week study we had collected 118 patient samples with: (1) response data, and (2) genotype information for 91 SNPs. Of the total 118 samples there were 68 Responders and 50 Non-Responders. The median age of the subjects was 35 years, with 5% and 95% intervals corresponding to 19 and 61 years. The majority of the patients experienced recurring depression (n=87). All of the patients completed the study.

Clinical Methods

The subjects were treated with Citalopram for 4 weeks with a self-administered 20 mg daily dose. The subjects were seen in follow-up at 2 and 4 weeks and assessed with HAM-D and Clinical Global Impression—Severity (CGI-S) inventories at weeks 0, 2 and 4. Background Clinical information was also obtained. After the study was completed, subjects were profiled for 96 Single Nucleotide Polymorphisms (SNPs) in genes related to the action of Citalopram. The selected SNPs are listed in FIG. 1.

As a first step, a linear association analysis was performed to screen for “Golden SNPs”, single SNPs that could be used independently to predict response. Since depression is a complex disease involving many genes as detailed above, we did not expect to find any, however, these SNPs alone or in combination could be relevant to disease prediction in smaller subgroups of people.

We found no “Golden SNP” that delivered predictive success greater than 62%. Using a simple binary predictor, which counts the number of each outcome category for each genotype and assigns an outcome for that genotype based on the outcome category with the highest count, we identified the top performing individual SNP to have a Predictive Success (i.e., % Correct) of 62.4%. This SNP is located in the monoamine oxidase A (MAOA) gene. FIG. 2 lists the results for the top 12 performing SNPs in this analysis.

Predictive Success is defined to be (percentage correctly predicted)×(1−percentage laundered). Laundering is a dynamic process that evaluates whether a SNP genotype combination is found in both the responder and non-responder patient groups. Those patient samples that have SNP genotype combinations that occur for both responders and non-responders are removed from the dataset before the neural net is trained, tested and evaluated. When looking at a 2 SNP input combination the degree of laundering is high (perhaps >65% of samples are removed). However, as the SNP genotype input number increases, the likelihood of finding the same genotype combination in both the responder and non-responder groups becomes low and, hence, the degree of laundering decreases (perhaps <10% of samples are removed).

At this point it was postulated, that while independently the best SNP genotype perform poorly, by grouping them together as inputs to a nonlinear classifier, an increased Predictive Success might be achieved. This would say that the genetics of depression SSRI medication response are highly linear.

It is worth noting here, for clarification, that the inputs to the classifiers discussed here are not the patient's genotypes themselves (e.g., AT, or a numerical representation of the input), but whether or not a patient has a specific SNP genotype (e.g., 0 or 1). For instance, if one input in a classifier is classified as ‘SNP#1234 Genotype AT’, if a patient has the AT genotype at this SNP position, the input value will be ‘1’. If a patient has genotype AA at this position instead, the input value will be ‘0’.

We tested this hypothesis using stepped combinations of the SNP genotypes listed in Table 1 as inputs to develop a neural net (i.e., the first neural net used the top 2 SNP genotypes as inputs, the next used the top 3 SNP genotypes as inputs, the next used the top 4 SNPs, etc.). While two combinations performed slightly better (up to 65%), most of the combinations fared worse than the best SNP being used as an independent predictor. This is shown in FIG. 3.

Our conclusions from this analysis are: (1) of the SNPs we have selected for analysis, there is no golden SNP, and (2) the highest performing linear correlates of this dataset do not appear to be complimentary in predicting response in building a predictor.

It appears, based on our available dataset, that individual patient response to depression medication, particularly citalopram, is a complex process involving multiple components. These components do not appear to have significant first order linear associations, and may instead be encompassed by non-linear interactions (second order and above) between SNPs. To develop a successful predictor it will be necessary to identify a SNP combination that detects and exploits interactions between SNPs to differentiate responders from non-responders. This was done and the markers found in part comprise the present invention.

We have established that linear techniques do not provide a combination of biomarkers with an acceptable level of accuracy for prediction of response/nonresponse (R/NR) of citalopram to the clinician. It will be necessary to identify a sub-set of SNPs or SNP genotypes with non-linear associations that contributes to predicting outcome. A global search algorithm was used, described in patent application Ser. No. 09/611,220 and subsequent divisional applications and incorporated within by reference, to winnow down the number of possible combinations of SNPs from 91!˜10¹⁵⁷ to those that are the most predictive of response or nonresponse to citalopram in a patient population.

Modeling Methods

SNP Coding

The SNP data was transformed into the equivilent alleles feature set from the alleles observed for each SNP. This was achieved by coding each unique allele of each SNP as an integer, and then forming a binary representation of the alleles observed for each SNP. This resulted in a feature set dimension of 329.

The goal was to select a set of relevant features from the complete set of SNPs that resulted in a predictive relationship for response or non-response to Citalopram. Feature selection and model parameterization were performed jointly with 5 fold cross-validated Naïve testing and 5 fold cross validation on the training utilizing custom algorithms implemented in Matlab 6.5. This procedure consists of forming 5 nearly disjoint testing sets and then for each of these testing sets forming 5 training/cross-validation sets. Data stratification is maintained with respect to the outcome measure while forming these sets so there is roughly equal representation of the original data set outcome distribution in each of the individual training, cross-validation, and Naïve testing sets.

Feature Selection

Features were selected using a forward/backward search strategy to build a CART based model. At each step of the forward recursion, a search was performed to maximize negative predictive value as a primary goal or positive predictive value as a secondary goal. This approach was selected as it was hypothesized that polymorphisms in the relevant proteins would be more likely to interfere with the action of the therapeutic compound rather than enhancing the action of the therapeutic compound on alleviating depression. The search criteria was expressed with as a negative predictive ratio for the new feature being added to be greater than a selection threshold, constrained by a maximum false negative prediction rate for the cross-validation training set. The search algorithm was also constrained by a minimum number of positive and negative predictions, respectively, to minimize the impact of spurious feature selections due to small sample size. If no features satisfied the initial criteria of a negative predictive ratio of 15, then the threshold was decreased by increments of 0.5 to 2 and the search resumed. However, if a negative predictive ratio was not identified, then the ranges on the constraints of the minimum sample size of positive and negative predictions were decreased in increments of 1 from 8 to 4. If a feature could not be added to satisfy the primary goal, the secondary goal was evaluated in the same manner, but with the search criteria applied for positive predictive ratio and false positive predictions.

The goal of maximizing negative predictive value was expressed with joint selection criteria of negative predictive ratios greater then selection thresholds and feature(s) corresponding to a minimum false negative prediction rate for the cross-validation training set. The search algorithm was also constrained by a minimum number of positive and negative predictions, respectively, to minimize the impact of spurious feature selections due to small sample size. If no features satisfied the goals of negative predictive ratios given the initial threshold of 15, then the threshold was decreased by increments of 0.5 and the search resumed. However, if a negative predictive ratio was not identified, then the ranges on the constraints of the number of positive and negative predictions were decreased in increments of 1 from 8 to 4.

After each forward selection phase, feature removal was employed in order to select the model structure that best identified responders and non-responders. This was accomplished by identifying the feature set that maximized prediction accuracy.

After forward feature selection, pruning was employed by calculating the performance of the cross validation set while recursively removing terminal nodes. Pruning was performed in order to select the model structure that best identified responders and non-responders by maximizing predictive value at each of the four prediction bins.

Post Processing

Finally, after model completion, the degree of representation for each of the bins from each of the models was assessed, and bins 1 and 2 were combined as only one of the five models had bin 1 predictions.

The finalized models were then applied to their corresponding Naïve test sets to check for statistical consistency in feature selection and model parameterization.

The SNPs selected based on this joint selection and model parameterization method for the five data-model sets and the corresponding frequencies of the SNPS in the model set is given in FIG. 6.

The average cross-validation and the Naïve testing performance of the model set spanning the complete set of subjects is given in FIG. 7.

In total, 20 SNPs were included in the five models constructed. Four SNPs were included in all of the models, which are in genes coding for the Dopamine Receptor D2 and the Solute Carrier Family 6 (neurotransmitter transporter, serotonin), member 4, and 2 unknowns. Five SNPs were included in two or more models: CRHR1 corticotropin releasing hormone receptor 1, DRD2 dopamine receptor D2, TXNRD2 thioredoxin reductase 2, COMT catechol-O-methyltransferase, and CYP3A4 cytochrome P450, family 3, subfamily A, polypeptide 4. Of the remaining 12 SNPS selected, 10 code for one of two classes of proteins, either a 5-hydroxytryptamine (serotonin) receptor SNP (5 instances), or an ATP-binding cassette, sub-family B (MDR/TAP) (5 instances).

Of the commonly identified SNPS in the five models, the probability of response for subjects with either rs1076560 allele TC (DRD2 dopamine receptor D2) and/or rs1972305 not allele TC (SLC6A4 solute carrier family 6 [neurotransmitter transporter, serotonin], member 4) decreases the probability of response to less than 20% (n=37) (p<0.01) and occurs with a false negative rate of 12%. Of the 5 subjects whom were identified as responding in spite of the presence of these alleles, their outcome scores were marginally below 0.5 mean 46(0.04).

The presence of rs2174444 allele TT decreases the probability of response to less than 30% (n=21) (p<0.05), and occurs with a false negative rate of 14%. Of the 6 subjects whom responded in spite of these alleles, their outcome scores were marginally below 0.5, mean 0.47(0.03).

The presence of any of these three alleles, decreases the probability of response to less than 25% but occurs with a false negative rate of 40%. Hence there is a need for a more advanced means of combining the features to produce a model with lower false negative rates and greater applicability.

The models produced from the forward feature selection to maximize negative predictive power and minimize false negative rate were able to successfully identify patients with increased and decreased probabilities of response compared to the average population response rates in the study. The model produces three bins (1-3) as shown in FIG. 7. Bin 1 corresponds to responding subjects and has a false positive rate on naïve data of <20%. Bin 3 corresponds to non-responders and has a false negative rate of 24%. Bin 2 corresponds to subjects that had a probability of response of less than 60%, but could not be predicted to be clear non-responders. This bin corresponded to only 14% of the subjects in the study. This bin 2 adds flexibility to the interpretation of the model for clinical implementation, by identifying likely but not definite non-responders.

We have examined and ruled out the possibility that random chance is responsible for the strong positive results we are achieving by testing the global search algorithm against a random SNP dataset. It would not be unreasonable to question whether a 118 patient sample group could be partitioned into responders and non-responders using 96 random variables. Upon examination of this possibility by subjecting a random dataset identical in dimension to that of the citalopram dataset (e.g. 91 SNPs, 118 patients) to the forward search algorithm described above, we found our technology was unable to select any combinations of random variables with a predictive ability greater than 55%. This supports our conclusion that we have identified select SNPs with relevant information for predicting outcome and that nonlinear algorithms are capable of extracting minimally representative information contained in complex multi-variable groups.

In a preferred embodiment of the present invention, to enable higher predictive accuracy, one can use the top N SNP groups to train a committee network, described below, in a voting scheme. Basically N predictors of N sets of groups each give a “vote” to new, previously unseen examples presented to each predictor. The votes are added up and a final output is given based upon this “group vote”. This methodology with the dataset yielded a predictive accuracy of 89±2%.

In still another preferred embodiment of the present invention, one or more of the top 50 SNP groups, given below, found might work better singly or in combination with other SNP groups with a certain subsection of the population. One can then train a predictor algorithm with these specific combinations.

Said specific combinations are the following, put into vertical columns labeled one through fifty:

| 1 | 2 | 3 | 4 | 5 |
|---|---|---|---|---|
| MAOA 979606 | MAOA 979606 | CRHR1 242924 | CRHR1 242924 | MAOA 979606 |
| SLC6A4 1972305 | SLC6A4 1972305 | CRHR2 929377 | CRHR2 929377 | SLC6A4 1972305 |
| ABCB1 1202169 | ABCB1 1202169 | MAOA 979606 | CYP3A4 2246709 | ABCB1 1202169 |
| ABCB1 1055302 | ABCB1 1055302 | HTR1B 6296 | HTR3A 1062613 | ABCB1 1055302 |
| CRHBP 964734 | CRHBP 964734 | MAOB 1181252 | SLC6A3 1042098 | CRHBP 964734 |
| COMT 165688 | COMT 165688 | SLC6A4 1972305 | CRHBP 2174444 | COMT 165688 |
| MAOB 2311013 | MAOB 2311013 | ABCB1 1202186 | CYP3A4 1851426 | MAOB 2311013 |
| | MAOB 2056913 | DRD2 6278 | | MAOB 2056913 |
| | HTR3A 1062613 | ABCB1 1202169 | | |
| 6 | 7 | 8 | 9 | 10 |
| CRHR1 242924 | CRHR1 242924 | MAOA 979606 | MAOA 979606 | MAOA 979606 |
| CRHR2 929377 | CRHR2 929377 | SLC6A4 1972305 | SLC6A4 1972305 | SLC6A4 1972305 |
| CYP3A4 2246709 | MAOA 979606 | ABCB1 1202169 | ABCB1 1202169 | ABCB1 1202169 |
| HTR3A 1062613 | HTR1B 6296 | ABCB1 1055302 | ABCB1 1055302 | ABCB1 1055302 |
| SLC6A3 1042098 | MAOB 1181252 | CRHBP 964734 | CRHBP 964734 | CRHBP 964734 |
| CRHBP 2174444 | SLC6A4 1972305 | MAOB 736944 | MAOB 736944 | MAOB 736944 |
| | ABCB1 1202186 | HTR2A 6313 | HTR2A 6313 | HTR2A 6313 |
| | DRD2 6278 | MAOB 2311013 | MAOB 2311013 | MAOB 2311013 |
| | MAOB 1799836 | | CYP3A4 1851426 | |
| 11 | 12 | 13 | 14 | 15 |
| CRHR1 242924 | MAOA 979606 | CRHR1 242924 | CRHR1 242924 | MAOA 979606 |
| CRHR2 929377 | SLC6A4 1972305 | CRHR2 929377 | CRHR2 929377 | SLC6A4 1972305 |
| MAOA 979606 | ABCB1 1202169 | MAOA 979606 | MAOA 979606 | ABCB1 1202169 |
| HTR1B 6296 | ABCB1 1055302 | HTR1B 6296 | HTR1B 6296 | ABCB1 1055302 |
| MAOB 1181252 | CRHBP 964734 | MAOB 1181252 | MAOB 1181252 | CRHBP 964734 |
| SLC6A4 1972305 | MAOB 736944 | SLC6A4 1972305 | SLC6A4 1972305 | MAOB 736944 |
| ABCB1 1202186 | HTR2A 6313 | MAOB 1799836 | ABCB1 1202186 | HTR2A 6313 |
| SLC6A3 37022 | MAOB 2311013 | CYP3A4 2246709 | DRD2 6278 | MAOB 2311013 |
| | COMT 165688 | | | COMT 165688 |
| | CYP3A4 2246709 | | | |
| 16 | 17 | 18 | 19 | 20 |
| CRHR1 242924 | CRHR1 242924 | MAOA 979606 | MAOA 979606 | CRHR1 242924 |
| CRHR2 929377 | CRHR2 929377 | SLC6A4 1972305 | SLC6A4 1972305 | CRHR2 929377 |
| MAOA 6323 | MAOA 979606 | ABCB1 1202169 | ABCB1 1202169 | MAOA 979606 |
| ABCB1 1858923 | HTR1B 6296 | ABCB1 1055302 | ABCB1 1055302 | HTR1B 6296 |
| CYP3A4 2246709 | MAOB 1181252 | CRHBP 964734 | CRHBP 964734 | MAOB 1181252 |
| MAOA 6355 | SLC6A4 1972305 | MAOB 736944 | MAOB 736944 | SLC6A4 1972305 |
| HTR2B 1549339 | ABCB1 1202186 | HTR2A 6313 | HTR2A 6313 | MAOA 6323 |
| MAOB 2311013 | MAOB 2311013 | MAOB 2311013 | MAOB 2311013 | CYP3A4 2246709 |
| | | COMT 165688 | COMT 165688 | |
| | | HTR2A 3125 | HTR2A 3125 | |
| | | MAOB 1181252 | MAOB 1181252 | |
| | | HTR2A 6312 | HTR2A 6312 | |
| | | MAOA 6355 | MAOA 6355 | |
| | | | MAOB 2311013 | |
| 21 | 22 | 23 | 24 | 25 |
| CRHR1 242924 | CRHR1 242924 | MAOA 979606 | CRHR1 242924 | CRHR1 242924 |
| CRHR2 929377 | CRHR2 929377 | SLC6A4 1972305 | CRHR2 929377 | CRHR2 929377 |
| MAOA 979606 | MAOA 979606 | ABCB1 1202169 | MAOA 6323 | MAOA 979606 |
| HTR1B 6296 | HTR1B 6296 | ABCB1 1055302 | ABCB1 1858923 | HTR1B 6296 |
| MAOB 1181252 | MAOB 1181252 | CRHBP 964734 | CYP3A4 2246709 | MAOB 1181252 |
| SLC6A4 1972305 | SLC6A4 1972305 | MAOB 736944 | DRD2 6278 | SLC6A4 1972305 |
| MAOA 6355 | ABCB1 1202186 | HTR2A 6313 | | MAOA 6355 |
| CYP3A4 2246709 | | MAOB 2311013 | | ABCB1 1202169 |
| | | COMT 165688 | | |
| | | HTR2A 3125 | | |
| | | MAOB 1181252 | | |
| | | HTR2A 6312 | | |
| | | MAOA 6355 | | |
| | | SLC6A3 403636 | | |
| 26 | 27 | 28 | 29 | 30 |
| MAOA 979606 | CRHR1 242924 | MAOA 979606 | CRHR1 242924 | CRHR1 242924 |
| SLC6A4 1972305 | CRHR2 929377 | SLC6A4 1972305 | CRHR2 929377 | CRHR2 929377 |
| ABCB1 1202169 | MAOA 6323 | ABCB1 1202169 | CYP3A4 2246709 | MAOA 6323 |
| ABCB1 1055302 | ABCB1 1858923 | ABCB1 1055302 | HTR3A 1062613 | ABCB1 1858923 |
| CRHBP 964734 | CYP3A4 2246709 | CRHBP 964734 | SLC6A3 1042098 | CYP3A4 2246709 |
| MAOB 736944 | DRD2 1125394 | MAOB 736944 | CRHBP 2174444 | MAOA 6355 |
| HTR2A 6313 | HTR2B 1549339 | HTR2A 6313 | HTR2B 1549339 | HTR2B 1549339 |
| MAOB 2311013 | | MAOB 2311013 | | |
| COMT 165688 | | COMT 165688 | | |
| DRD2 6276 | | HTR2A 3125 | | |
| HTR2A 3125 | | MAOB 1181252 | | |
| | | HTR2A 6312 | | |
| | | MAOA 6355 | | |
| | | SLC6A3 403636 | | |
| 31 | 32 | 33 | 34 | 35 |
| MAOA 979606 | MAOA 979606 | CRHR1 242924 | MAOA 979606 | CRHR1 242924 |
| SLC6A4 1972305 | SLC6A4 1972305 | CRHR2 929377 | SLC6A4 1972305 | CRHR2 929377 |
| ABCB1 1202169 | ABCB1 1202169 | MAOA 979606 | ABCB1 1202169 | CYP3A4 2246709 |
| ABCB1 1055302 | ABCB1 1055302 | HTR1B 6296 | ABCB1 1055302 | HTR3A 1062613 |
| CRHBP 964734 | CRHBP 964734 | MAOB 1181252 | CRHBP 964734 | SLC6A3 1042098 |
| MAOB 736944 | MAOB 736944 | SLC6A4 1972305 | MAOB 736944 | CRHBP 2174444 |
| HTR2A 6313 | HTR2A 6313 | MAOA 6323 | | CYP3A4 1851426 |
| MAOB 2311013 | MAOB 2311013 | CYP3A4 2246709 | | HTR3A 1150226 |
| COMT 165688 | COMT 165688 | HTR2A 594242 | | |
| HTR2A 3125 | HTR1A 1800044 | | | |
| MAOB 1181252 | | | | |
| HTR2A 6312 | | | | |
| MAOA 6355 | | | | |
| SLC6A3 403636 | | | | |
| SLC6A3 1042098 | | | | |
| 36 | 37 | 38 | 39 | 40 |
| CRHR1 242924 | CRHR1 242924 | CRHR1 242924 | CRHR1 242924 | CRHR1 242924 |
| CRHR2 929377 | CRHR2 929377 | CRHR2 929377 | CRHR2 929377 | CRHR2 929377 |
| CYP3A4 2246709 | MAOA 979606 | CYP3A4 2246709 | MAOA 6323 | MAOA 979606 |
| HTR3A 1062613 | HTR1B 6296 | HTR3A 1062613 | ABCB1 1858923 | HTR1B 6296 |
| HTR2A 3125 | MAOB 1181252 | SLC6A3 1042098 | CYP3A4 2246709 | MAOB 1181252 |
| ABCB4 1202283 | SLC6A4 1972305 | CRHBP 2174444 | DRD2 1125394 | SLC6A4 1972305 |
| | MAOB 1181252 | ABCB1 1202179 | | MAOA 6355 |
| 41 | 42 | 43 | 44 | 45 |
| CRHR1 242924 | MAOA 979606 | CRHR1 242924 | CRHR1 242924 | CRHR1 242924 |
| CRHR2 929377 | SLC6A4 1972305 | CRHR2 929377 | CRHR2 929377 | CRHR2 929377 |
| MAOA 6323 | ABCB1 1202169 | MAOA 6323 | MAOA 6323 | MAOA 979606 |
| ABCB1 1858923 | ABCB1 1055302 | ABCB1 1858923 | ABCB1 1858923 | HTR1B 6296 |
| CYP3A4 2246709 | CRHBP 964734 | CYP3A4 2246709 | CYP3A4 2246709 | MAOB 1181252 |
| MAOB 2311013 | MAOB 736944 | DRD2 1124491 | HTR2A 6311 | SLC6A4 1972305 |
| | HTR2A 6313 | | | ABCB1 1202186 |
| | MAOB 2311013 | | | HTR1B 6298 |
| | COMT 165688 | | | CYP3A4 2246709 |
| | HTR1A 1800044 | | | |
| | COMT 4633 | | | |
| 46 | 47 | 48 | 49 | 50 |
| CRHR1 242924 | CRHR1 242924 | CRHR1 242924 | MAOA 979606 | MAOA 979606 |
| CRHR2 929377 | CRHR2 929377 | CRHR2 929377 | SLC6A4 1972305 | SLC6A4 1972305 |
| MAOA 979606 | MAOA 979606 | MAOA 6323 | ABCB1 1202169 | ABCB1 1202169 |
| HTR1B 6296 | HTR1B 6296 | ABCB1 1858923 | ABCB1 3842 | ABCB1 1055302 |
| MAOB 1181252 | MAOB 1181252 | CYP3A4 2246709 | CRHBP 964734 | CRHBP 964734 |
| SLC6A4 1972305 | SLC6A4 1972305 | CRHR2 2014663 | MAOA 2205718 | MAOB 736944 |
| ABCB1 1202186 | | | SLC6A3 365663 | HTR2A 6313 |
| DRD2 6278 | | | MAOB 1181252 | MAOB 2311013 |
| MAOB 1799836 | | | | COMT 165688 |
| CRHBP 2174444 | | | | HTR2A 3125 |

### Example II

### Paroxetine

Pharmacogenomics of Paroxetine in Treating Depression

In recent years, the search for a single gene responsible for major depressive disorder has given way to the understanding that multiple gene variants, acting together with yet unknown environmental risk factors or developmental events, interact in a complex system to account for its expression phenotype. In accordance, treatments that successfully alleviate depression symptoms are likely to act on multiple gene products.

A popular hypothesis of the pathophysiology of depression, called the monoamine hypothesis, proposes that the underlying pathophysiologic basis of depression is a depletion in the levels of serotonin, norepinephrine, and/or dopamine in the central nervous system. This is supported by the mechanism of action of antidepressants, which is to elevate the levels of these neurotransmitters in the brain (R. Tissot. The common pathophysiology of monaminergic psychoses: a new hypothesis. Neuropsychobiology 1, 243-60 (1975)).

Paroxetine has proven to be an effective treatment in this regard Although classified as an SSRI, with preferential inhibition of the serotonin transport protein (resulting in increases in synaptic levels of serotonin with resultant serotonin autoreceptor desensitization), paroxetine also exerts significant inhibitory effects on the norepinephrine transport protein (NET) (M. J. Owens, W. N. Morgan, S. J. Plott, C. B. Nemeroff. Neurotransmitter receptor and transporter binding profile of antidepressants and their metabolites. J Pharmacol Exp Ther 283, 1305-22 December 1997; M. J. Owens, D. L. Knight, C. B. Nemeroff. Paroxetine binding to the rat norepinephrine transporter in vivo. Biol Psychiatry 47, 842-5 May 1, 2000). Additionally, it has been reported that dopamine receptor sensitivity is a predictive factor in the responsiveness of treatment to paroxetine (E. Healy, P. McKeon. Dopaminergic sensitivity and prediction of antidepressant response. J Psychopharmacol 14, 152-6 (June 2000). These systems, and others proposed to be involved in depression and affected by paroxetine (e.g., HPA axis (W. Pitchot, C. Herrera, M. Ansseau. HPA axis dysfunction in major depression: relationship to 5-HT(1A) receptor activity. Neuropsychobiology 44, 74-7 (2001))), have been critically analyzed to identify candidates for gene/SNP sets to be used as system inputs for a predictive algorithm to predict response to anti-depressant treatment.

Metabolism of Paroxetine: P450 (CYP2D6)

The rate of metabolism defines the half-life of paroxetine in the body and is a basic indicator of success or failure of the drug regimen. Failure can occur either as non-responsiveness due to hypermetabolic activity, or as increased susceptibility to toxicity and interaction risk due to hypometabolic activity.

Paroxetine is both metabolized by and inhibits the CYP2D6 gene product (Bourin M, Chue P, Guillon Y. (2001) Paroxetine: a review. CNS Drug Rev 7, 25-47) which is part of the cytochrome P450 group (Ingelman-Sundberg M, Evans W E. (2001) Unravelling the functional genomics of the human CYP2D6 gene locus. Pharmacogenetics 11, 553-4). This is the only protein reported to be involved in metabolism of paroxetine. Polymorphisms in CYP2D6 that modulate the enzyme's ability to metabolize paroxetine versus wild-type (Ramamoorthy Y, Tyndale R F, Sellers E M. (2001) Cytochrome P450 2D6.1 and cytochrome P450 2D6.10 differ in catalytic activity for multiple substrates. Pharmacogenetics 11, 477-87), have been defined.

Serotonergic System

Serotonin has the molecular structure of 5-hydroxytryptamine, 5-HT, a molecule derived from the amino acid tryptophan. The rate-limiting enzyme in the biosynthesis of serotonin is tryptophan hydroxylase (TPH). Abnormalities in TPH activity have been implicated in a wide range of psychiatric disorders (Abbar M, Courtet P, Amadeo S, Caer Y, Mallet J, Baldy-Moulinier M, Castelnau D, Malafosse A. (1995) Suicidal behaviors and the tryptophan hydroxylase gene. Arch Gen Psychiatry 52, 846-9). The A218C polymorphism in tryptophan hydroxylase has been associated with the antidepressant activity of paroxetine. It has been demonstrated that TPH*A/A and TPH*A/C variants were associated with a poorer response to paroxetine treatment when compared to TPH*C/C (P=0.005). TPH gene variants are therefore a possible modulator of paroxetine antidepressant activity (Serrefti A, Zanardi R, Cusin C, Rossini D, Lorenzi C, Smeraldi E. (2001) Tryptophan hydroxylase gene associated with paroxetine antidepressant activity. Eur Neuropsychopharmacol 11, 375-80).

Serotonin is transported from the synapse back into the pre-synaptic neuron to reduce synaptic levels. This action is mediated by the serotonin transporter protein (SERT). This transporter plays a pivotal role in the fine-tuning of serotonin neurotransmission (Blakely R D, De Felice L J, Hartzell H C. (1994) Molecular physiology of norepinephrine and serotonin transporters. J Exp Biol 196, 263-81.; Lesch K P, Meyer J, Glatz K, Flugge G, Hinney A, Hebebrand J, Klauck S M, Poustka A, Poustka F, Bengel D, Mossner R, Riederer P, Heils A. (1997) The 5-HT transporter gene-linked polymorphic region (5-HTTLPR) in evolutionary perspective: alternative biallelic variation in rhesus monkeys. Rapid communication. J Neural Transm 104, 1259-66.). SSRI's, including paroxetine, preferentially bind to and inhibit the activity of the serotonin transporter (Weizman A, Weizman R. (2000) Serotonin transporter polymorphism and response to SSRIs in major depression and relevance to anxiety disorders and substance abuse. Pharmacogenomics 1, 335-41). The serotonin transporter gene promoter region has an insertion/deletion polymorphism (5-HTTLPR; long 528 bp and short 484 bp), which is known to affect serotinin transporter expression and function (Lesch K P, Bengel D, Heils A, Sabol S Z, Greenberg B D, Petri S, Benjamin J, Muller C R, Hamer D H, Murphy D L. (1996) Association of anxiety-related traits with a polymorphism in the serotonin transporter gene regulatory region. Science 274, 1527-31). The polymorphism is located approximately 1 kb upstream of the transcription initiation site consists of a 44-bp insertion or deletion and is composed of 16 repeat elements. Those with the short variant, approximately 42% of Caucasians, have reduced transcription of the 5-HTT gene promoter, resulting in decreased 5-HTT expression and an approximate 50% reduction in serotonin uptake (Heils A, Teufel A, Petri S, Stober G, Riederer P, Bengel D, Lesch K P. (1996) Allelic variation of human serotonin transporter gene expression. J Neurochem 66, 2621-4; Collier D A, Stober G, Li T, Heils A, Catalano M, Di Bella D, Arranz M J, Murray R M, Vallada H P, Bengel D, Muller C R, Roberts G W, Smeraldi E, Kirov G, Sham P, Lesch K P. (1996) A novel functional polymorphism within the promoter of the serotonin transporter gene: possible role in susceptibility to affective disorders. Mol Psychiatry 1, 453-60). Those with long/long genotype appear to respond more rapidly to paroxetine than those with one or two copies of the short allele (Kim D K, Lim S W, Lee S, Sohn S E, Kim S, Hahn C G, Carroll B J. (2000) Serotonin transporter gene polymorphism and antidepressant response. Neuroreport 11, 215-9; Pollock B G, Ferrell R E, Mulsant B H, Mazumdar S, Miller M, Sweet R A, Davis S, Kirshner M A, Houck P R, Stack J A, Reynolds C F, Kupfer D J. (2000) Allelic variation in the serotonin transporter promoter affects onset of paroxetine treatment response in late-life depression. Neuropsychopharmacology 23, 587-90).

Inhibition of the serotonin transporter by paroxetine results in increases in synaptic serotonin concentration. This eventually results in downregulation (desensitization) of synaptic serotonin receptors autoreceptors1A and 1B/D (Roberts C, Boyd D F, Middlemiss D N, Routledge C. (1999) Enhancement of 5-HT1B and 5-HT1 D receptor antagonist effects on extracellular 5-HT levels in the guinea-pig brain following concurrent 5-HT1A or 5-HT re-uptake site blockade. Neuropharmacology 38, 1409-19; Roberts C, Price G W, Jones B J. (1997) The role of 5-HT(1B/1D) receptors in the modulation of 5-hydroxytryptamine levels in the frontal cortex of the conscious guinea pig. Eur J Pharmacol 326, 23-30; Davidson C, Stamford J A. (1997) Synergism of 5-HT 1B/D antagonists with paroxetine on serotonin efflux in rat ventral lateral geniculate nucleus slices. Brain Res Bull 43, 405-9; Barton C L, Hutson P H. (1999) Inhibition of hippocampal 5-HT synthesis by fluoxetine and paroxetine: evidence for the involvement of both 5-HT1A and 5-HT1B/D autoreceptors. Synapse 31, 13-9). The time for this adaptive change to occur underlies the delayed (4-8 weeks) therapeutic effect of SSRIs in major depression (Blier P, Pineyro G, el Mansari M, Bergeron R, de Montigny C. (1998) Role of somatodendritic 5-HT autoreceptors in modulating 5-HT neurotransmission. Ann N Y Acad Sci 861, 204-16). Definitive implication of these receptors in the alleviation of depressive symptoms via paroxeteine administration has been shown by studies where 5-HT1A and 5-HT1B/D receptor agonists attenuate the antidepressant activity of paroxetine (Bourin M, Redrobe J P, Baker G B. (1998) Pindolol does not act only on 5-HT1A receptors in augmenting antidepressant activity in the mouse forced swimming test. Psychopharmacology (Berl) 136, 226-34), and 5-HT1A and 1B/D receptors antagonists potentiate paroxetine's antidepressant activity (Blier P, Bergeron R, de Montigny C. (1997) Selective activation of postsynaptic 5-HT1A receptors induces rapid antidepressant response. Neuropsychopharmacology 16, 333-8; Tome M B, Isaac M T, Harte R, Holland C. (1997) Paroxetine and pindolol: a randomized trial of serotonergic autoreceptor blockade in the reduction of antidepressant latency. Int Clin Psychopharmacol 12, 81-9; Malagie I, Trillat A C, Bourin M, Jacquot C, Hen R, Gardier A M. (2001) 5-HT1B Autoreceptors limit the effects of selective serotonin re-uptake inhibitors in mouse hippocampus and frontal cortex. J Neurochem 76, 865-71). The use of receptor antagonists has been investigated as a methodology to be used during the early stages of SSRI treatment before the receptors have had time to effectively downregulate/desensitize (Zanardi R, Artigas F, Franchini L, Sforzini L, Gasperini M, Smeraldi E, Perez J. (1997) How long should pindolol be associated with paroxetine to improve the antidepressant response? J Clin Psychopharmacol 17, 446-50). These studies implicate the 5-HT1A and 1B/D autoreceptor groups as important targets to be included in our predictive algorithm.

Of the remaining serotonin receptor sub-classes, 5-HT2A has been shown to be primarily involved in collateral side effects (Pullar I A, Carney S L, Colvin E M, Lucaites V L, Nelson D L, Wedley S. (2000) LY367265, an inhibitor of the 5-hydroxytryptamine transporter and 5-hydroxytryptamine(2A) receptor antagonist: a comparison with the antidepressant, nefazodone. Eur J Pharmacol 407, 39-46; Sargent P A, Williamson D J, Cowen P J. (1998) Brain 5-HT neurotransmission during paroxetine treatment. Br J Psychiatry 172, 49-52), although it has been postulated to play a pivotal role in the anxiolytic effects of paroxetine (Schreiber R, Melon C, De Vry J. (1998) The role of 5-HT receptor subtypes in the anxiolytic effects of selective serotonin reuptake inhibitors in the rat ultrasonic vocalization test. Psychopharmacology (Berl) 135, 383-91). For this reason it is considered a secondary candidate. 5-HT3/4/5/6/7 (and subclasses) do not have reports in the literature regarding interaction with paroxetine in modulation of antidepressant activity and are not genes we intend to target.

Noradrenergic System

Paroxetine, as an SSRI, is widely portrayed as producing its therapeutic effects primarily by acting as a highly selective antagonist of the serotonin transporter. However, both in vitro (Owens M J, Morgan W N, Plott S J, Nemeroff C B. (1997) Neurotransmitter receptor and transporter binding profile of antidepressants and their metabolites. J Pharmacol Exp Ther 283, 1305-22) and in vivo (Owens M J, Knight D L, Nemeroff C B. (2000) Paroxetine binding to the rat norepinephrine transporter in vivo. Biol Psychiatry 47, 842-5) data indicate that paroxetine also inhibits the norepinephrine transport protein (NET). This data is consistent with reports that selective serotonin reuptake inhibitors affect the norepinephrine system (Blier P. (2001) Crosstalk between the norepinephrine and serotonin systems and its role in the antidepressant response. J Psychiatry Neurosci 26 Suppl, S3-10) and that paroxetine increases norepinephrine concentrations (Millan M J, Lejeune F, Gobert A. (2000) Reciprocal autoreceptor and heteroreceptor control of serotonergic, dopaminergic and noradrenergic transmission in the frontal cortex: relevance to the actions of antidepressant agents. J Psychopharmacol 14, 114-38; Carlson J N, Visker K E, Nielsen D M, Keller R W, Jr., Glick S D. (1996) Chronic antidepressant drug treatment reduces turning behavior and increases dopamine levels in the medial prefrontal cortex. Brain Res 707,122-6).

The alpha-2a-adrenergic autoreceptor is a primary regulator of NE release (Millan M J, Lejeune F, Gobert A. (2000) Reciprocal autoreceptor and heteroreceptor control of serotonergic, dopaminergic and noradrenergic transmission in the frontal cortex: relevance to the actions of antidepressant agents. J Psychopharmacol 14, 114-38). Inhibition of NET by paroxetine might be expected to desensitize the inhibitory alpha-2-adrenergic autoreceptors in a manner similar that reported for the 5-HT1A and 1B/D autoreceptor system under SERT inhibition. However, studies of venlafaxine, a preferential serotonin/norepinephrine transport protein inhibitor show that after chronic adminstration, the alpha-2-adrenergic receptors are not desensitized (Beique J, de Montigny C, Blier P, Debonnel G. (2000) Effects of sustained administration of the serotonin and norepinephrine reuptake inhibitor venlafaxine: II. In vitro studies in the rat. Neuropharmacology 39, 1813-22). This could lead to the hypothesis that they continue to regulate the levels of norepinephrine in the synapse (in which the 5-HT1A/B/D are attenuated after they desensitize) and that inhibition of the inhibition of NET is compensated for by the inhibitory action of the alpha-2a-adrenergic autoreceptor. It has been reported, however, that levels of norepinephrine levels increase with paroxetine treatment (Carlson J N, Visker K E, Nielsen D M, Keller R W, Jr., Glick S D. (1996) Chronic antidepressant drug treatment reduces turning behavior and increases dopamine levels in the medial prefrontal cortex. Brain Res 707, 122-6). Because alpha-2a-adrenergic is an inhibitory autoreceptor we believe it likely to be involved in the response mechanism in some manner, if not in a similar manner to that of the 5-HT autoreceptors (i.e., desensitization). This mechanism, however, has not been identified in the literature. For this reason we include it as a secondary candidate.

While the 5-HT1A receptors were implicated in the mechanism of action using the 5-HT1A antagonist pindolol (simulating receptor down regulation before it has time to occur secondary to inhibition of the serotonin transporter), selective antagonism of the beta-adrenergic receptors with metoprolol shows no increase in efficacy of treatment during the latent period (Zanardi R, Artigas F, Franchini L, Sforzini L, Gasperini M, Smeraldi E, Perez J. (1997) How long should pindolol be associated with paroxetine to improve the antidepressant response? J Clin Psychopharmacol 17, 446-50). Other findings, however, have indicated that the beta-adrenergic receptor is downregulated in response to chronic treatment with other SSRIs (indicating desensitization), but upregulated in depression models (Asakura M, Nagashima H, Fujii S, Sasuga Y, Misonoh A, Hasegawa H, Osada K. (2000) [Influences of chronic stress on central nervous systems]. Nihon Shinkei Seishin Yakurigaku Zasshi 20, 97-105).

Activation of the beta-2-adrenergic receptor has been reported to potentiate transactivation of glucocorticoid response elements via the glucocorticoid receptor (GR) (Schmidt P, Holsboer F, Spengler D. (2001) Beta(2)-adrenergic receptors potentiate glucocorticoid receptor transactivation via G protein beta gamma-subunits and the phosphoinositide 3-kinase pathway. Mol Endocrinol 15, 553-64). The GR has been shown to play a role as regulatory inhibitor in the HPA axis. Hypoactivty of GR resulting in hyperactivity of the HPA axis has been implicated in depression. Paroxetine increases levels of GR via transcriptional upregulation. Potentiation of the activity of GR via the beta-2-adrenergic receptor may be an important component in the alleviation of depression. Although the role of the beta-2-adrenergic receptor in depression and response to paroxetine in alleviation of depressive symptoms has not been conclusively isolated, we believe there is evidence indicating potential involvement.

Dopaminergic System

The dopaminergic system's participation in the etiology of depression is documented (Delgado P. (2000) Depression: the case for a monoamine deficiency. J Clin Psychiatry 61, 7-11), however, information on the effect of paroxetine on its activity is limited.

There is a report that dopamine release is facilitated by serotonin (Zangen A, Nakash R. Overstreet D H, Yadid G. (2001) Association between depressive behavior and absence of serotonin-dopamine interaction in the nucleus accumbens. Psychopharmacology (Berl) 155, 434-9). This potentially indicates an indirect effect of paroxetine (via increased levels of serotonin in the synapse). This is supported by the observation that paroxetine treatment increases levels of dialyzable dopamine (Millan M J, Lejeune F, Gobert A. (2000) Reciprocal autoreceptor and heteroreceptor control of serotonergic, dopaminergic and noradrenergic transmission in the frontal cortex: relevance to the actions of antidepressant agents. J Psychopharmacol 14, 114-38). Interestingly, dopamine levels are also increased in cases of NET inhibition (Carboni E, Tanda G L, Frau R, Di Chiara G. (1990) Blockade of the noradrenaline carrier increases extracellular dopamine concentrations in the prefrontal cortex: evidence that dopamine is taken up in vivo by noradrenergic terminals. J Neurochem 55, 1067-70). These data argue that the dopaminergic system is affected by treatment with paroxetine, however, in both cases, the mode of action (i.e., whether through inhibition of the dopamine transport protein or through D2 autoreceptor antagonism, or other) has not been defined. Paroxetine has been reported to have only very weak effects on the dopamine transport protein.

The only significant direct correlation of the dopaminergic system with paroxetine treatment is a report which correlates D1/D2 receptor responsivity with rapidity and success of response (Healy E, McKeon P. (2000) Dopaminergic sensitivity and prediction of antidepressant response. J Psychopharmacol 14, 152-6).

Based on the data available is appears that the strongest choice for gene selection are the D1 and D2 receptors. The dopamine transport protein may have indirect effects, however, there are no reports yet that correlate it with response to paroxetine.

Hypothalamic-Pituitary-Adrenal (HPA) Axis

Glucocorticoid receptor (GR) activation by glucocorticoids, with subsequent binding to and activation of the glucocorticoid responsive element, has been shown to be necessary component of the cortisol feedback loop of the hypothalamus-pituitary-adrenal (HPA) axis (Spencer R L, Kim P J, Kalman B A, Cole M A. (1998) Evidence for mineralocorticoid receptor facilitation of glucocorticoid receptor-dependent regulation of hypothalamic-pituitary-adrenal axis activity. Endocrinology 139, 2718-26; De Kloet E R, Vreugdenhil E, Oitzl M S, Joels M. (1998) Brain corticosteroid receptor balance in health and disease. Endocr Rev 19, 269-301). Abnormalities that result in attenuation of GR functionality and/or levels have been proposed to underlie hyperactivity of the HPA axis as described in patients with major depression (Pariante C M, Miller A H. (2001) Glucocorticoid receptors in major depression: relevance to pathophysiology and treatment. Biol Psychiatry 49, 391-404; Modell S, Yassouridis A, Huber J, Holsboer F. (1997) Corticosteroid receptor function is decreased in depressed patients. Neuroendocrinology 65, 216-22). Transgenic mice with disturbed GR function are reported to display several characteristics seen in depressive illness, including a hyperactive HPA axis (Barden N, Stec I S, Montkowski A, Holsboer F, Reul J M. (1997) Endocrine profile and neuroendocrine challenge tests in transgenic mice expressing antisense RNA against the glucocorticoid receptor. Neuroendocrinology 66, 212-20). Paroxetine has been demonstrated to increase levels of GR through transcriptional upregulation (Okugawa G, Omori K, Suzukawa J, Fujiseki Y, Kinoshita T, Inagaki C. (1999) Long-term treatment with antidepressants increases glucocorticoid receptor binding and gene expression in cultured rat hippocampal neurones. J Neuroendocrinol 11, 887-95), which has been proposed to restore glucocorticoid function (McQuade R, Young A H. (2000) Future therapeutic targets in mood disorders: the glucocorticoid receptor. Br J Psychiatry 177, 390-5).

Potential gene product candidates for analysis include factors involved in the transcriptional regulation of GR. These, however, have not yet been identified.

Study Population

First-time Estonian depressives were used for a prospective study of Paroxetine (Paxil) response to depression. The population of Estonia has been previously been identified as being consistent with the Caucasian population of other Northern European countries. The subject inclusion criterion was a Hamilton Depression Rating Scale (HAM-D) score of 18 or greater. The number of subjects enrolled was 203.

The data for this study was comprised of 203 subjects that were treated for depression with Paroxetine (Paxil). The severity of the depression of the subjects was assessed from the perspective of the patients and their physicians utilizing the HAM-D and CGI-S tests. The HAM-D assessment was performed five times, at presentation and on four follow-ups at two-week intervals.

The patients were selected for study inclusion by satisfying intake criteria of a HAM-D greater or equal to 18 in order to select a depressed patient pool. This created a pool of subjects that were expected to be significantly depressed, but introduced an artifact in the study for evaluating outcome.

Outcome Measure-Paroxetine

As with the citalopram study, due to selection criteria, the relationship between the HAM-D and CGI-S scores of week 0 are significantly different from those in weeks 2-8. The Spearman correlation coefficient between HAM-D and CGI-S for subjects at weeks 2, 4, 6 and 8 are 0.8, 0.8, 0.8 and 0.7, respectively, with an overall coefficient of 0.8. The composite linear regression coefficient with a forced origin of 0 between HAM-D and CGI-S for weeks 2-8 was 4.0, as shown in equation 1. However because of the imposed patient recruitment criteria, the Spearman correlation coefficient between HAM-D and CGI-S for week 0 is only 0.5. This induced bias can be easily observed in FIG. 4, which illustrates the correspondence between the HAM-D and CGI-S measures for the included subjects in weeks 0, 2, 4, 6, and 8.

Because of this induced bias, HAM-D scores from week 0 were not used to assess patient response to the study protocol. In response, an outcome measure was devised to account for both the patient and the physician report of the severity of the depression that excluded the HAM-D from week 0. Outcome measure Y is the averaged weighted CGI-S and HAM-D score of the 8^{th} week normalized by an averaged weighted CGI-S and HAM-D score of the 0^{th} and 2^{nd} weeks, respectively as stated in equation 2. The CGI-S scores were weighted by a factor of 4 for equalization with HAM-D scores.

In order to increase separation between the classes of responding and non-responding subjects, the outcome measure used for model training was the product of Y8/Y0 and Y8, as given in equation 3. The threshold in the outcome measure Y of 4.0 was selected to separate responders from non-responders.
HAM-D≈0.0+CGI-S*4.0 (5)
Y₀=(2*CGI-S(wk0)*4.0+HAM-D₁₇(wk2))/3 (6)
Y₈=(CGI-S(wk8)*4.0+HAM-D₁₇(wk8))/2 (7)
Y=Y₀*Y₀/Y₈ (8)

Feature Subset Selection

An initial allele pool for model development was selected by filtering the total allele pool with a Kruskal-Wallis test for significance using the thresholded outcome measure y>4 as a group indicator. The Kruskal-Wallis test is a nonparametric version of one-way analysis of variance. The assumption behind this test is that the measurements come from a continuous distribution, but not necessarily a normal distribution. The test is based on an analysis of variance using the ranks of the data values, not the data values itself.

The goal was to select a set of relevant features from the complete set of SNPs that resulted in a predictive relationship for response or non-response to Paroxetine.

Feature selection and model parameterization were performed jointly with 10 fold cross validated Naïve testing and 10 fold cross validation on the training utilizing custom algorithms developed by Prediction Sciences. This procedure consists of forming 10 nearly disjoint testing sets and then for each of these testing sets forming 10 training/cross-validation sets. Data stratification is maintained with respect to the outcome measure while forming these sets so there is roughly equal representation of the original data set outcome distribution in each of the individual training, cross-validation, and Naïve testing sets.

Feature Selection

We found for this data set that feature selection was best performed using a cross-validated tree method. The trees were initialized from a subset of the total population of a binary allele feature set. Each of 10 training sets was partitioned into 10 sub-samples, chosen randomly but with roughly equal size and roughly the same class proportions. For each training/cross validation set, a classification tree was fit to the training data and used to predict the response category for the cross-validation set.

The trees were trained with Gini's diversity index for the split criterion. The cost function utilized for optimization is described by a square matrix C, C_{i,j}, which is the cost of classifying a point into class i if its true class is j. A typical cost function would be the identity matrix (C_{i,j}=1 if i≅j, and C_{i,j}=1 if i=j). However, for this modeling process, the identity matrix failed to result in a converging model set with forward feature selection. Therefore, an alternative cost matrix C was specified.

$$C = \begin{matrix}0 & 1 & 2 & 3 & 4 \\ 1 & 0 & 2 & 3 & 4 \\ 7 & 5 & 0 & 1 & 2 \\ 7 & 5 & 1 & 0 & 1 \\ 7 & 6 & 2 & 1 & 0\end{matrix} \qquad ( 9 )$$

This cost function strongly penalized false negatives and to a lesser degree penalized false positives. This approach was selected as it was hypothesized that polymorphisms in the relevant proteins would be more likely to interfere with the action of the therapeutic compound rather than enhancing the action of the therapeutic compound on alleviating depression.

After model parameterization and feature selection, pruning was performed in order to select the model structure that best identified responders and non-responders by maximizing the cross validation cost function. This prevented overtraining, increasing the opportunity for generalization on naïve data. The information from all cross-validation sets was use jointly to compute the model prediction cost as a function of pruning level. For each of the 10 train/test sets, the pruning level that minimized the cross-validation cost function was selected as the model order for the corresponding tree.

Post Processing

Finally, after model completion, the degree of representation for each of the bins from each of the models was assessed, and bins 4 and 5 were combined as bin 5 was not well populated and their predictive performance was not statistically different, as assessed by the percentage of subjects with an outcome measure Y of less than 50%.

The finalized models were then applied to their corresponding Naïve test sets (i.e. completely new patient data) to check for statistical consistency in feature selection and model parameterization.

The SNPs selected based on this joint selection and model parameterization method for the ten data-model sets and the corresponding frequencies of the SNPS in the model sets is given in FIG. 8.

The naïve testing performance for complete data, missing data, and the composite data set spanning the complete set of subjects is given in FIG. 9.

In total, 29 SNPs were included in the ten sub-models of the constructed model set. Three SNPs were included in 8 or more of the models, which are in genes coding for the SLC6A4 solute carrier family 6 (neurotransmitter transporter, serotonin), member 4, HTR1A 5-hydroxytryptamine (serotonin) receptor 1A, and ATP-binding cassette, sub-family B (MDR/TAP), member 1. Three SNPs were included in 5 or 6 of the models:

ATP-binding cassette, sub-family B (MDR/TAP), member 1, DRD2 dopamine receptor D2, and ATP-binding cassette, sub-family B (MDR/TAP), member 1. Of the remaining 23 SNPS selected, an additional 3 code for dopamine receptor D2, and 5 code for HTR1A 5-hydroxytryptamine (serotonin) receptors.

Of the commonly identified SNPS in the ten models, none strongly decreased the probability of response as hypothesized. With low predictive sensitivity SNPs rs2242592 (DRD2 dopamine receptor D2), and rs42460 (UD, SLC6A4 solute carrier family 6 (neurotransmitter transporter, serotonin), member 4) decreased or slightly decreased the probability of response to 55% (specificity 0.94) and 68% (specificity >0.95), respectively. In contrast, SNPs 2235048, (ATP-binding cassette, sub-family B (MDR/TAP), member 1), and 2235015 (ATP-binding cassette, sub-family B (MDRFTAP), member 1) increased the probability of response to 0.88, with specificities of 0.92 and 0.95, respectively.

However, as all of these alleles provide only low predictive sensitivity, there is a need for a means of combining the features to produce a model with lower false negative rates and greater applicability.

The models produced from the forward feature selection to maximize negative predictive power and minimize false negative rate were able to successfully identify patients with increased and decreased probabilities of response compared to the average population response rates in the study. The model produces four bins (1-4) as shown in table 3. Bin 1 corresponds to responding subjects and has a false positive rate on naïve data of <20%. Bin 2 corresponds to subjects that had a probability of response of slightly better than average. Bin 3 corresponds to increased likelihood of non-response, the group having a probability of response of approximately 60%. Bin 4 corresponds to non-responders with a probability of response of <50%.

Secondary Modeling Approach

Due to the broad multigenic nature of the identified SNPs in the initial modeling results, an alternative modeling methodology was employed with another added preprocessing step. A new interaction data set was formed from the original coded binary allele data set. The new data set was composed of positive-positive indications, and a positive negative indication for all possible allele pairings, as well as the original individual coded alleles. Features to be included in the first stage of feature selection were identified from this expanded set by testing for whether the outcome measure grouped each column of the expanded set appeared to have been drawn from a statistically different population p<0.05 as assessed by the Kruskal-Wallis test. This provided a broad set of features to assess in a secondary selection step. This initial subset of features was then screened according to a positive or negative prediction value normalized by the prevalence of the positive or negative indication, respectively. Features in the top 10 percent of the resulting distribution were accepted for use in modeling. This resulted in 508 features to assess for citalopram and 892 to assess for Paroxetine. The modeling was accomplished by SFFS in conjunction with a probabilistic neural network. The probabilistic neural network is a class of a radial basis network that is suitable for classification. The only parameter that is ad hoc is the spread of the radial basis function. This parameter was optimized in 5 fold cross validation loop.

The cost function utilized for features selection was a function of the prediction, the truth, and a cost matrix. The prediction and truth outcomes were based on 5 classes derived by labeling the continuous outcome measure as one of 5 bins. Bins 1 and 2 correspond to responders. Bin 3 corresponds to marginal non-responders, and Bins 4 and 5 correspond to clear non-responders. This was done due to the inherent noise in the outcome measure and the choice of the modeling method employed.

The cost matrix utilized for during this training the trees is described by a square matrix C=Cᵢⱼ, which is the cost of classifying a point into class i (row i) if its true class is j (column j). A typical cost function would be the identity matrix (Cᵢⱼ=1 if i≈j, Cᵢⱼ=0 if i=j). The cost matrix C was specified for both citalopram and paroxetine is given in equation 10.

$$C = \begin{matrix}0 & 0 & 3 & 4 & 5 \\ 0 & 0 & 2 & 3 & 4 \\ 5 & 4 & 0 & 1 & 2 \\ 7 & 6 & .5 & 0 & .5 \\ 9 & 8 & 1 & .5 & 0\end{matrix} \qquad ( 10 )$$

SFFS feature selection proceeded for 60 generations, with a maximum of 4 additions and 4 subtractions at each generation. At each step, the cross-validation sets were scored and used jointly to compute the model prediction cost. This is the cost used to identify features for inclusion or exclusion. As long as the cost decreased with feature inclusion, then up to 4 features could be added. Afterward, as many as 4 features could be removed if the cost decreased or remained the same with feature removal.

The probabilistic net models for citalopram and paroxetine were trained to predict the probability to respond to treatment in discrete levels, with level 1 being the best chance of response and level 5 the least. However, for both models after training, levels 4 and 5 were combined as level 5 was not well populated and the predictive performance of level 5 was not statistically different from level 4, as assessed by the percentage of subjects with an outcome measure Y of less than 50%.

After modeling was complete, the impact of removing SNPs that occurred in only one of the submodels was assessed on the cross-validation model performance. The increase in total cost was assessed by computing the data matrix with each of the singleton snps set as missing. The missing values propagated through the allele coding and interaction set formation, and the corresponding features were set equal to the mean value of their corresponding training data. The performance cost was then computed with each of the singleton SNPs effectively removed one at a time using the cost matrix of equation 10. The result was a list of marginal contributions to the final model performance of each of the selected SNPs. Those SNPs with low marginal contributions were then formed into a set, and the full set was removed simultaneously to assess the impact on cross-validation model performance. As expected, the impact was minor and the final naïve model testing performance was assessed with the low impact SNPs set as missing.

The SNPs selected based on the secondary modeling joint feature selection and probabilistic neural net training method for the four (five for paroxetine) data-model sets most predictive of outcome and the corresponding frequencies of the SNPs in the model set is given in Table 12 for citalopram, and Table 10 for paroxetine.

The naïve testing performance for all, complete and missing data with the secondary model set spanning the complete set of subjects is given in FIG. 13 for citalopram, and FIG. 11 for paroxetine.

Diagnostic Detection of Depression Disease-Associated and Treatment-Relevant Mutations:

According to the present invention, base changes in the genes can be detected and used as a diagnostic for Depression. A variety of techniques are available for isolating DNA and RNA and for detecting mutations in the isolated ABCB1, ABCB4, ADRA1A, ADRA1D, ADRA2A, ADRB1, ADRB2, COMT, CRH, CRHBP, CRHR1, CRHR2, CYP2D6, CYP3A4, CYP2D19, DRD1, DRD2, DRD3, DRD4, GLRF1, HTR1A, HTR1B, HTR1D, HTR2A, HTR2B, HTR2C, HTR3A, HTR3B, MAOA, MAOB, NR3C1, SLC6A2 SLC6A3, SLC6A4, TAC1, TACR1 or TPH genes.

A number of sample preparation methods are available for isolating DNA and RNA from patient blood samples. For example, the DNA from a blood sample is obtained by cell lysis following alkali treatment. Often, there are multiple copies of RNA message per DNA. Accordingly, it is useful from the standpoint of detection sensitivity to have a sample preparation protocol which isolates both forms of nucleic acid. Total nucleic acid may be isolated by guanidium isothiocyanate/phenol-chloroform extraction, or by proteinase K/phenol-chloroform treatment. Commercially available sample preparation methods such as those from Qiagen Inc. (Chatsworth, Calif.) can also be utilized.

As discussed more fully hereinbelow, hybridization with one or more labelled probes containing complements of the variant sequences enables detection of the Depression mutations. Since each Depression patient can be heteroplasmic (possessing both the Depression mutation and the normal sequence) a quantitative or semi-quantitative measure (depending on the detection method) of such heteroplasmy can be obtained by comparing the amount of signal from the Depression probe to the amount from the Depression.sup.—(normal or wild-type) probe.

A variety of techniques, as discussed more fully hereinbelow, are available for detecting the specific mutations in the ABCB1, ABCB4, ADRA1A, ADRA1D, ADRA2A, ADRB1, ADRB2, COMT, CRH, CRHBP, CRHR1, CRHR2, CYP2D6, CYP3A4, CYP2D19, DRD1, DRD2, DRD3, DRD4, GLRF1, HTR1A, HTR1B, HTR1D, HTR2A, HTR2B, HTR2C, HTR3A, HTR3B, MAOA, MAOB, NR3C1, SLC6A2 SLC6A3, SLC6A4, TAC1, TACR1 or TPH genes. The detection methods include, for example, cloning and sequencing, ligation of oligonucleotides, use of the polymerase chain reaction and variations thereof, use of single nucleotide primer-guided extension assays, hybridization techniques using target-specific oligonucleotides and sandwich hybridization methods.

Cloning and sequencing of the ABCB1, ABCB4, ADRA1A, ADRA1D, ADRA2A, ADRB1, ADRB2, COMT, CRH, CRHBP, CRHR1, CRHR2, CYP2D6, CYP3A4, CYP2D19, DRD1, DRD2, DRD3, DRD4, GLRF1, HTR1A, HTR1B, HTR1D, HTR2A, HTR2B, HTR2C, HTR3A, HTR3B, MAOA, MAOB, NR3C1, SLC6A2 SLC6A3, SLC6A4, TAC1, TACR1 or TPH genes can serve to detect Depression mutations in patient samples. Sequencing can be carried out with commercially available automated sequencers utilizing fluorescently labelled primers. An alternate sequencing strategy is the “sequencing by hybridization” method using high density oligonucleotide arrays on silicon chips (Fodor et al., Nature 364:555-556 (1993); Pease et al., Proc. Natl. Acad. Sci. USA, 91:5022-5026 (1994). For example, fluorescently-labelled target nucleic acid generated, for example from PCR amplification of the target genes using fluorescently labelled primers, are hybridized with a chip containing a set of short oligonucleotides which probe regions of complementarity with the target sequence. The resulting hybridization patterns are useful for reassembling the original target DNA sequence.

Mutational analysis can also be carried out by methods based on ligation of oligonucleotide sequences which anneal immediately adjacent to each other on a target DNA or RNA molecule (Wu and Wallace, Genomics 4:560-569 (1989); Landren et al., Science 241:1077-1080 (1988); Nickerson et al., Proc. Natl. Acad. Sci. 87:8923-8927 (1990); Barany, F., Proc. Natl. Acad. Sci. 88:189-193 (1991)). Ligase-mediated covalent attachment occurs only when the oligonucleotides are correctly base-paired. The Ligase Chain Reaction (LCR), which utilizes the thermostable Taq ligase for target amplification, is particularly useful for interrogating Depression mutation loci. The elevated reaction temperatures permits the ligation reaction to be conducted with high stringency (Barany, F., PCR Methods and Applications 1:5-16 (1991)).

Analysis of point mutations in DNA can also be carried out by using the polymerase chain reaction (PCR) and variations thereof. Mismatches can be detected by competitive oligonucleotide priming under hybridization conditions where binding of the perfectly matched primer is favored (Gibbs et al., Nucl. Acids. Res. 17:2437-2448 (1989)). In the amplification refractory mutation system technique (ARMS), primers are designed to have perfect matches or mismatches with target sequences either internal or at the 3′ residue (Newton et al., Nucl. Acids. Res. 17:2503-2516 (1989)). Under appropriate conditions, only the perfectly annealed oligonucleotide functions as a primer for the PCR reaction, thus providing a method of discrimination between normal and mutant (Depression) sequences.

Genotyping analysis of the ABCB1, ABCB4, ADRA1A, ADRA1D, ADRA2A, ADRB1, ADRB2, COMT, CRH, CRHBP, CRHR1, CRHR2, CYP2D6, CYP3A4, CYP2D19, DRD1, DRD2, DRD3, DRD4, GLRF1, HTR1A, HTR1B, HTR1D, HTR2A, HTR2B, HTR2C, HTR3A, HTR3B, MAOA, MAOB, NR3C1, SLC6A2 SLC6A3, SLC6A4, TAC1, TACR1 or TPH genes can also be carried out using single nucleotide primer-guided extension assays, where the specific incorporation of the correct base is provided by the high fidelity of the DNA polymerase (Syvanen et al., Genomics 8:684-692 (1990); Kuppuswamy et al., Proc. Natl. Acad. Sci. USA. 88:1143-1147 (1991)). Another primer extension assay, which allows for the quantification of heteroplasmy by simultaneously interrogating both wild-type and mutant nucleotides, is disclosed in a co-pending U.S. patent application entitled, “Multiplexed Primer Extension Methods”, naming Eoin Fahy and Soumitra Ghosh as inventors, filed on Mar. 24, 1995, Ser. No. 08/410,658, the disclosure of which is incorporated by reference.

Detection of single base mutations in target nucleic acids can be conveniently accomplished by differential hybridization techniques using target-specific oligonucleotides (Suggs et al., Proc. Natl. Acad. Sci. 78:6613-6617 (1981); Conner et al., Proc. Natl. Acad. Sci. 80:278-282 (1983); Saiki et al., Proc. Natl. Acad. Sci. 86:6230-6234 (1989)). For example, mutations are diagnosed on the basis of the higher thermal stability of the perfectly matched probes as compared to the mismatched probes. The hybridization reactions may be carried out in a filter-based format, in which the target nucleic acids are immobilized on nitrocellulose or nylon membranes and probed with oligonucleotide probes. Any of the known hybridization formats may be used, including Southern blots, slot blots, “reverse” dot blots, solution hybridization, solid support based sandwich hybridization, bead-based, silicon chip-based and microtiter well-based hybridization formats.

An alternative strategy involves detection of the ABCB1, ABCB4, ADRA1A, ADRA1D, ADRA2A, ADRB1, ADRB2, COMT, CRH, CRHBP, CRHR1, CRHR2, CYP2D6, CYP3A4, CYP2D19, DRD1, DRD2, DRD3, DRD4, GLRF1, HTR1A, HTR1B, HTR1D, HTR2A, HTR2B, HTR2C, HTR3A, HTR3B, MAOA, MAOB, NR3C1, SLC6A2 SLC6A3, SLC6A4, TAC1, TACR1 or TPH genes by sandwich hybridization methods. In this strategy, the mutant and wild-type (normal) target nucleic acids are separated from non-homologous DNA/RNA using a common capture oligonucleotide immobilized on a solid support and detected by specific oligonucleotide probes tagged with reporter labels. The capture oligonucleotides can be immobilized on microtitre plate wells or on beads (Gingeras et al., J. Infect. Dis. 164:1066-1074 (1991); Richman et al., Proc. Natl. Acad. Sci. 88:11241-11245 (1991)).

While radio-isotopic labeled detection oligonucleotide probes are highly sensitive, non-isotopic labels are preferred due to concerns about handling and disposal of radioactivity. A number of strategies are available for detecting target nucleic acids by non-isotopic means (Matthews et al., Anal. Biochem., 169:1-25 (1988)). The non-isotopic detection method may be direct or indirect.

The indirect detection process is generally where the oligonucleotide probe is covalently labelled with a hapten or ligand such as digoxigenin (DIG) or biotin. Following the hybridization step, the target-probe duplex is detected by an antibody- or streptavidin-enzyme complex. Enzymes commonly used in DNA diagnostics are horseradish peroxidase and alkaline phosphatase. One particular indirect method, the Genius.™. detection system (Boehringer Mannheim) is especially useful for mutational analysis of the ABCB1, ABCB4, ADRA1A, ADRA1D, ADRA2A, ADRB1, ADRB2, COMT, CRH, CRHBP, CRHR1, CRHR2, CYP2D6, CYP3A4, CYP2D19, DRD1, DRD2, DRD3, DRD4, GLRF1, HTR1A, HTR1B, HTR1D, HTR2A, HTR2B, HTR2C, HTR3A, HTR3B, MAOA, MAOB, NR3C1, SLC6A2 SLC6A3, SLC6A4, TAC1, TACR1 or TPH genes. This indirect method uses digoxigenin as the tag for the oligonucleotide probe and is detected by an anti-digoxigenin-antibody-alkaline phosphatase conjugate.

Direct detection methods include the use of fluorophor-labeled oligonucleotides, lanthanide chelate-labeled oligonucleotides or oligonucleotide-enzyme conjugates. Examples of fluorophor labels are fluorescein, rhodamine and phthalocyanine dyes. Examples of lanthanide chelates include complexes of Eu.sup.3+ and Tb.sup.3+. Directly labeled oligonucleotide-enzyme conjugates are preferred for detecting point mutations when using target-specific oligonucleotides as they provide very high sensitivities of detection.

Oligonucleotide-enzyme conjugates can be prepared by a number of methods (Jablonski et al., Nucl. Acids Res., 14:6115-6128 (1986); Li et al., Nucl. Acids Res. 15:5275-5287 (1987); Ghosh et al., Bioconjugate Chem. 1:71-76 (1990)), and alkaline phosphatase is the enzyme of choice for obtaining high sensitivities of detection. The detection of target nucleic acids using these conjugates can be carried out by filter hybridization methods or by bead-based sandwich hybridization (Ishii et al., Bioconjugate Chemistry 4:34-41 (1993)).

Detection of the probe label may be accomplished by the following approaches. For radioisotopes, detection is by autoradiography, scintillation counting or phosphor imaging. For hapten or biotin labels, detection is with antibody or streptavidin bound to a reporter enzyme such as horseradish peroxidase or alkaline phosphatase, which is thendetected by enzymatic means. For fluorophor or lanthanide-chelate labels, fluorescent signals may be measured with spectrofluorimeters with or without time-resolved mode or using automated microtitre plate readers. With enzyme labels, detection is by color or dye deposition (p-nitropheny phosphate or 5-bromo-4-chloro-3-indolyl phosphate/nitroblue tetrazolium for alkaline phosphatase and 3,3′-diaminobenzidine-NiCl.sub.2 for horseradish peroxidase), fluorescence (e.g., 4-methyl umbelliferyl phosphate for alkaline phosphatase) or chemiluminescence (the alkaline phosphatase dioxetane substrates LumiPhos 530 from Lumigen Inc., Detroit Mich. or AMPPD and CSPD from Tropix, Inc.). Chemiluminescent detection may be carried out with X-ray or polaroid film or by using single photon counting luminometers. This is the preferred detection format for alkaline phosphatase labelled probes.

The oligonucleotide probes for detection preferably range in size between 10 and 100 bases, more preferably between 15 and 30 bases in length. Examples of such nucleotide probes are found below in Tables 4 and 5. Tables 5 and 6 provide representative sequences of probes for detecting mutations in ABCB1, ABCB4, ADRA1A, ADRA1D, ADRA2A, ADRB1, ADRB2, COMT, CRH, CRHBP, CRHR1, CRHR2, CYP2D6, CYP3A4, CYP2D19, DRD1, DRD2, DRD3, DRD4, GLRF1, HTR1A, HTR1B, HTR1D, HTR2A, HTR2B, HTR2C, HTR3A, HTR3B, MAOA, MAOB, NR3C1, SLC6A2 SLC6A3, SLC6A4, TAC1, TACR1 or TPH genes and representative antisense sequences. In order to obtain the required target discrimination using the detection oligonucleotide probes, the hybridization reactions are preferably run between 20.degree. C. and 60.degree. C., and more preferably between 30.degree. C. and 55.degree. C. As known to those skilled in the art, optimal discrimination between perfect and mismatched duplexes can be obtained by manipulating the temperature and/or salt concentrations or inclusion of formamide in the stringency washes.

As an alternative to detection of mutations in the nucleic acids associated with the ABCB1, ABCB4, ADRA1A, ADRA1D, ADRA2A, ADRB1, ADRB2, COMT, CRH, CRHBP, CRHR1, CRHR2, CYP2D6, CYP3A4, CYP2D19, DRD1, DRD2, DRD3, DRD4, GLRF1, HTR1A, HTR1B, HTR1D, HTR2A, HTR2B, HTR2C, HTR3A, HTR3B, MAOA, MAOB, NR3C1, SLC6A2 SLC6A3, SLC6A4, TAC1, TACR1 or TPH genes, it is also possible to analyze the protein products of the ABCB1, ABCB4, ADRA1A, ADRA1D, ADRA2A, ADRB1, ADRB2, COMT, CRH, CRHBP, CRHR1, CRHR2, CYP2D6, CYP3A4, CYP2D19, DRD1, DRD2, DRD3, DRD4, GLRF1, HTR1A, HTR1B, HTR1D, HTR2A, HTR2B, HTR2C, HTR3A, HTR3B, MAOA, MAOB, NR3C1, SLC6A2 SLC6A3, SLC6A4, TAC1, TACR1 or TPH genes. In particular, point mutations in these genes are expected to alter the structure of the proteins for which these gene encode. These altered proteins (variant polypeptides) can be isolated and used to prepare antisera and monoclonal antibodies that specifically detect the products of the mutated genes and not those of non-mutated or wild-type genes. Mutated gene products also can be used to immunize animals for the production of polyclonal antibodies. Recombinantly produced peptides can also be used to generate polyclonal antibodies. These peptides may represent small fragments of gene products produced by expressing regions of the mitochondrial genome containing point mutations.

More particularly, variant polypeptides from point mutations in said genes can be used to immunize an animal for the production of polyclonal antiserum. For example, a recombinantly produced fragment of a variant polypeptide can be injected into a mouse along with an adjuvant so as to generate an immune response. Murine immunoglobulins which bind the recombinant fragment with a binding affinity of at least 1.times.10.sup.7 M.sup.-1 can be harvested from the immunized mouse as an antiserum, and may be further purified by affinity chromatography or other means. Additionally, spleen cells are harvested from the mouse and fused to myeloma cells to produce a bank of antibody-secreting hybridoma cells. The bank of hybridomas can be screened for clones that secrete immunoglobulins which bind the recombinantly produced fragment with an affinity of at least 1.times.10.sup.6 M.sup.-1. More specifically, immunoglobulins that selectively bind to the variant polypeptides but poorly or not at all to wild-type polypeptides are selected, either by pre-absorption with wild-type proteins or by screening of hybridoma cell lines for specific idiotypes that bind the variant, but not wild-type, polypeptides.

Nucleic acid sequences capable of ultimately expressing the desired variant polypeptides can be formed from a variety of different polynucleotides (genomic or cDNA, RNA, synthetic oligonucleotides, etc.) as well as by a variety of different techniques.

The DNA sequences can be expressed in hosts after the sequences have been operably linked to (i.e., positioned to ensure the functioning of) an expression control sequence. These expression vectors are typically replicable in the host organisms either as episomes or as an integral part of the host chromosomal DNA. Commonly, expression vectors can contain selection markers (e.g., markers based on tetracyclinic resistance or hygromycin resistance) to permit detection and/or selection of those cells transformed with the desired DNA sequences. Further details can be found in U.S. Pat. No. 4,704,362.

Polynucleotides encoding a variant polypeptide may include sequences that facilitate transcription (expression sequences) and translation of the coding sequences such that the encoded polypeptide product is produced. Construction of such polynucleotides is well known in the art. For example, such polynucleotides can include a promoter, a transcription termination site (polyadenylation site in eukaryotic expression hosts), a ribosome binding site, and, optionally, an enhancer for use in eukaryotic expression hosts, and, optionally, sequences necessary for replication of a vector.

E. coli is one prokaryotic host useful particularly for cloning DNA sequences of the present invention. Other microbial hosts suitable for use include bacilli, such as Bacillus subtilus, and other enterobacteriaceae, such as Salmonella, Serratia, and various Pseudomonas species. In these prokaryotic hosts one can also make expression vectors, which will typically contain expression control sequences compatible with the host cell (e.g., an origin of replication). In addition, any number of a variety of well-known promoters will be present, such as the lactose promoter system, a tryptophan (Trp) promoter system, a beta-lactamase promoter system, or a promoter system from phage lambda. The promoters will typically control expression, optionally with an operator sequence, and have ribosome binding site sequences, for example, for initiating and completing transcription and translation.

Other microbes, such as yeast, may also be used for expression. Saccharomyces can be a suitable host, with suitable vectors having expression control sequences, such as promoters, including 3-phosphoglycerate kinase or other glycolytic enzymes, and an origin of replication, termination sequences, etc. as desired.

In addition to microorganisms, mammalian tissue cell culture may also be used to express and produce the polypeptides of the present invention. Eukaryotic cells are actually preferred, because a number of suitable host cell lines capable of secreting intact human proteins have been developed in the art, and include the CHO cell lines, various COS cell lines, HeLa cells, myeloma cell lines, Jurkat cells, and so forth. Expression vectors for these cells can include expression control sequences, such as an origin of replication, a promoter, an enhancer, and necessary information processing sites, such as ribosome binding sites, RNA splice sites, polyadenylation sites, and transcriptional terminator sequences. Preferred expression control sequences are promoters derived from immunoglobulin genes, SV40, Adenovirus, Bovine Papilloma Virus, and so forth. The vectors containing the DNA segments of interest (e.g., polypeptides encoding a variant polypeptide) can be transferred into the host cell by well-known methods, which vary depending on the type of cellular host. For example, calcium chloride transfection is commonly utilized for prokaryotic cells, whereas calcium phosphate treatment or electroporation may be used for other cellular hosts.

The method lends itself readily to the formulation of test kits for use in diagnosis. Such a kit would comprise a carrier compartmentalized to receive in close confinement one or more containers wherein a first container may contain suitably labeled DNA or immunological probes. Other containers may contain reagents useful in the localization of the labeled probes, such as enzyme substrates. Still other containers may contain restriction enzymes, buffers etc., together with instructions for use.

Therapeutic Treatment of Depression:

Suppressing the effects of the mutations through antisense technology could provide an effective therapy for Depression. Much is known about ‘antisense’ therapies targeting messenger RNA (mRNA) or nuclear DNA. Hlen et al., Biochem. Biophys. Acta 1049:99-125 (1990). The diagnostic test of the present invention is useful for determining which of the specific Depression mutations exist in a particular Depression patient; this allows for “custom” treatment of the patient with antisense oligonucleotides only for the detected mutations. This patient-specific antisense therapy is also novel, and minimizes the exposure of the patient to any unnecessary antisense therapeutic treatment. As used herein, an “antisense” oligonucleotide is one that base pairs with single stranded DNA or RNA by Watson-Crick base pairing and with duplex target DNA via Hoogsteen hydrogen bonds. This also applies to gene silencing through sRNA as well.

The destructive effect of the Depression mutations in ABCB1, ABCB4, ADRA1A, ADRA1D, ADRA2A, ADRB1, ADRB2, COMT, CRH, CRHBP, CRHR1, CRHR2, CYP2D6, CYP3A4, CYP2D19, DRD1, DRD2, DRD3, DRD4, GLRF1, HTR1A, HTR1B, HTR1D, HTR2A, HTR2B, HTR2C, HTR3A, HTR3B, MAOA, MAOB, NR3C1, SLC6A2 SLC6A3, SLC6A4, TAC1, TACR1 or TPH genes is preferably reduced or eliminated using antisense oligonucleotide agents. Such antisense agents target DNA, by triplex formation with double-stranded DNA, by duplex formation with single-stranded DNA during transcription, or both. In a preferred embodiment, antisense agents target messenger RNA coding for the mutated ABCB1, ABCB4, ADRA1A, ADRA1D, ADRA2A, ADRB1, ADRB2, COMT, CRH, CRHBP, CRHR1, CRHR2, CYP2D6, CYP3A4, CYP2D19, DRD1, DRD2, DRD3, DRD4, GLRF1, HTR1A, HTR1B, HTR1D, HTR2A, HTR2B, HTR2C, HTR3A, HTR3B, MAOA, MAOB, NR3C1, SLC6A2 SLC6A3, SLC6A4, TAC1, TACR1 or TPH gene(s). Since the sequences of both the DNA and the mRNA are the same, it is not necessary to determine accurately the precise target to account for the desired effect. Procedures for inhibiting gene expression in cell culture and in vivo can be found, for example, in C. F. Bennett, et al. J. Liposome Res., 3:85 (1993) and C. Wahlestedt, et al. Nature, 363:260 (1993).

Antisense oligonucleotide therapeutic agents demonstrate a high degree of pharmaceutical specificity. This allows the combination of two or more antisense therapeutics at the same time, without increased cytotoxic effects. Thus, when a patient is diagnosed as having two or more Depression mutations in ABCB1, ABCB4, ADRA1A, ADRA1D, ADRA2A, ADRB1, ADRB2, COMT, CRH, CRHBP, CRHR1, CRHR2, CYP2D6, CYP3A4, CYP2D19, DRD1, DRD2, DRD3, DRD4, GLRF1, HTR1A, HTR1B, HTR1D, HTR2A, HTR2B, HTR2C, HTR3A, HTR3B, MAOA, MAOB, NR3C1, SLC6A2 SLC6A3, SLC6A4, TAC1, TACR1 or TPH genes, the therapy is preferably tailored to treat the multiple mutations simultaneously. When combined with the present diagnostic test, this approach to “patient-specific therapy” results in treatment restricted to the specific mutations detected in a patient. This patient-specific therapy circumvents the need for ‘broad spectrum’ antisense treatment using all possible mutations. The end result is less costly treatment, with less chance for toxic side effects.

One method to inhibit the synthesis of proteins is through the use of antisense or triplex oligonucleotides, analogues or expression constructs. These methods entail introducing into the cell a nucleic acid sufficiently complementary in sequence so as to specifically hybridize to the target gene or to mRNA. In the event that the gene is targeted, these methods can be extremely efficient since only a few copies per cell are required to achieve complete inhibition. Antisense methodology inhibits the normal processing, translation or half-life of the target message. Such methods are well known to one skilled in the art.

Antisense and triplex methods generally involve the treatment of cells or tissues with a relatively short oligonucleotide, although longer sequences can be used to achieve inhibition. The oligonucleotide can be either deoxyribo- or ribonucleic acid and must be of sufficient length to form a stable duplex or triplex with the target RNA or DNA at physiological temperatures and salt concentrations. It should also be sufficiently complementary or sequence specific to specifically hybridize to the target nucleic acid: Oligonucleotide lengths sufficient to achieve this specificity are preferably about 10 to 60 nucleotides long, more preferably about 10 to 20 nucleotides long. However, hybridization specificity is not only influenced by length and physiological conditions but may also be influenced by such factors as GC content and the primary sequence of the oligonucleotide. Such principles are well known in the art and can be routinely determined by one who is skilled in the art.

As an example, many of the oligonucleotide sequences used in connection with probes can also be used as antisense agents, directed to either the DNA or resultant messenger RNA.

A great range of antisense sequences can be designed for a given mutation. Oligonucleotide sequences can be easily designed by one of ordinary skill in the art to function as RNA and DNA antisense sequences for the mutant genes ABCB1, ABCB4, ADRA1A, ADRA1D, ADRA2A, ADRB1, ADRB2, COMT, CRH, CRHBP, CRHR1, CRHR2, CYP2D6, CYP3A4, CYP2D19, DRD1, DRD2, DRD3, DRD4, GLRF1, HTR1A, HTR1B, HTR1D, HTR2A, HTR2B, HTR2C, HTR3A, HTR3B, MAOA, MAOB, NR3C1, SLC6A2 SLC6A3, SLC6A4, TAC1, TACR1 or TPH.

As can be seen, permutations can be generated for a selected mutant antigene by truncating the 5′ end, truncating the 3′ end, extending the 5′ end, or extending the 3′ end. Both light chain and heavy chain mtDNA can be targeted. Other variations such as truncating the 5′ end and truncating the 3′ end, extending the 5′ end and extending the 3′ end, and truncating the 5′ end and extending the 3′ end, extending the 5′ end and truncating the 3′ end, and so forth are possible.

The composition of the antisense or triplex oligonucleotides can also influence the efficiency of inhibition. For example, it is preferable to use oligonucleotides that are resistant to degradation by the action of endogenous nucleases. Nuclease resistance will confer a longer in vivo half-life to the oligonucleotide thus increasing its efficacy and reducing the required dose. Greater efficacy may also be obtained by modifying the oligonucleotide so that it is more permeable to cell membranes. Such modifications are well known in the art and include the alteration of the negatively charged phosphate backbone bases, or modification of the sequences at the 5′ or 3′ terminus with agents such as intercalators and crosslinking molecules. Specific examples of such modifications include oligonucleotide analogs that contain methylphosphonate (Miller, P. S., Biotechnology, 2:358-362 (1991)), phosphorothioate (Stein, Science 261:1004-1011 (1993)) and phosphorodithioate linkages (Brill, W. K-D., J. Am. Chem. Soc., 111:2322 (1989)). Other types of linkages and modifications exist as well, such as a polyamide backbone in peptide nucleic acids (Nielson et al., Science 254:1497 (1991)), formacetal (Matteucci, M., Tetrahedron Lett. 31:2385-2388 (1990)) carbamate and morpholine linkages as well as others known to those skilled in the art. In addition to the specificity afforded by the antisense agents, the target RNA or genes can be irreversibly modified by incorporating reactive functional groups in these molecules which covalently link the target sequences e.g. by alkylation.

Recombinant methods known in the art can also be used to achieve the antisense or triplex inhibition of a target nucleic acid. For example, vectors containing antisense nucleic acids can be employed to express protein or antisense message to reduce the expression of the target nucleic acid and therefore its activity. Such vectors are known or can be constructed by those skilled in the art and should contain all expression elements necessary to achieve the desired transcription of the antisense or triplex sequences. Other beneficial characteristics can also be contained within the vectors such as mechanisms for recovery of the nucleic acids in a different form. Phagemids are a specific example of such beneficial vectors because they can be used either as plasmids or as bacteriophage vectors. Examples of other vectors include viruses, such as bacteriophages, baculoviruses and retroviruses, cosmids, plasmids, liposomes and other recombination vectors. The vectors can also contain elements for use in either procaryotic or eukaryotic host systems. One of ordinary skill in the art will know which host systems are compatible with a particular vector.

The vectors can be introduced into cells or tissues by any one of a variety of known methods within the art. Such methods are described for example in Sambrook et al., Molecular Cloning: A Laboratory Manual, Cold Spring Harbor Laboratory, New York (1992), which is hereby incorporated by reference, and in Ausubel et al., Current Protocols in Molecular Biology, John Wiley and Sons, Baltimore, Md. (1989), which is also hereby incorporated by reference. The methods include, for example, stable or transient transfection, lipofection, electroporation and infection with recombinant viral vectors. Introduction of nucleic acids by infection offers several advantages over the other listed methods which includes their use in both in vitro and in vivo settings. Higher efficiency can also be obtained due to their infectious nature. Moreover, viruses are very specialized and typically infect and propagate in specific cell types. Thus, their natural specificity can be used to target the antisense vectors to specific cell types in vivo or within a tissue or mixed culture of cells. Viral vectors can also be modified with specific receptors or ligands to alter target specificity through receptor mediated events.

A specific example of a viral vector for introducing and expressing antisense nucleic acids is the adenovirus derived vector Adenop53TX. This vector expresses a herpes virus thymidine kinase (TX) gene for either positive or negative selection and an expression cassette for desired recombinant sequences such as antisense sequences. This vector can be used to infect cells including most cancers of epithelial origin, glial cells and other cell types. This vector as well as others that exhibit similar desired functions can be used to treat a mixed population of cells to selectively express the antisense sequence of interest. A mixed population of cells can include, for example, in vitro or ex vivo culture of cells, a tissue or a human subject.

Additional features may be added to the vector to ensure its safety and/or enhance its therapeutic efficacy. Such features include, for example, markers that can be used to negatively select against cells infected with the recombinant virus. An example of such a negative selection marker is the TK gene described above that confers sensitivity to the antibiotic gancyclovir. Negative selection is therefor a means by which infection can be controlled because it provides inducible suicide through the addition of antibiotics. Such protection ensures that if, for example, mutations arise that produce mutant forms of the viral vector or antisense sequence, cellular transformation will not occur. Moreover, features that limit expression to particular cell types can also be included. Such features include, for example, promoter and expression elements that are specific for the desired cell type.

The foregoing and following description of the invention and the various embodiments is not intended to be limiting of the invention but rather is illustrative thereof. Those skilled in the art of molecular genetics can formulate further embodiments encompassed within the scope of the present invention.

### FURTHER EXAMPLE OF TECHNIQUES

Definitions of Abbreviations:

1.times. SSC=150 mM sodium chloride, 15 mM sodium citrate, pH 6.5-8

SDS=sodium dodecyl sulfate

BSA=bovine serum albumin, fraction IV

probe=a labelled nucleic acid, generally a single-stranded oligonucleotide, which is complementary to the DNA target immobilized on the membrane. The probe may be labelled with radioisotopes (such as.sup.32P), haptens (such as digoxigenin), biotin, enzymes (such as alkaline phosphatase or horseradish peroxidase), fluorophores (such as fluorescein or Texas Red), or chemilumiphores (such as acridine).

PCR=polymerase chain reaction, as described by Erlich et al., Nature 331:461462 (1988) hereby incorporated by reference.

### Example III

Sequencing of ABCB1, ABCB4, ADRA1A, ADRA1D, ADRA2A, ADRB1, ADRB2, COMT, CRH, CRHBP, CRHR1, CRHR2, CYP2D6, CYP3A4, CYP2D19, DRD1, DRD2, DRD3, DRD4, GLRF1, HTR1A, HTR1B, HTR1D, and/HTR2A, HTR2B, HTR2C, HTR3A, HTR3B, MAOA, MAOB, NR3C1, SLC6A2 SLC6A3, SLC6A4, TAC1, TACR1 or TPH Genes

Plasmid DNA containing the ABCB1, ABCB4, ADRA1A, ADRA1D, ADRA2A, ADRB1, ADRB2, COMT, CRH, CRHBP, CRHR1, CRHR2, CYP2D6, CYP3A4, CYP2D19, DRD1, DRD2, DRD3, DRD4, GLRF1, HTR1A, HTR1B, HTR1D, HTR2A, HTR2B, HTR2C, HTR3A, HTR3B, MAOA, MAOB, NR3C1, SLC6A2 SLC6A3, SLC6A4, TAC1, TACR1 or TPH gene inserts is obtained as described in Example I is isolated using the Plasmid Quik.™. Plasmid Purification Kit (Stratagene, San Diego, Calif.) or the Plasmid Kit (Qiagen, Chatsworth, Calif., Catalog #12145). Plasmid DNA is purified from 50 ml bacterial cultures. For the Stratagene protocol “Procedure for Midi Columns,” steps 10-12 of the kit protocol are replaced with a precipitation step using 2 volumes of 100% ethanol at −20.degree. C., centrifugation at 6,000.times. g for 15 minutes, a wash step using 80% ethanol and resuspension of the DNA sample in 100 μl TE buffer. DNA concentration is determined by horizontal agarose gel electrophoresis, or by UV absorption at 260 nm.

Sequencing reactions using double-stranded plasmid DNA are performed using the Sequenase Kit (United States Biochemical Corp., Cleveland, Ohio.; catalog #70770), the BaseStation T7 Kit (Millipore Corp.; catalog #MBBLSEQ01), the Vent Sequencing Kit (Millipore Corp; catalog #MBBLVEN01), the AmpliTaq Cycle Sequencing Kit (Perkin Elmer Corp.; catalog #N808-0110) and the Taq DNA Sequencing Kit (Boehringer Mannheim). The DNA sequences are detected by fluorescence using the BaseStation Automated DNA Sequencer (Millipore Corp.). For gene walking experiments, fluorescent oligonucleotide primers are synthesized on the Cyclone Plus DNA Synthesizer (Millipore Corp.) or the GeneAssembler DNA Synthesizer (Pharmacia LKB Biotechnology, Inc.) utilizing beta-cyanoethylphosphoramidite chemistry. Primer sequences are prepared from the published Cambridge sequences of the ABCB1, ABCB4, ADRA1A, ADRA1D, ADRA2A, ADRB1, ADRB2, COMT, CRH, CRHBP, CRHR1, CRHR2, CYP2D6, CYP3A4, CYP2D19, DRD1, DRD2, DRD3, DRD4, GLRF1, HTR1A, HTR1B, HTR1D, HTR2A, HTR2B, HTR2C, HTR3A, HTR3B, MAOA, MAOB, NR3C1, SLC6A2 SLC6A3, SLC6A4, TAC1, TACR1 or TPH genes by using public reference sources such as http://www.snpperchip.org Primers are deprotected and purified as described above. DNA concentration is determined by UV absorption at 260 nm.

Sequencing reactions are performed according to manufacturer's instructions except for the following modification: 1) the reactions are terminated and reduced in volume by heating the samples without capping to 94.degree. C. for 5 minutes, after which 4 .mu.l of stop dye (3 mg/ml dextran blue, 95%-99% formamide; as formulated by Millipore Corp.) are added; 2) the temperature cycles performed for the AmpliTaq Cycle Sequencing Kit reactions, the Vent Sequencing kit reactions, and the Taq Sequence Kit consist of one cycle at 95.degree. C. for 10 seconds, 30 cycles at 95.degree. C. for 20 seconds, at 44.degree. C. for 20 seconds and at 72.degree. C. for 20 seconds followed by a reduction in volume by heating without capping to 94.degree. C. for 5 minutes before adding 4.mu.l of stop dye.

Electrophoresis and gel analysis are performed using the Biolmage and BaseStation Software provided by the manufacturer for the BaseStation Automated DNA Sequencer (Millipore Corp.). Sequencing gels are prepared according to the manufacturer's specifications. An average of ten different clones from each individual is sequenced. The resulting ABCB1, ABCB4, ADRA1A, ADRA1D, ADRA2A, ADRB1, ADRB2, COMT, CRH, CRHBP, CRHR1, CRHR2, CYP2D6, CYP3A4, CYP2D19, DRD1, DRD2, DRD3, DRD4, GLRF1, HTR1A, HTR1 B, HTR1 D, HTR2A, HTR2B, HTR2C, HTR3A, HTR3B, MAOA, MAOB, NR3C1, SLC6A2 SLC6A3, SLC6A4, TAC1, TACR1 or TPH sequences are aligned and compared with published Cambridge sequences. Mutations in the derived sequence are noted and confirmed by resequencing the variant region.

As an alternative procedure for sequencing the ABCB1, ABCB4, ADRA1A, ADRA1D, ADRA2A, ADRB1, ADRB2, COMT, CRH, CRHBP, CRHR1, CRHR2, CYP2D6, CYP3A4, CYP2D19, DRD1, DRD2, DRD3, DRD4, GLRF1, HTR1A, HTR1B, HTR1D, HTR2A, HTR2B, HTR2C, HTR3A, HTR3B, MAOA, MAOB, NR3C1, SLC6A2 SLC6A3, SLC6A4, TAC1, TACR1 or TPH genes, plasmid DNA containing the ABCB1, ABCB4, ADRA1A, ADRA1D, ADRA2A, ADRB1, ADRB2, COMT, CRH, CRHBP, CRHR1, CRHR2, CYP2D6, CYP3A4, CYP2D19, DRD1, DRD2, DRD3, DRD4, GLRF1, HTR1A, HTR1B, HTR1D, HTR2A, HTR2B, HTR2C, HTR3A, HTR3B, MAOA, MAOB, NR3C1, SLC6A2 SLC6A3, SLC6A4, TAC1, TACR1 or TPH gene inserts obtained as described in Example I is isolated using the Plasmid Quik.™. Plasmid Purification Kit with Midi Columns (Qiagen, Chatsworth, Calif.) Plasmid DNA is purified from 35 ml bacterial cultures. The isolated DNA is resuspended in 100 .mu.l TE buffer. DNA concentrations are determined by OD (260) absorption.

As an alternative method, sequencing reactions using double stranded plasmid DNA are performed using the Prism.™. Ready Reaction DyeDeoxy.™. Terminator Cycle Sequencing Kit (Applied Biosystems, Inc., Foster City, Calif.). The DNA sequences are detected by fluorescence using the ABI 373A Automated DNA Sequencer (Applied Biosystems, Inc., Foster City, Calif.). For gene walking experiments, oligonucleotide primers are synthesized on the ABI 394 DNA/RNA Synthesizer (Applied Biosystems, Inc., Foster City, Calif.) using standard beta-cyanoethylphosphoramidite chemistry. Primer sequences are prepared from the published Cambridge sequences of the ABCB1, ABCB4, ADRA1A, ADRA1D, ADRA2A, ADRB1, ADRB2, COMT, CRH, CRHBP, CRHR1, CRHR2, CYP2D6, CYP3A4, CYP2D19, DRD1, DRD2, DRD3, DRD4, GLRF1, HTR1A, HTR1B, HTR1D, and/HTR2A, HTR2B, HTR2C, HTR3A, HTR3B, MAOA, MAOB, NR3C1, SLC6A2 SLC6A3, SLC6A4, TAC1, TACR1 or TPH genes.

Sequencing reactions are performed according to the manufacturer's instructions. Electrophoresis and sequence analysis are performed using the ABI 373A Data Collection and Analysis Software and the Sequence Navigator Software (ABI, Foster City, Calif.). Sequencing gels are prepared according to the manufacturer's specifications. An average of ten different clones from each individual is sequenced. The resulting ABCB1, ABCB4, ADRA1A, ADRA1D, ADRA2A, ADRB1, ADRB2, COMT, CRH, CRHBP, CRHR1, CRHR2, CYP2D6, CYP3A4, CYP2D19, DRD1, DRD2, DRD3, DRD4, GLRF1, HTR1A, HTR1B, HTR1D, and/HTR2A, HTR2B, HTR2C, HTR3A, HTR3B, MAOA, MAOB, NR3C1, SLC6A2 SLC6A3, SLC6A4, TAC1, TACR1 or TPH sequences are aligned and compared with the published Cambridge sequence. Mutations in the derived sequence are noted and confirmed by sequence of the complementary DNA strand.

Mutations in each ABCB1, ABCB4, ADRA1A, ADRA1D, ADRA2A, ADRB1, ADRB2, COMT, CRH, CRHBP, CRHR1, CRHR2, CYP2D6, CYP3A4, CYP2D19, DRD1, DRD2, DRD3, DRD4, GLRF1, HTR1A, HTR1B, HTR1D, and/HTR2A, HTR2B, HTR2C, HTR3A, HTR3B, MAOA, MAOB, NR3C1, SLC6A2 SLC6A3, SLC6A4, TAC1, TACR1 or TPH gene(s) for each individual are compiled. Comparisons of mutations between normal and Depression patients are made and an algorithm, described below, is used to provide diagnostic or prognostic prediction.

### Example IV

Detection of ABCB1, ABCB4, ADRA1A, ADRA1D, ADRA2A, ADRB1, ADRB2, COMT, CRH, CRHBP, CRHR1, CRHR2, CYP2D6, CYP3A4, CYP2D19, DRD1, DRD2, DRD3, DRD4, GLRF1, HTR1A, HTR1B, HTR1D, and/HTR2A, HTR2B, HTR2C, HTR3A, HTR3B, MAOA, MAOB, NR3C1, SLC6A2 SLC6A3, SLC6A4, TAC1, TACR1 or TPH Mutations by Hybridization Without Prior Amplification

This example illustrates taking test sample blood, blotting the DNA, and detecting by oligonucleotide hybridization in a dot blot format. This example uses two probes to determine the presence of the abnormal mutations of the ABCB1, ABCB4, ADRA1A, ADRA1D, ADRA2A, ADRB1, ADRB2, COMT, CRH, CRHBP, CRHR1, CRHR2, CYP2D6, CYP3A4, CYP2D19, DRD1, DRD2, DRD3, DRD4, GLRF1, HTR1A, HTR1B, HTR1D, HTR2A, HTR2B, HTR2C, HTR3A, HTR3B, MAOA, MAOB, NR3C1, SLC6A2 SLC6A3, SLC6A4, TAC1, TACR1 or TPH gene(s) DNA of Depression patients. This example utilizes a dot-blot format for hybridization, however, other known hybridization formats, such as Southern blots, slot blots, “reverse” dot blots, solution hybridization, solid support based sandwich hybridization, bead-based, silicon chip-based and microtiter well-based hybridization formats can also be used.

Sample Preparation Extracts and Blotting of DNA onto Membranes:

Whole blood is taken from the patient. The blood is mixed with an equal volume of 0.5-1 N NaOH, and is incubated at ambient temperature for ten to twenty minutes to lyse cells, degrade proteins, and denature any DNA. The mixture is then blotted directly onto prewashed nylon membranes, in multiple aliquots. The membranes are rinsed in 10.times. SSC (1.5 M NaCl, 0.15 M Sodium Citrate, pH 7.0) for five minutes to neutralize the membrane, then rinsed for five minutes in 1.times. SSC. For storage, if any, membranes are air-dried and sealed. In preparation for hybridization, membranes are rinsed in 1.times. SSC, 1% SDS.

Alternatively, 1-10 mls of whole blood is fractionated by standard methods, and the white cell layer (“buffy coat”) is separated. The white cells are lysed, digested, and the DNA extracted by conventional methods (organic extraction, non-organic extraction, or solid phase). The DNA is quantitated by UV absorption or fluorescent dye techniques. Standardized amounts of DNA (0.1-5 .mu.g) are denatured in base, and blotted onto membranes. The membranes are then rinsed.

Alternative methods of preparing cellular DNA, such as isolation of DNA by mild cellular lysis and centrifugation, may also be used.

Hybridization and Detection:

For examples of synthesis, labelling, use, and detection of oligonucleotide probes, see “Oligonucleotides and Analogues: A Practical Approach”, F. Eckstein, ed., Oxford University Press (1992); and “Synthetic Chemistry of Oligonucleotides and Analogs”, S. Agrawal, ed., Humana Press (1993), which are incorporated herein by reference.

For detection and quantitation of the abnormal mutation, membranes containing duplicate samples of DNA are hybridized in parallel; one membrane is hybridized with the wild-type probe, the other with the Depression gene probe. Alternatively, the same membrane can be hybridized sequentially with both probes and the results compared.

For example, the membranes with immobilized DNA are hydrated briefly (10-60 minutes) in 1.times. SSC, 1% SDS, then prehybridized and blocked in 5.times. SSC, 1% SDS, 0.5% casein, for 30-60 minutes at hybridization temperature (35-60.degree. C., depending on which probe is used). Fresh hybridization solution containing probe (0.1-10 nM, ideally 2-3 nM) is added to the membrane, followed by hybridization at appropriate temperature for 15-60 minutes. The membrane is washed in 1.times. SSC, 11 SDS, 1-3 times at 45-60.degree. C. for 5-10 minutes each (depending on probe used), then 1-2 times in 1.times. SSC at ambient temperature. The hybridized probe is then detected by appropriate means.

The average proportion of Depression ABCB1, ABCB4, ADRA1A, ADRA1D, ADRA2A, ADRB1, ADRB2, COMT, CRH, CRHBP, CRHR1, CRHR2, CYP2D6, CYP3A4, CYP2D19, DRD1, DRD2, DRD3, DRD4, GLRF1, HTR1A, HTR1B, HTR1D, HTR2A, HTR2B, HTR2C, HTR3A, HTR3B, MAOA, MAOB, NR3C1, SLC6A2 SLC6A3, SLC6A4, TAC1, TACR1 or TPH gene(s) to wild-type gene(s) in the same patient can be determined by the ratio of the signal of the Depression probe to the normal probe. This is a semiquantitative measure of % heteroplasmy in the Depression patient and can be correlated to the severity of the disease.

The above and other probes for alteration and quantitation of wild-type and mutant DNA samples can be found at http://www.snpper.chip.org and typing in the RS numbers of the relevant mutations.

### Example V

Detection of ABCB1, ABCB4, ADRA1A, ADRA1D, ADRA2A, ADRB1, ADRB2, COMT, CRH, CRHBP, CRHR1, CRHR2, CYP2D6, CYP3A4, CYP2D19, DRD1, DRD2, DRD3, DRD4, GLRF1, HTR1A, HTR1B, HTR1D, HTR2A, HTR2B, HTR2C, HTR3A, HTR3B, MAOA, MAOB, NR3C1, SLC6A2 SLC6A3, SLC6A4, TAC1, TACR1 OR TPH Mutations by Hybridization (Without Prior Amplification)

A. Slot-Blot Detection of RNA/DNA with .sup.32P Probes

This example illustrates detection of ABCB1, ABCB4, ADRA1A, ADRA1D, ADRA2A, ADRB1, ADRB2, COMT, CRH, CRHBP, CRHR1, CRHR2, CYP2D6, CYP3A4, CYP2D19, DRD1, DRD2, DRD3, DRD4, GLRF1, HTR1A, HTR1B, HTR1D, HTR2A, HTR2B, HTR2C, HTR3A, HTR3B, MAOA, MAOB, NR3C1, SLC6A2 SLC6A3, SLC6A4, TAC1, TACR1 or TPH mutations by slot-blot detection of DNA with .sup.32p probes. The reagents are prepared as follows: 4.times. BP: 2% (w/v) Bovine serum albumin (BSA), 2% (w/v) polyvinylpyrrolidone (PVP, Mol. Wt.: 40,000) is dissolved in sterile H.sub.2O and filtered through 0.22-.mu. cellulose acetate membranes (Coming) and stored at −20.degree. C. in 50-ml conical tubes.

DNA is denatured by adding TE to the sample for a final volume of 90 .mu.l. 10 .mu.l of 2 N NaOH is then added and the sample vortexed, incubated at 65.degree. C. for 30 minutes, and then put on ice. The sample is neutralized with 100 .mu.l of 2 M ammonium acetate.

A wet piece of nitrocellulose or nylon is cut to fit the slot-blot apparatus according to the manufacturer's directions, and the denatured samples are loaded. The nucleic acids are fixed to the filter by baking at 80.degree. C. under vacuum for 1 hr or exposing to UV light (254 nm). The filter is prehybridized for 10-30 minutes in 5 mls of 1.times. BP, 5.times. SSPE, 1% SDS at the temperature to be used for the hybridization incubation. For 15-30-base probes, the range of hybridization temperatures is between 35-60.degree. C. For shorter probes or probes with low G-C content, a lower temperature is used. At least 2.times.10.sup.6 cpm of detection oligonucleotide per ml of hybridization solution is added. The filter is double sealed in Scotchpak.™. heat sealable pouches (Kapak Corporation) and incubated for 90 min. The filter is washed 3 times at room temperature with 5-minute washes of 20.times. SSPE: 3M NaCl, 0.02M EDTA, 0.2 Sodium Phospate, pH 7.4, 1% SDS on a platform shaker. For higher stringency, the filter can be washed once at the hybridization temperature in 1.times. SSPE, 1% SDS for 1 minute. Visualization is by autoradiography on Kodak XAR film at −70.degree. C. with an intensifying screen. To estimate the amount of target, compare the amount of target detected by visual comparison with hybridization standards of known concentration.

B. Detection of RNA/DNA by Slot-Blot Analysis with Alkaline Phosphatase-Oligonucleotide Conjugate Probes

This example illustrates detection of ABCB1, ABCB4, ADRA1A, ADRA1D, ADRA2A, ADRB1, ADRB2, COMT, CRH, CRHBP, CRHR1, CRHR2, CYP2D6, CYP3A4, CYP2D19, DRD1, DRD2, DRD3, DRD4, GLRF1, HTR1A, HTR1B, HTR1D, HTR2A, HTR2B, HTR2C, HTR3A, HTR3B, MAOA, MAOB, NR3C1, SLC6A2 SLC6A3, SLC6A4, TAC1, TACR1 or TPH mutations by slot-blot detection of DNA with alkaline phosphatase-oligonucleotide conjugate probes, using either a color reagent or a chemiluminescent reagent. The reagents are prepared as follows:

Color Reagent:

For the color reagent, the following are mixed together, fresh 0.16 mg/ml 5-bromo4-chloro-3-indolyl phosphate (BCIP), 0.17 mg/ml nitroblue tetrazolium (NBT) in 100 mM NaCl, 100 mM Tris. HCl, 5 mM MgCl.sub.2 and 0.1 mM ZnCl.sub.2, pH 9.5.

Chemiluminescent Reagent:

For the chemiluminescent reagent, the following are mixed together, 250 .mu.M 3-adamantyl 4-methoxy 4-(2-phospho)phenyl dioxetane (AMPPD), (Tropix Inc., Bedford, Mass.) in 100 mM diethanolamine-HCl, 1 mM MgCl.sub.2 pH 9.5, or preformulated dioxetane substrate Lumiphos.™. 530 (Lumigen, Inc., Southfield, Mich.).

DNA target (0.01-50 fmol) is immobilized on a nylon membrane as described above. The nylon membrane is incubated in blocking buffer (0.2% I-Block (Tropix, Inc.), 0.5.times. SSC, 0.1% Tween 20) for 30 min. at room temperature with shaking. The filter is then prehybridized in hybridization solution (5.times. SSC, 0.5% BSA, 1% SDS) for 30 minutes at the hybridization temperature (37-60.degree. C.) in a sealable bag using 50-100 .mu.l of hybridization solution per cm of membrane. The solution is removed and briefly washed in warm hybridization buffer. The conjugate probe is then added to give a final concentration of 2-5 nM in fresh hybridization solution and final volume of 50-100 .mu.l/cm.sup.2 of membrane. After incubating for 30 minutes at the hybridization temperature with agitation, the membrane is transferred to a wash tray containing 1.5 ml of preheated wash-1 solution (1.times. SSC, 0.1% SDS)/cm.sup.2 of membrane and agitated at the wash temperature (usually optimum hybridization temperature minus 10.degree. C.) for 10 minutes. Wash-1 solution is removed and this step is repeated once more. Then wash-2 solution (1.times. SSC) added and then agitated at the wash temperature for 10 minutes. Wash-2 solution is removed and immediate detection is done by color.

Detection by color is done by immersing the membrane fully in color reagent, and incubating at 20-37.degree. C. until color development is adequate. When color development is adequate, the development is quenched by washing in water.

For chemiluminescent detection, the following wash steps are performed after the hybridization step (see above). Thus, the membrane is washed for 10 min. with wash-i solution at room temperature, followed by two 3-5 min. washes at 50-60.degree. C. with wash-3 solution (0.5′ SSC, 0.1% SDS). The membrane is then washed once with wash-4 solution (1.times. SSC, 1% Triton X 100) at room temperature for 10 min., followed by a 10 min. wash at room temperature with wash-2 solution. The membrane is then rinsed briefly (.about.1 min.) with wash-5 solution (50 mM NaHCO.sub.3/1 mM MgCl.sub.2, pH 9.5).

Detection by chemiluminescence is done by immersing the membrane in luminescent reagent, using 25-5 .mu.l solution/cm.sup.2 of membrane. Kodak XAR-5 film (or equivalent; emission maximum is at 477 .mu.m) is exposed in a light-tight cassette for 1-24 hours, and the film developed.

### Example VI

Detection of ABCB1, ABCB4, ADRA1A, ADRA1D, ADRA2A, ADRB1, ADRB2, COMT, CRH, CRHBP, CRHR1, CRHR2, CYP2D6, CYP3A4, CYP2D19, DRD1, DRD2, DRD3, DRD4, GLRF1, HTR1A, HTR1B, HTR1D, HTR2A, HTR2B, HTR2C, HTR3A, HTR3B, MAOA, MAOB, NR3C1, SLC6A2 SLC6A3, SLC6A4, TAC1, TACR1 or TPH Mutations by Amplification and Hybridization

This example illustrates taking a test sample of blood, preparing DNA, amplifying a section of a specific ABCB1, ABCB4, ADRA1A, ADRA1D, ADRA2A, ADRB1, ADRB2, COMT, CRH, CRHBP, CRHR1, CRHR2, CYP2D6, CYP3A4, CYP2D19, DRD1, DRD2, DRD3, DRD4, GLRF1, HTR1A, HTR1B, HTR1D, and/HTR2A, HTR2B, HTR2C, HTR3A, HTR3B, MAOA, MAOB, NR3C1, SLC6A2 SLC6A3, SLC6A4, TAC1, TACR1 or TPH gene(s) by polymerase chain reaction (PCR), and detecting the mutation by oligonucleotide hybridization in a dot blot format.

Sample Preparation and Preparing of DNA:

Whole blood is taken from the patient. The blood is lysed, and the DNA prepared for PCR by using procedures described in Example 1.

Amplification of Target ABCB1, ABCB4, ADRA1A, ADRA1D, ADRA2A, ADRB1, ADRB2, COMT, CRH, CRHBP, CRHR1, CRHR2, CYP2D6, CYP3A4, CYP2D19, DRD1, DRD2, DRD3, DRD4, GLRF1, HTR1A, HTR1B, HTR1D, and/HTR2A, HTR2B, HTR2C, HTR3A, HTR3B, MAOA, MAOB, NR3C1, SLC6A2 SLC6A3, SLC6A4, TAC1, TACR1 or TPH Gene(s) by Polymerase Chain Reaction, and Blotting onto Membranes:

The treated DNA from the test sample is amplified using procedures described in Example 1. After amplification, the DNA is denatured, and blotted directly onto prewashed nylon membranes, in multiple aliquots. The membranes are rinsed in 10.times. SSC for five minutes to neutralize the membrane, then rinsed for five minutes in 1.times. SSC. For storage, if any, membranes are air-dried and sealed. In preparation for hybridization, membranes are rinsed in 1.times. SSC, 1% SDS.

Hybridization and Detection:

Hybridization and detection of the amplified genes are accomplished as detailed in Example III.

Although the invention has been described with reference to the disclosed embodiments, those skilled in the art will readily appreciate that the specific examples provided herein are only illustrative of the invention and not limitative thereof. It should be understood that various modifications can be made without departing from the scope of the invention.

### Example VII

Synthesis of Antisense Oligonucleotides

Standard manufacturer protocols for solid phase phosphoramidite-based DNA or RNA synthesis using an ABI DNA synthesizer are employed to prepare antisense oligomers. Phosphoroamidite reagent monomers (T, C, A, G, and U) are used as received from the supplier. Applied Biosystems Division/Perkin Elmer, Foster City, Calif. For routine oligomer synthesis, 1 .mu.mole scale syntheses reactions are carried out utilizing THF/I.sub.2/lutidine for oxidation of the phosphoramidite and Beaucage reagent for preparation of the phosphorothioate oligomers. Cleavage from the solid support and deprotection are carried out using ammonium hydroxide under standard conditions. Purification is carried out via reverse phase HPLC and quantification and identification is performed by UV absorption measurements at 260 nm, and mass spectrometry.

### Example VIII

Inhibition of Mutant DNA in Cell Culture

Antisense phosphorothioate oligomer complementary to the ABCB1, ABCB4, ADRA1A, ADRA1D, ADRA2A, ADRB1, ADRB2, COMT, CRH, CRHBP, CRHR1, CRHR2, CYP2D6, CYP3A4, CYP2D19, DRD1, DRD2, DRD3, DRD4, GLRF1, HTR1A, HTR1B, HTR1D, and/HTR2A, HTR2B, HTR2C, HTR3A, HTR3B, MAOA, MAOB, NR3C1, SLC6A2 SLC6A3, SLC6A4, TAC1, TACR1 or TPH gene mutation(s) and thus non-complementary to wild-type ABCB1, ABCB4, ADRA1A, ADRA1D, ADRA2A, ADRB1, ADRB2, COMT, CRH, CRHBP, CRHR1, CRHR2, CYP2D6, CYP3A4, CYP2D19, DRD1, DRD2, DRD3, DRD4, GLRF1, HTR1A, HTR1B, HTR1D, and/or SLC6A gene mutant RNA(s), respectively, is added to fresh medium containing-Lipofectin.R™. Gibco BRL (Gaithersburg, Md.) at a concentration of 10.mu.g/ml to make final concentrations of 0.1, 0.33, 1, 3.3, and 10.mu.M. These are incubated for 15 minutes then applied to the cell culture. The culture is allowed to incubate for 24 hours and the cells are harvested and the DNA isolated and sequenced as in previous examples. Quantitative analysis results shows a decrease in mutant ABCB1, ABCB4, ADRA1A, ADRA1D, ADRA2A, ADRB1, ADRB2, COMT, CRH, CRHBP, CRHR1, CRHR2, CYP2D6, CYP3A4, CYP2D19, DRD1, DRD2, DRD3, DRD4, GLRF1, HTR1A, HTR1B, HTR1D, HTR2A, HTR2B, HTR2C, HTR3A, HTR3B, MAOA, MAOB, NR3C1, SLC6A2 SLC6A3, SLC6A4, TAC1, TACR1 or TPH DNA(s) to a level of less than 1% of total ABCB1, ABCB4, ADRA1A, ADRA1D, ADRA2A, ADRB1, ADRB2, COMT, CRH, CRHBP, CRHR1, CRHR2, CYP2D6, CYP3A4, CYP2D19, DRD1, DRD2, DRD3, DRD4, GLRF1, HTR1A, HTR1B, HTR1D, and/HTR2A, HTR2B, HTR2C, HTR3A, HTR3B, MAOA, MAOB, NR3C1, SLC6A2 SLC6A3, SLC6A4, TAC1, TACR1 or TPH, respectively.

The antisense phosphorothioate oligomer non-complementary to the ABCB1, ABCB4, ADRA1A, ADRA1D, ADRA2A, ADRB1, ADRB2, COMT, CRH, CRHBP, CRHR1, CRHR2, CYP2D6, CYP3A4, CYP2D19, DRD1, DRD2, DRD3, DRD4, GLRF1, HTR1A, HTR1B, HTR1D, and/HTR2A, HTR2B, HTR2C, HTR3A, HTR3B, MAOA, MAOB, NR3C1, SLC6A2 SLC6A3, SLC6A4, TAC1, TACR1 or TPH gene mutation(s) and non-complementary to wild-type ABCB1, ABCB4, ADRA1A, ADRA1D, ADRA2A, ADRB1, ADRB2, COMT, CRH, CRHBP, CRHR1, CRHR2, CYP2D6, CYP3A4, CYP2D19, DRD1, DRD2, DRD3, DRD4, GLRF1, HTR1A, HTR1B, HTR1D, and/HTR2A, HTR2B, HTR2C, HTR3A, HTR3B, MAOA, MAOB, NR3C1, SLC6A2 SLC6A3, SLC6A4, TAC1, TACR1 or TPH, respectively is added to fresh medium containing lipofectin at a concentration of 10 .mu.g/mL to make final concentrations of 0. 1, 0.33, 1, 3.3, and 10.mu.M. These are incubated for 15 minutes then applied to the cell culture. The culture is allowed to incubate for 24 hours and the cells are harvested and the DNA isolated and sequenced as in previous examples. Quantitative analysis results showed no decrease in mutant ABCB1, ABCB4, ADRA1A, ADRA1D, ADRA2A, ADRB1, ADRB2, COMT, CRH, CRHBP, CRHR1, CRHR2, CYP2D6, CYP3A4, CYP2D19, DRD1, DRD2, DRD3, DRD4, GLRF1, HTR1A, HTR1B, HTR1D, and/HTR2A, HTR2B, HTR2C, HTR3A, HTR3B, MAOA, MAOB, NR3C1, SLC6A2 SLC6A3, SLC6A4, TAC1, TACR1 or TPH DNA, respectively.

### Example IX

Inhibition of Mutant DNA In Vivo

Mice are divided into six groups of 10 animals per group. The animals are housed and fed as per standard protocols. To groups 1 to 4 is administered ICV, antisense phosphorothioate oligonucleotide, prepared as described in Example V, complementary to mutant ABCB1, ABCB4, ADRA1A, ADRA1D, ADRA2A, ADRB1, ADRB2, COMT, CRH, CRHBP, CRHR1, CRHR2, CYP2D6, CYP3A4, CYP2D19, DRD1, DRD2, DRD3, DRD4, GLRF1, HTR1A, HTR1B, HTR1D, and/HTR2A, HTR2B, HTR2C, HTR3A, HTR3B, MAOA, MAOB, NR3C1, SLC6A2 SLC6A3, SLC6A4, TAC1, TACR1 or TPH gene RNA(s), respectively 0.1, 0.33, 1.0 and 3.3 nmol each in 5.mu.L. To group 5 is administered ICV 1.0 nmol in 5.mu.L of phosphorothioate oligonucleotide non-complementary to mutant ABCB1, ABCB4, ADRA1A, ADRA1D, ADRA2A, ADRB1, ADRB2, COMT, CRH, CRHBP, CRHR1, CRHR2, CYP2D6, CYP3A4, CYP2D19, DRD1, DRD2, DRD3, DRD4, GLRF1, HTR1A, HTR1B, HTR1D, and/HTR2A, HTR2B, HTR2C, HTR3A, HTR3B, MAOA, MAOB, NR3C1, SLC6A2 SLC6A3, SLC6A4, TAC1, TACR1 or TPH gene RNA(s) and non-complementary to wild-type ABCB1, ABCB4, ADRA1A, ADRA1D, ADRA2A, ADRB1, ADRB2, COMT, CRH, CRHBP, CRHR1, CRHR2, CYP2D6, CYP3A4, CYP2D19, DRD1, DRD2, DRD3, DRD4, GLRF1, HTR1A, HTR1B, HTR1D, and/HTR2A, HTR2B, HTR2C, HTR3A, HTR3B, MAOA, MAOB, NR3C1, SLC6A2 SLC6A3, SLC6A4, TAC1, TACR1 or TPH gene RNA(s), respectively. To group 6 is administered ICV vehicle only. Dosing is performed once a day for ten days. The animals are sacrificed and samples of relevant tissue collected. This tissue is treated as previously described and the DNA isolated and quantitatively analyzed as in previous examples. Results show a decrease in mutant ABCB1, ABCB4, ADRA1A, ADRA1D, ADRA2A, ADRB1, ADRB2, COMT, CRH, CRHBP, CRHR1, CRHR2, CYP2D6, CYP3A4, CYP2D19, DRD1, DRD2, DRD3, DRD4, GLRF1, HTR1A, HTR1B, HTR1D, and/or HTR2A, HTR2B, HTR2C, HTR3A, HTR3B, MAOA, MAOB, NR3C1, SLC6A2 SLC6A3, SLC6A4, TAC1, TACR1 or TPH DNA to a level of less than 1% of total ABCB1, ABCB4, ADRA1A, ADRA1D, ADRA2A, ADRB1, ADRB2, COMT, CRH, CRHBP, CRHR1, CRHR2, CYP2D6, CYP3A4, CYP2D19, DRD1, DRD2, DRD3, DRD4, GLRF1, HTR1A, HTR1B, HTR1D, and/HTR2A, HTR2B, HTR2C, HTR3A, HTR3B, MAOA, MAOB, NR3C1, SLC6A2 SLC6A3, SLC6A4, TAC1, TACR1 or TPH for the antisense treated group and no decrease for the control group.

Algorithmic methodology for determining relevance of combinations of mutations to depression diagnosis or prognosis

As was mentioned previously, combinations of mutations might combine in non-linear fashion in determining their effect on diagnosis and prognosis. The present invention demonstrates this as well. A previous example showed that using a trained learning algorithm of neural network and support vector machine type, an average predictability rate of 84% could be achieved in a population that the trained algorithm had never seen before, i.e. an evaluative population.

It is well known to those of ordinary skill in the art that predictive algorithms have three measures of testing, each of increasing validation: how well the algorithm does on data it has learned, called a training population; how well the algorithm does on data that is similar to the original dataset but not trained on, called a testing population; and how well the algorithm does on data it has never seen before, called an evaluation population. What is extremely spectacular about the present invention is its level of predictability in an evaluation population, which indicates its generalizability to a larger population.

It is therefore important to realize that in order to be interpreted into a clinical result that an algorithm must be used to determine the individual contribution each marker makes to the phenotype of interest.

As with identification of the pertinent alleles in the first instance, a algorithm is both (i) selected and (ii) trained to relate (i) identified pre-selected markers and/or characteristics of SNP patterns (as selectively appear in the genomic sequences of each of large number of historical patients) with (ii) the clinical histories of the response of these patients to some particular disease (e.g., breast cancer) in consideration of therapies applied, most commonly drugs. As before, (i) selecting and (ii) training the algorithm to the commonly vast historical clinical data, and to some scores or even hundreds of alleles, is a computationally intensive task normally performed over the period of some hours or days on a supercomputer.

Properly performed—and causal relationships, howsoever complex and permuted, residing somewhere within the data—the resulting (i) selected, and (ii) trained, algorithm will itself be the “synthesis solution”. The algorithm will itself be the expression of what can be known from the data. The later use, and exercise, of the algorithm is only so as to give “answers” for particular questions (i.e., what should be expected from administration of some particular drug) for particular patients (i.e., as are possessed of a particular pattern of markers and/or SNP pattern). Notably, the algorithm can exercised so as to validate its own performance (or lack thereof). The clinical data for the many patients, and patient histories, can be fed into the (selected, trained) algorithm, one patient at a time. Does the algorithm accurately predict what historical data shows to have actually happened? A properly selected and trained algorithm is normally much more accurate in its prognostications (for the useful questions that it may suitably answer) than is any human physician. The physician's judgment ultimately controls, but the “advice” of the algorithm “solution” constitutes a useful adjunct to the physician's judgment in the considerably complex area of relating a patient's therapy to his or her genetic profile.

Methodology of Marker Selection, Analysis, and Classification

Non-linear techniques for data analysis and information extraction are important for identifying complex interactions between markers that contribute to overall presentation of the clinical outcome. However, due to the many features involved in association studies such as the one proposed, the construction of these in-silico predictors is a complex process. Often one must consider more markers to test than samples, missing values, poor generalization of results, selection of free parameters in predictor models, confidence in finding a sub-optimal solution and others. Thus, the process for building a predictor is as important as designing the protocol for the association studies. Errors at each step can propagate downstream, affecting the generalizability of the final result.

We now provide an overview of our process of model development, describing the five main steps and some techniques that the instant invention will use to build an optimal biomarker panel of response for each clinical outcome. One of ordinary skill in the art will know that it is best to use a ‘toolbox’ approach to the various steps, trying several different algorithms at each step, and even combining several as in Step Five. Since one does not know a priori the distribution of the true solution space, trying several methods allows a thorough search of the solution space of the observed data in order to find the most optimal solutions (i.e. those best able to generalize to unseen data). One also can give more confidence to predictions if several independent techniques converge to a similar solution.

Data Pre-Processing

After assaying the patients for various markers, it is necessary to perform some basic data ‘inspection’, such as identification of outliers, before starting a program of outcome prediction. Another task is performing data dimensional shifting in the case of discrete data sets such as SNP analysis. For instance, one can describe a three-state SNP vector either three-dimensionally (1,0,0);(0,1,0);(0,0,1) or two-dimensionally (0,0);(1,0);(0,1). For some algorithms, the latter description may have a direct effect on computational cost and classifier accuracy: one can, in effect, collapse several values to a single parameter. The advantage of single parameter is that one can reduce dimensionality with little or no effect on the selection of the optimal feature set. Following pre-processing, one can then perform univariate and multivariate statistical modeling to identify strongly correlative outcome variables and determine a baseline outcome analysis.

Missing Value Estimation

While the call rate and accuracy of high throughput methods are improving, genotype and proteomic data sets usually contain missing values. Missing values arise from missed genotype calls or from the combination of data collected under different protocols. If subsequent analysis requires complete data sets, repeating the experiment can be expensive and removing rows or columns containing missing values in the data set may be wasteful.

Missing values can be replaced with the most likely genotype based on frequency estimates for an individual marker. This row counting method may be sufficient when few markers are genotyped, but it is not optimal for genome wide scans since it does not consider correlation in the data. Other statistical approaches to estimating missing values apply genetic models of inheritance. In large-scale association studies of unrelated participants, lineage information is unavailable. For the dataset gathered in the instant invention, we will apply techniques that do not use complex models and take into account the possibly discrete nature of marker data when models are used. These methods fall into two categories: KNN-based and Bayesian-based methods.

KNN estimates the value of the missing data as the most prevalent genotype among the K Nearest Neighbors. For a data set consisting of M patients and N SNPs, the data is stored in an M by N matrix. For each row with a missing value in a single column, the algorithm locates the K nearest neighbors in the N-1 dimensional subspace. The K nearest neighbors then votes to replace the missing value under majority rule. Ties are broken by random draw. If there are n missing values present in a row, we find the nearest neighbors in the N-n subspace.

The only other consideration is what distance function to use to determine the K nearest neighbors. Typically, the Euclidean distance is well suited for continuous data and the Hamming distance for nominal data. The Hamming distance counts the number of different marker genotypes in the N-n subspace and does not impose an artificial ordinality as does the Euclidean distance. There are other options such as the Manhattan distance, the correlation coefficient, and others that may be used depending on the data set distribution.

In contrast, Bayesian imputation uses probabilities instead of distances to infer missing values. The objective is to draw an inference about a missing value for a matrix entry in the data set from the posterior probability of the missing value given the observed data, □(Yₘᵢₛₛ|Y_{obs}), where Y_{obs} is the set of N-n observed marker values and Yₘᵢₛₛ is the missing value. By Bayes's theorem, □(Yₘᵢₛₛ|Y_{obs}) can be expressed as follows:

$$π ( {Y}_{miss} \vert {Y}_{obs} ) = \frac{π ( {Y}_{obs} \vert {Y}_{mis} ) π ( {Y}_{miss} )}{{\sum }_{k = 1}^{m} π ( {Y}_{obs} \vert {Y}_{mis} ) π ( {Y}_{miss} )} \qquad ( 11 )$$

where π(Yₘᵢₛₛ) is the probability that a randomly selected missing entry will have the value Yₘᵢₛₛ, π(Y_{obs}|Yₘᵢₛₛ) is the probability of observing the N-n genotypes given Yₘᵢₛₛ, and the sum is over the m possible values for Yₘᵢₛₛ.

The likelihood model assumes that the probabilities π(Y_{obs}|Yₘᵢₛₛ) can be expressed as functions of unknown parameters of the genotypes Yₘᵢₛₛ:

$$\begin{matrix}π ( {Y}_{obs = g} \vert {Y}_{miss = k} ) = π ( {y}_{g1} \vert {θ}_{1 k} ) π ( {y}_{g2} \vert {θ}_{2 k} ) … π ( {y}_{gn} \vert {θ}_{nk} ) \\ = {\prod }_{i = 1}^{N - n} π ( {y}_{gi} \vert {θ}_{ik} )\end{matrix} \qquad ( 2 )$$

where θᵢₖ are unknown parameters of Yₘᵢₛₛ for the N-n observed markers, y_{gi} is the i th marker in the set of Y_{obs} markers, and θ (y_{gi}|θᵢₖ) is the probability of observing y_{gi} given the parameter θᵢₖ of the marker value Yₘᵢₛₛ for variable i. The model is based on the assumption that the probability of observing y_{gi} is independent of the probability of observing y_{gi} for each marker value Yₘᵢₛₛ with i≠j.

Missing values are imputed as follows. For each marker for which there is a missing value, the probabilities θ (y_{gi}|θᵢₖ) are estimated based on the observed markers. Using Bayes' theorem, the posterior probability θ (Yₘᵢₛₛ|Y_{obs}) is calculated. We then sample Yₘᵢₛₛ from the posterior. This approach treats the missing value problem as a supervised learning problem in which posterior probability is learned from the pattern of observed markers.

Feature Selection

Following missing value replacement, the third step in the predictive panel building process is to perform feature selection on the dataset; this is perhaps the most important step in the predictor development process. Feature selection serves two purposes: (1) to reduce dimensionality of the data and improve classification accuracy, and (2) to identify biomarkers that are relevant to the cause and consequences of disease and drug response.

A feature selection algorithm (FSA) is a computational solution that given a set of candidate features selects a subset of relevant features with the best commitment among its size and the value of its evaluation measure. However, the relevance of a feature, as seen from the classification perspective, may have several definitions depending on the objective desired. An irrelevant feature is not useful for classification, but not all relevant features are necessarily useful for classification.

Another problem from which many classification methods suffer is the curse of dimensionality. That is, as the number of features in a classification task increases, the time requirements for an algorithm grow dramatically, sometimes exponentially. Therefore, when the set of features in the data is sufficiently large, many classification algorithms are simply intractable. This problem is further exacerbated by the fact that many features in a learning task may either be irrelevant or redundant to other features with respect to predicting the class of an instance. In this context, such features serve no purpose except to increase classification time.

FSAs can be divided into two categories based on whether or not feature selection is done independently of the learning algorithm used to construct the classifier. If the feature selection is independent of the learning algorithm, the technique is said to follow a filter approach. Otherwise, it is said to follow a wrapper approach. While the filter approach is generally computationally more efficient than the wrapper approach, a drawback is that an optimal selection of features may not be independent of the inductive and representational biases of the learning algorithm to be used to construct the classifier.

SFS/SBS

A sequential forward search (SFS), or backward (SBS), is a process that uses an iterative technique for feature selection. In this wrapper technique, one feature at a time is added (SFS) or deleted (SBS) to a set of pre-selected features, and iterated according to a performance metric until the ‘optimal’ set of features are obtained. For example, SFS is a technique that starts with all possible two-variable input combinations from the entire data set and then builds, one variable at a time, until an optimally performing combination of variables is identified. For instance, with 9 input variables labeled 1-9 (each with a binary descriptor), the two-variable combinations would comprise 1|2, 1|3, 1|4, 1|5, 1|6, 1|7, 1|8, 1|9, 2|3, 2|4, 2|5, 2|6 . . . 8|9. These input combinations are each used in training a classifier using the collected data. The combinations that perform the best (evaluated using leave-one-out cross validation; top 10%, for example) are selected for continued addition of variables. Let us say that 2|3 is selected as one of the top performers, it would then be coupled to each of the other variables, not including those variables that are already included in the combination. This would result in 2|3|1, 2|3|4, 2|3|5, 2|3|6, 2|3|7, 2|3|8 and 2|3|9. This coupling is performed for all of the top two-variable performers. The resultant three-variable input combinations are used to train a classifier using the collected data and then evaluated. The top performers are selected and then coupled again with all variables in the group, again used to train a classifier. This is repeated until a maximal predictive accuracy is achieved. In our experience we have noticed a well defined ‘hump’ at the point where the addition of variables into the system results begins to contribute to degradation of system performance.

SBS starts with the full set of features and eliminates those based upon a performance metric. Although in theory, going backward from the full set of features may capture interacting features more easily, the drawback of this method is that it is computationally expensive.

An example of this is described in U.S. patent application Ser. No. 09/611,220, incorporated in entirety with all figures by reference, which uses a variation on the SBS technique. In this method, a Genetic Algorithm (please see section on classifiers) is used in combination with a neural network to create and select child features based upon a fitness ranking that takes into effect multiple performance measures such as sensitivity and specificity. Only top-ranked child features are used in iterating the algorithm forward.

SFFS

The SFS algorithm suffers from a so-called nesting effect. That is, once a feature has been chosen, there is no way for it to be discarded. To overcome this problem, the sequential forward floating algorithm (SFFS) was proposed. SFFS is an exponential cost algorithm that operates in a sequential manner. In each selection step SFFS performs a forward step followed by a variable number of backward ones. In essence, a feature is first unconditionally added and then features are removed as long as the generated subsets are the best among their respective size. The algorithm is so-called because it has the characteristic of floating around a potentially good solution of the specified size.

E-RFE

The Recursive Feature Elimination (RFE) is a well-known feature selection method for support vector machines (SVMs, please see section on classifiers). As a brief overview, a SVM realizes a classification function

$$f ( x ) = {\sum }_{i = 1}^{N} {α}_{i} {γ}_{i} K ( {x}_{i} , x ) + b ,$$

where the coefficients α=(αᵢ)and b are obtained by training over a set of examples S={(xᵢ, yᵢ} I=1, . . . , N, xᵢ ε Rⁿ, yᵢ ε {−1, 1} and) K(xᵢx) is the chosen kernel. In the linear case, the SVM expansion defines the hyperplane

$$f ( x ) = 〈 w , x 〉 + b , with w = {\sum }_{i = 1}^{N} {α}_{i} {γ}_{i} {x}_{i} .$$

The idea is to define the importance of a feature for a SVM in terms of its contribution to a cost function J (α). At each step of the RFE procedure, a SVM is trained on the given data set, J is computed and the feature less contributing to J is discarded. In the case of linear SVM, the variation due to the elimination of the i-th feature is δJ(i)=wᵢ²; in the non linear case, δJ(i)=½α^{t}Z\tilde{α}½α^{t}Z(−i) αwhere Z_{i,j}=yᵢyⱼ K (xᵢ, xⱼ). The heavy computational cost of RFE is a function of the number of variables, as another SVM must be trained each time a variable is removed. In the standard RFE algorithm we would eliminate just one of the many features corresponding to a minimum weight, while it would be convenient to remove all of them at once. We will go further in the instant invention by developing an ad hoc strategy for an elimination process based on the structure of the weight distribution. This strategy was first described by Furlanello (24). We introduce an entropy function H as a measure of the weight distribution. To compute the entropy, we split the range of the weights, normalized in the unit interval, into nᵢₙₜ intervals (with nᵢₙₜ=\sqrt{#R}), and we compute for each interval the relative frequencies

$${p}_{i} = \frac{£ δ J ( i )}{£ R} , i = 1 , … , {n}_{int}$$

Entropy is then defined as the following function:

$$H = - {\sum }_{i = 1}^{{n}_{int}} {p}_{i} {log}_{2} {p}_{i}$$

The following inequality immediately descends from the definition of entropy: 0≦H≦log₂nᵢₙₜ, the two bounds corresponding to the situations:
H=0; or all the weights lie in one interval;
H=log₂nᵢₙₜ; or all the intervals contain the same number of weights.
   The new entropy-based RFE (E-RFE) algorithm eliminates chunks of features at every loop, with two different procedures applied for lower or higher values of H. The distinction is needed to remove many features that have a similar (low) weight while preserving the residual distribution structure, and also allowing for differences between classification problems. E-RFE has been shown to speed up RFE by a factor of 100.

URG

One filter method especially suited for ordinal data has been developed recently by the authors of the instant invention, and offers clearly interpretable results on such data. The feature selection aspect, tentatively named URG, or Universal Regressor Gauge, is a general method for scoring and ranking the predictive sensitivity of input variables by fitting the gauge, or the scaling, on each of the input variables subject to both predictive accuracy of a nonparametric regression, and a penalty on the L1 norm of the vector of scaling parameters. The result is a sampled-gradient local minimum solution that does not require assumptions of linearity or exhaustive power-set sampling of subsets of variables. The approach penalizes the gauge θ, or the set of scaling parameters (θ₁, θ₂, . . . , θₙ), applied to each of the input variables. The authors of the instant invention generalized this method to potentially nonlinear, nonparametric models of arbitrary complexity using a kernel-based nonparametric regressor. The penalty on the gauge is regularized by a coefficient □ that is scanned across a range of values to put progressively more downward pressure on the scaling parameters, forcing the scale (and the resulting significance in distance-based regression) downward first on those variables that can be most easily eliminated without sacrificing accuracy. Because this process is analog in the state-space of the gauge, nonlinear interactions between subsets can be investigated in a continuous manner, even if the variables themselves are discrete-valued.

Other FSAs complentated, but not limited to, to be used in the instant invention include HITON Markov Blankets and Bayesian filters.

Classification

The fourth step in the predictor-building process is classification. In the supervised learning task, one is given a training set of labeled fixed-length feature vectors, from which to induce a classification model. This model, in turn, is used to predict the class label for a set of previously unseen instances. Thus, in building a classification model, the information about the class that is inherent in the features is of utmost importance. The dataset that the classifier is trained upon is broken up generally into three different sets: Training, Testing, and Evaluation. This is required since when using any classifier, the use of distinct subsets of the available data for training and testing is required to ensure generalizability. The parameters of the classifier are set with respect to the training data set, and judged versus competitors on the testing data set, and validated on the evaluation data set. To avoid over-training (i.e., memorization of features in a specific data set that are not applicable in a general manner) this succession of training steps is discontinued when the error on the validation set begins to increase significantly. We use the error on the evaluation data set as an estimate of how well we can expect our classifier to perform on new testing data as it becomes available. This estimate can be measured by 10× leave-one-out-cross-validation on the evaluation set (100× in cases of low sample number), or batch evaluation on larger data sets.

Classifiers complimentated for the instant invention include, but are not limited to, neural networks, support vector machines, genetic algorithms, kernel-based methods, and tree-based methods.

Neural Networks

One tool to use construct classifiers is that of a mapping neural network. The flexibility of neural nets to generically model data is derived through a technique of “learning”. Given a list of examples of correct input/output pairs, a neural net is trained by systematically varying its free parameters (weights) to minimize its chi-squared error in modeling the training data set. Once these optimal weights have been determined, the trained net can be used as a model of the training data set. If inputs from the training data are fed to the neural net, the net output will be roughly the correct output contained in the training data. The nonlinear interpolatory ability manifests itself when one feeds the net sets of inputs for which no examples appeared in the training data. A neural net “learns” enough features of the training data set to completely reproduce it (up to a variance inherent to the training data); the trained form of the net acts as a black box that produces outputs based on the training data.

understanding comes about because SVMs extract support vectors, which as described above are the borderline cases. Exhibiting such borderline cases allow us to identify outliers, to perform data cleaning, and to detect confounding factors. In addition, the margins of the training examples (how far they are from the decision boundary) provide useful information about the relevance of input variables, and allow the selection of the most predictive variable. SVMs are often successful even with sparse data (few examples), biased data (more examples of one category), redundant data (many similar examples), and heterogeneous data (examples coming from different sources). However, they are known to work poorly on discrete data.

In another preferred embodiment of the present invention, regression techniques are used to deliver a diagnostic or prognostic prediction using the markers declared previously. These are well-known by those of ordinary skill in the art, however a short discussion follows. For more detail, one is referred to Kleinbaum et al., Applied Regression Analysis and Multivariable Methods, Third Edition, Duxbury Press, 1998.

In the discussion of weighted least squares a need was found for a method to fit Y to more than one X. Further, it is common that the response variable Y is related to more than one regressor variable simultaneously. If a valid description of the relationship between Y and any of these response variables is to be obtained, all must be considered. Also, exclusion of any important regressor variables will adversely affect predictions of Y. In general, the equation to be considered becomes
Y=b 0+b 1X1+b 2X2+ . . . +b KXK
The Xs may be any relevant regressor variables. Often one X is a (nonlinear) transformation of another. For example, X 2=In (X 1).
the within class variance at the same time. As this technique has been around for almost 70 years it is well known and widely used to build classifiers.

Unfortunately, as previously discussed, many biological datasets are not solvable using linear techniques. Therefore, one of the classifiers we use is a non-linear variant of Fisher's discriminant. This non-linearization is made possible through the use of kernel functions, a “trick” that is borrowed from support vector machines (Boser et al., 1992). Kernel functions represent a very principled and elegant way of formulating non-linear algorithms, and the findings that are derived from using them have clear and intuitive interpretations.

In the KFD technique (Mika, 1999), one first maps the data into some feature space F through some non-linear mapping Φ. One then computes Fisher's linear discriminant in this feature space, thus implicitly yielding a non-linear discriminant in input space. In a methodology similar to SVMs, this mapping is defined in terms of a kernel function k(x,y)=(Φ(x)·Φ(y)). The training examples (i.e. the data vector containing all marker values for each patient) can in turn be expanded in terms of this kernel function as well. From this relationship one can write a formulation of the between and within class variance in terms of dot products of the kernel function and training patterns and thus find Fisher's linear discriminant in F by maximizing the ratio of these two quantities.

In another preferred embodiment of the present invention, an algorithm using Bayesian learning is trained to deliver a diagnostic or prognostic prediction using the markers declared previously. See Pearl, J. (1988). Probabilistic Reasoning in Intelligent Systems: networks of plausible inference, Morgan Kaufmann, for an overview of Bayesian learning.

While Bayesian networks (BNs) are powerful tools for knowledge representation and inference under conditions of uncertainty, they were not

Application of calculus leads to three equations whose solution requires an iterative technique. For all but the simplest of cases, solving nonlinear least squares problems involves use of computer-based algorithms. A multitude of such algorithms exist emphasizing the number of problems whose valid solution requires the nonlinear least squares technique.

Several variations of nonlinear regression exist, which one of ordinary skill in the art will be aware. One preferred case in the present invention is the use of deterministic greedy algorithms for building sparse nonlinear regression models from observational data. In this embodiment, the objective is to develop efficient numerical schemes for reducing the training and runtime complexities of nonlinear regression techniques applied to massive datasets. In the spirit of Natarajan's greedy algorithm (Natarajan, 1995), the procedure is to iteratively minimize a loss function subject to a specified constraint on the degree of sparsity required of the final model or an upper bound on the empirical error. There exist various greedy criteria for basis selection and numerical schemes for improving the robustness and computational efficiency of these algorithms.

In another preferred embodiment of the present invention, a kernel-based method is trained to deliver a diagnostic or prognostic prediction using the markers declared previously. One such method is Kernel Fisher's Discriminant (KFD). Fisher's discriminant (Fisher, 1936) is a technique to find linear functions that are able to discriminate between two or more classes. Fisher's idea was to look for a direction w that separates the class means values well (when projected onto the found direction) while achieving a small variance around these means. The hope is that it is easy to differentiate between either of the two classes from this projection with a small error. The quantity measuring the difference between the means is called between class variance and the quantity measuring the variance around these class means is called within class variance, respectively. The goal is to find a direction that maximizes the between class variance while minimizing

When dealing with multiple linear regression, fits to data are no longer lines. For example, with K=2, the resulting fit would describe a plane in three dimensional space with “slopes” bhat 1 and bhat 2intersecting the Y axis at bhat 0. Beyond K=2 the resulting fit becomes difficult to visualize. The terminology regression surface is often used to describe a multiple linear regression fit.

Assumptions required for application of least squares methodology to multiple linear regression equations are similar to those cited for the simple linear case. For example, the true relationship between Y and the various Xs must be as given by the linear equation and the spread of the errors must be constant across values of all Xs. Also, a limit exists to the number of Xs that can be considered. Specifically, K+1 must be less than or equal to the sample size n for a unique set of bhats to be found.

In theory, least squares estimates of b 0, . . . , b K are found just as in the simple linear case. The estimates bhat 0, . . . , bhat K are the solution from minimizing sum (Yi−b0−b1X1i− . . . −bkXki)sup2.

The description of the resulting equations and associated summary statistics is best made using matrix algebra. The computations are best carried out using a computer.

The relationship between Y and X or Y and several Xs is not always linear in form despite transformations that can be applied to resulted in a linear relationship. In some instances such a transformation may not exist and in others theoretical concerns may require analysis to be carried out with the untransformed equation.

Least squares methodology can be used to solve nonlinear regression problems. For the above equation the least squares estimates of the parameters would be the solution of the minimization of sum(W−A (1−e sup Bt )sup C)sup 2

GAs have demonstrated substantial improvement over a variety of random and local search methods. This is accomplished by their ability to exploit accumulating information about an initially unknown search space in order to bias subsequent search into promising subspaces. Since GAs are basically a domain independent search technique, they are ideal for applications where domain knowledge and theory is difficult or impossible to provide.

SVMs

The key idea behind support vector machines (SVMs, Vapnik, 1995) is to map input vectors (i.e., patient-specific data) into a high dimensional space, and to construct in that space hyperplanes with a large margin. These hyperplanes can be thought of as boundaries separating the categories of the dataset, in this case response and non-response. The support vector machine solution proposes to find the hyperplane separating the classes. This plane is determined by the parameters of a decision function, which is used for classification. The SVM is based on the fact that there is a unique separating hyperplane that maximizes the margin between the classes.

The task of finding the hyperplane is reduced to minimizing the Lagrangian, a function of the margin and constraints associated with each input vector. The constraints depend only on the dot product of an input element and the solution vector. In order to minimize the Langrangian, the Lagrange multipliers must either satisfy those constraints or be exactly zero. Elements of the training set for which the constraints are satisfied are the so-called support vectors. The support vectors parameterize the decision function and lie on the boundaries of the margin separating the classes.

In many cases, SVMs are typically more accurate, give greater data understanding, and are more robust than other machine learning methods. Data

Neural networks typically have a number of ad hoc parameters, such as selection of the number of hidden layers, the number of hidden-layer neurons, parameters associated with the learning or optimization technique used, and in many cases they require a validation set for a stopping criterion. In addition, neural network weights are trained iteratively, producing problems with convergence to local minima. We have developed several types of neural networks that solve these problems. Our solutions involve nonlinearly transforming the input pattern fed into the neural network. This transformation is equivalent to feature selection (though one still needs as many inputs into the classifier) and can be quite powerful when combined with the independent feature selection techniques previously described.

Genetic Algorithms

Genetic algorithms (GAs) typically maintain a constant sized population of individual solutions that represent samples of the space to be searched. Each individual is evaluated on the basis of its overall “fitness” with respect to the given application domain. New individuals (samples of the search space) are produced by selecting high performing individuals to produce “offspring” that retain features of their “parents”. This eventually leads to a population that has improved fitness with respect to the given goal.

New individuals (offspring) for the next generation are formed by using two main genetic operators: crossover and mutation. Crossover operates by randomly selecting a point in the two selected parents gene structures and exchanging the remaining segments of the parents to create new offspring. Therefore, crossover combines the features of two individuals to create two similar offspring. Mutation operates by randomly changing one or more components of a selected individual. It acts as a population perturbation operator and is a means for inserting new information into the population. This operator prevents any stagnation that might occur during the search process.

considered as classifiers until the discovery that Naïve-Bayes, a very simple kind of BNs that assumes the attributes are independent given the class node, are surprisingly effective. See Langley, P., Iba, W. and Thompson, K. (1992). An analysis of Bayesian classifiers. In Proceedings of AAAI-92 pp. 223-228.

A Bayesian network B is a directed acyclic graph (DAG), where each node N represents a domain variable (i.e., a dataset attribute), and each arc between nodes represents a probabilistic dependency, quantified using a conditional probability distribution (CP table) for each node n.sub.i. A BN can be used to compute the conditional probability of one node, given values assigned to the other nodes; hence, a BN can be used as a classifier that gives the posterior probability distribution of the class node given the values of other attributes. A major advantage of BNs over many other types of predictive models, such as neural networks, is that the Bayesian network structure represents the inter-relationships among the dataset attributes. One of ordinary skill in the art can easily understand the network structures and if necessary modify them to obtain better predictive models. By adding decision nodes and utility nodes, BN models can also be extended to decision networks for decision analysis. See Neapolitan, R. E. (1990), Probabilistic reasoning in expert systems: theory and algorithms, John Wiley& Sons.

Applying Bayesian network techniques to classification involves two sub-tasks: BN learning (training) to get a model and BN inference to classify instances. Learning BN models can be very efficient. As for Bayesian network inference, although it is NP-hard in general (See for instance Cooper, G. F. (1990) Computational complexity of probabilistic inference using Bayesian belief networks, In Artificial Intelligence, 42 (pp. 393-405).), it reduces to simple multiplication in a classification context, when all the values of the dataset attributes are known.

The two major tasks in learning a BN are: learning the graphical structure, and then learning the parameters (CP table entries) for that structure. One skilled in the art knows it is easy to learn the parameters for a given structure that are optimal for a given corpus of complete data, the only step being to use the empirical conditional frequencies from the data.

There are two ways to view a BN, each suggesting a particular approach to learning. First, a BN is a structure that encodes the joint distribution of the attributes. This suggests that the best BN is the one that best fits the data, and leads to the scoring based learning algorithms, that seek a structure that maximizes the Bayesian, MDL or Kullback-Leibler (KL) entropy scoring function. See for instance Cooper, G. F. and Herskovits, E. (1992). A Bayesian Method for the induction of probabilistic networks from data. Machine Learning, 9 (pp. 309-347). Second, the BN structure encodes a group of conditional independence relationships among the nodes, according to the concept of d-separation. See for instance Pearl, J. (1988). Probabilistic Reasoning in Intelligent Systems: networks of plausible inference, Morgan Kaufmann. This suggests learning the BN structure by identifying the conditional independence relationships among the nodes. These algorithms are referred as CI-based algorithms or constraint-based algorithms. See for instance Cheng, J., Bell, D. A. and Liu, W. (1997a). An algorithm for Bayesian belief network construction from data. In Proceedings of AI &STAT'97 (pp. 83-90), Florida.

Friedman et al. (1997) show theoretically that the general scoring-based methods may result in poor classifiers since a good classifier maximizes a different function −viz., classification accuracy. Greiner et al. (1997) reach the same conclusion, albeit via a different analysis. Moreover, the scoring-based methods are often less efficient in practice. The preferred embodiment is CI-based learning algorithms to effectively learn BN classifiers.

The present invention envisions using, but is not limited to, the following five classes of BN classifiers: Naïve-Bayes, Tree augmented Naïve-Bayes (TANs), Bayesian network augmented Naïve-Bayes (BANs), Bayesian multi-nets and general Bayesian networks (GBNs). By use of this methodology it is possible to build a predictive model of the data.

These models can be put on firm theoretical foundations of statistics and probability theory, i.e. in a Bayesian setting. The computation required for inference in these models include optimization or marginalisation over all free parameters in order to make predictions and evaluations of the model. Inference in all but the very simplest models is not analytically tractable, so approximate techniques such as variational approximations and Markov Chain Monte Carlo may be needed. Models include probabilistic kernel based models, such as Gaussian Processes and mixture models based on the Dirichlet Process.

Ensemble Networks

The final step in predictor development, assembly of committee, or ensemble, networks.It is common practice to train many different candidate networks and then to select the best, on the basis of performance on an independent validation set, for instance, and to keep this network, discarding the rest. There are two disadvantages to this approach. First, the effort involved in training the remaining networks is wasted. Second, the generalization performance on the validation set has a random component due to noise on the data, and so the network that had the best performance on the validation set might not be the one with the best performance on the new test set.

These drawbacks can be overcome by combining the networks together to form a committee. This can lead to significant improvements in the predictions on new data while involving little additional computational effort. In fact, the performance of a committee can be better than the performance of the best single network in isolation. The error due to the committee can be shown to be:
E_{COM}=1/L E_{AV}
Where L is the number of committee members and EAV the average error contributed to the prediction by a single member of the committee. Typically, some useful reduction in error is obtained, and the method is trivial to implement.

The challenging problem of integration is to decide which one(s) of the classifiers to rely on or how to combine the results produced by the base classifiers. One of the most popular and simplest techniques used is called majority voting. In the voting technique, each base classifier is considered as an equally weighted vote for that particular prediction. The classification that receives the largest number of votes is selected as the final classification (ties are solved arbitrarily). Often, weighted voting is used: each vote receives a weight, which is usually proportional to the estimated generalization performance of the corresponding classifier. Weighted Voting (WV) works usually much better than simple majority voting.

Boosting Networks

Boosting has been found to be a powerful classification technique with remarkable success on a wide variety of problems, especially in higher dimensions. It aims at producing an accurate combined classifier from a sequence of weak (or base) classifiers, which are fitted to iteratively reweighted versions of the data.

In each boosting iteration, m, the observations that have been misclassified at the previous step have their weights increased, whereas the weights are decreased for those that were classified correctly. The m^{th} weak classifier f(m) is thus forced to focus more on individuals that have been difficult to classify correctly at earlier iterations. In other words, the data is re-sampled adaptively so that the weights in the re-sampling are increased for those cases most often misclassified. The combined classifier is equivalent to a weighted majority vote of the weak classifiers.

Entropy-Based

One efficient way to construct an ensemble of diverse classifiers is to use different feature subsets. To be effective, an ensemble should consist of high-accuracy classifiers that disagree on their predictions. To measure the disagreement of a base classifier and the whole ensemble, we calculate the diversity of the base classifier over the instances of the validation set as an average difference in classifications of all possible pairs of classifiers including the given one. A measure of this is based on the concept of entropy:

$$div\_ent = \frac{1}{N} {\sum }_{l = 1}^{N} {\sum }_{k = 1}^{l} - \frac{{N}_{k}^{l}}{S} \cdot log ( \frac{{N}_{k}^{l}}{S} )$$

where N is the number of instances in the data set, S is the number of base classifiers, l is the number of classes, and Nₖ^{l} is the number of base classifiers that assign instance i to class k.

### BRIEF DESCRIPTION OF THE DRAWINGS

In the following, the invention will be explained in further detail with reference to the drawings, in which:

FIG. 1 is a list illustrating SNPs genotyped for patients on the drug citalopram;

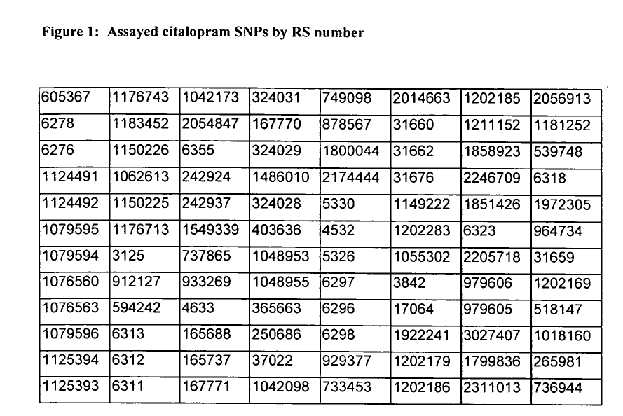

FIG. 2 is a list showing top linear correlating SNPs with response for patients on the drug citalopram;

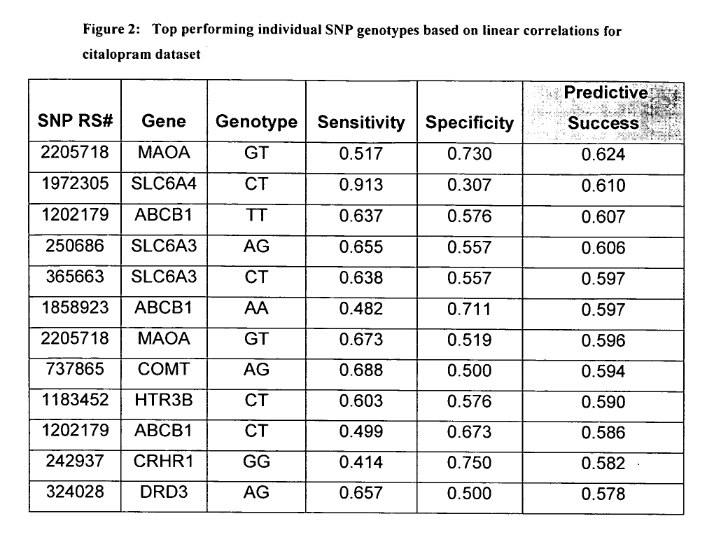

FIG. 3 is a graph illustrating neural network predictability of an aggregate of linear correlates;

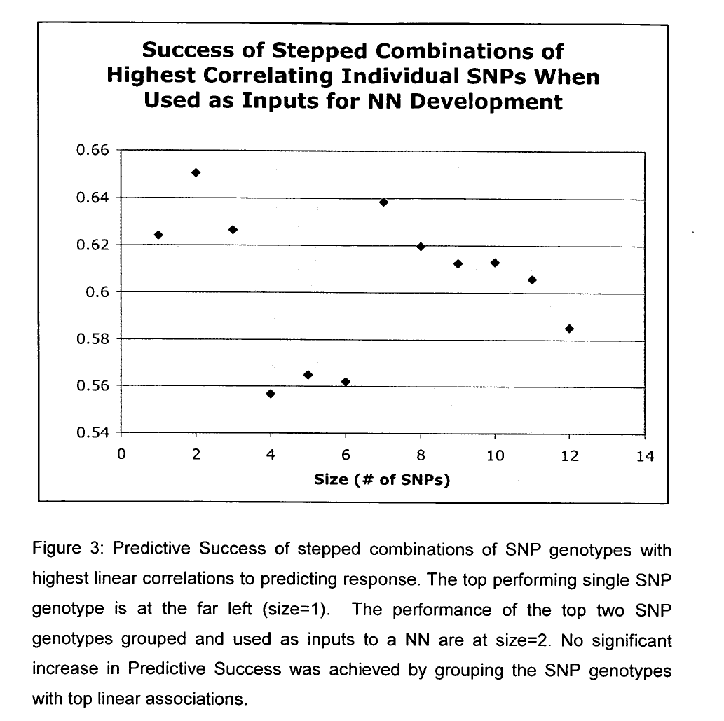

FIG. 4 is a graph illustrating temporal correlations between HAM-D and CGI-S;

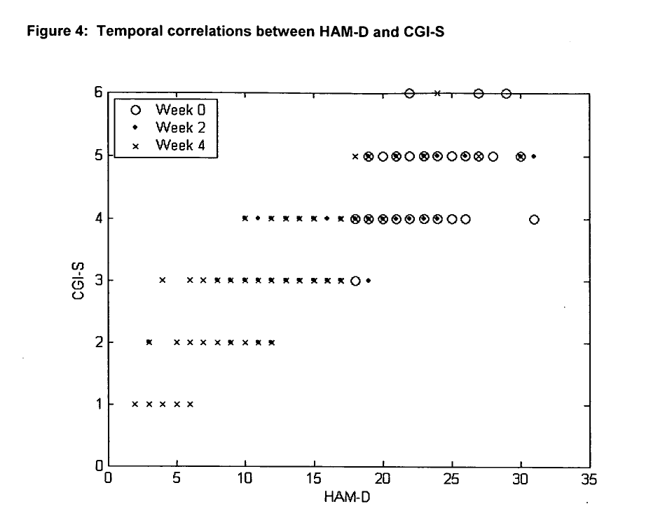

FIG. 5 is a graph illustrating cumulative probability distributions of depression measure ratios between outcome and baseline;

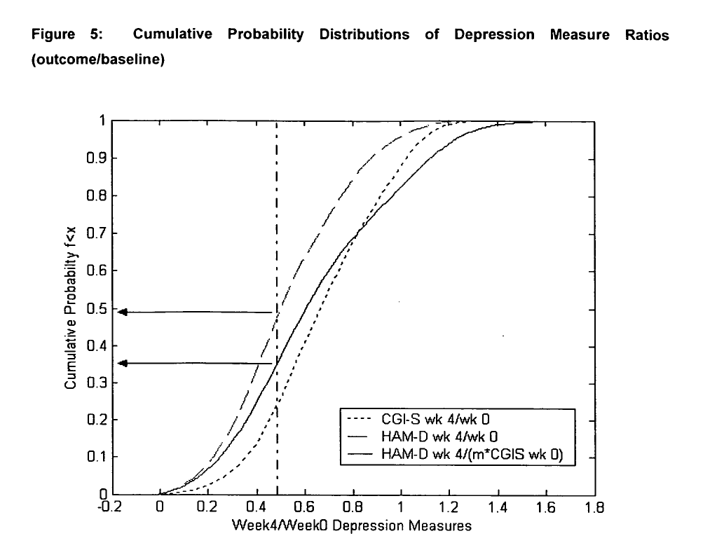

FIG. 6 is a list showing model SNPs indicative of predicting response or nonresponse in patients taking citalopram;

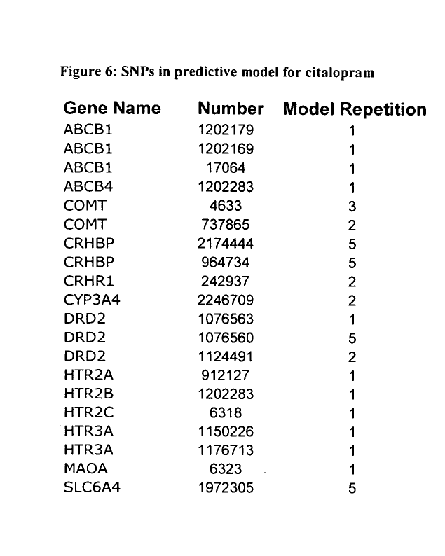

FIG. 7 is a chart illustrating model classification performance for predicting response or nonresponse in patients taking citalopram;

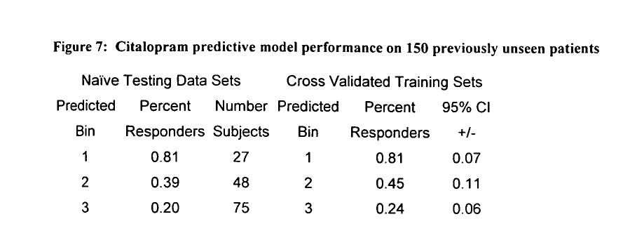

FIG. 8 is a list showing model SNPs indicative of predicting response or nonresponse in patients taking paroxetine;

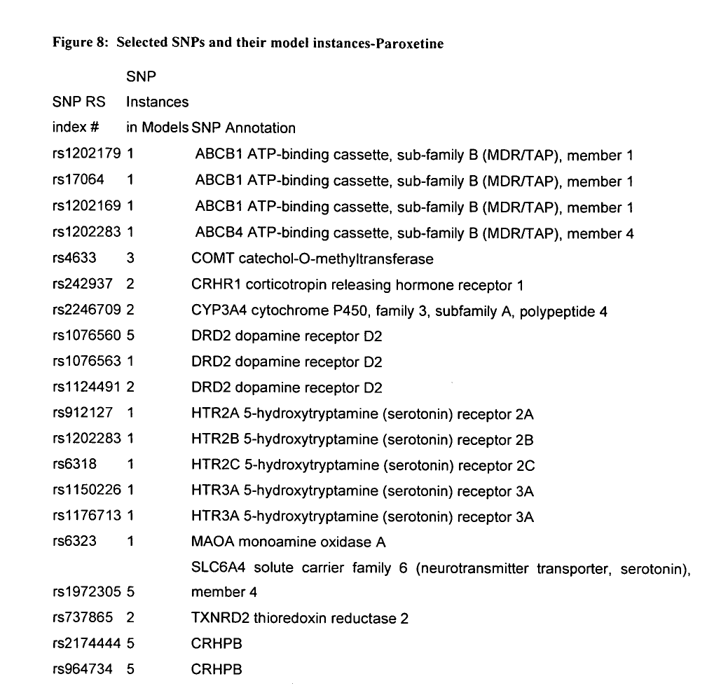

FIG. 9 is a chart illustrating model classification performance for predicting response or nonresponse in patients taking paroxetine;

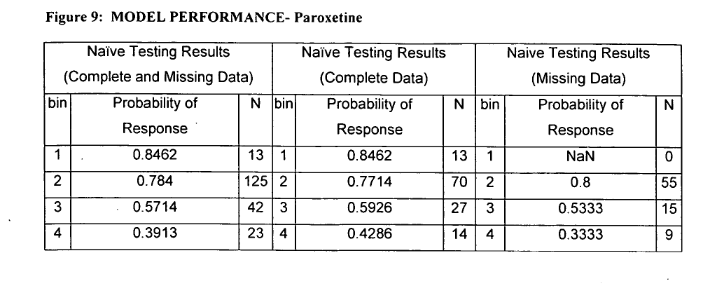

FIG. 10 is a list showing model SNPs indicative of predicting response or nonresponse in patients taking paroxetine with a probabilistic bayes network;

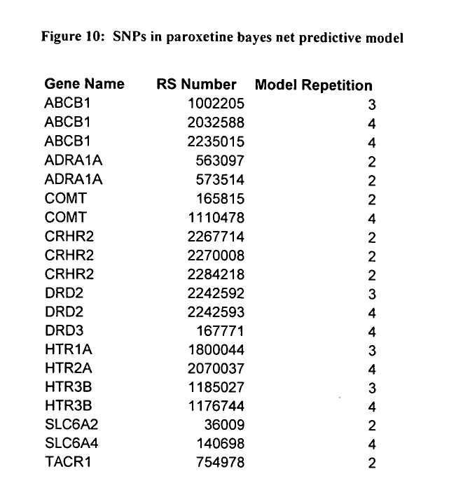

FIG. 11 is a chart illustrating model classification performance for predicting response or nonresponse in patients taking paroxetine with a probabilistic bayes network;

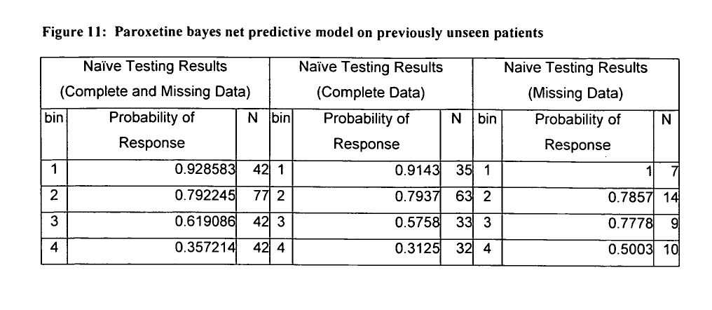

FIG. 12 is a list showing model SNPs indicative of predicting response or nonresponse in patients taking citalopram with a probabilistic bayes network;

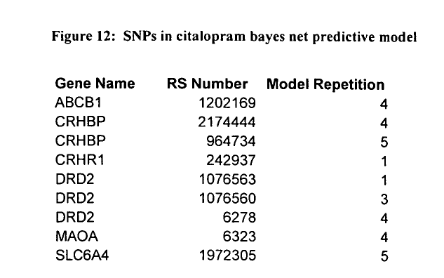

FIG. 13 is a chart illustrating model classification performance for predicting response or nonresponse in patients taking citalopram with a probabilistic bayes network;

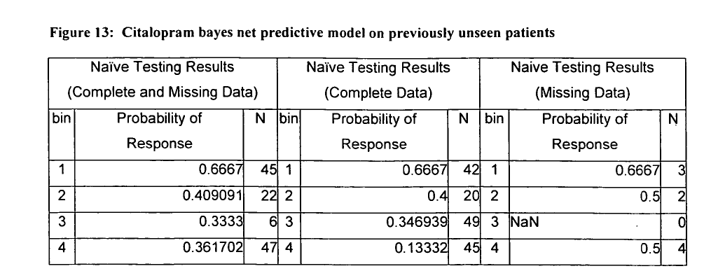

While the invention has been described and exemplified in sufficient detail for those skilled in this art to make and use it, various alternatives, modifications, and improvements should be apparent without departing from the spirit and scope of the invention.

One skilled in the art readily appreciates that the present invention is well adapted to carry out the objects and obtain the ends and advantages mentioned, as well as those inherent therein. The examples provided herein are representative of preferred embodiments, are exemplary, and are not intended as limitations on the scope of the invention. Modifications therein and other uses will occur to those skilled in the art. These modifications are encompassed within the spirit of the invention and are defined by the scope of the claims.

It will be readily apparent to a person skilled in the art that varying substitutions and modifications may be made to the invention disclosed herein without departing from the scope and spirit of the invention.

All patents and publications mentioned in the specification are indicative of the levels of those of ordinary skill in the art to which the invention pertains. All patents and publications are herein incorporated by reference to the same extent as if each individual publication was specifically and individually indicated to be incorporated by reference.

The invention illustratively described herein suitably may be practiced in the absence of any element or elements, limitation or limitations which is not specifically disclosed herein. Thus, for example, in each instance herein any of the terms “comprising”, “consisting essentially of” and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

Other embodiments are set forth within the following claims.

1. A method of determining response to a pharmaceutical agent for depression, the method comprising: correlating (i) a mutational burden at one or more nucleotide positions in the ABCB1, ABCB4, COMT, CRHR1, CRHBP, CYP3A4, DRD1, DRD2, DRD3, HRT1A, HTR1B, HTR2A, HTR3A, HTR3B, DRD3, MAOA, MAOB, SLC6A3, HTR2A, HTR2B, HTR2C, HTR3A, HTR3B, MAOA, MAOB, NR3C1, SLC6A2 SLC6A3, SLC6A4, TAC1, TACR1 or TPH genes in a sample from the subject with (ii) the mutational burden at one or more corresponding nucleotide positions in a control sample with known response outcome, and therefrom identifying the probability of response to said pharmaceutical agent.

2. A method according to claim 1 wherein the mutational burden relates to a mutation in the ABCB1 gene at nucleotide position given by the RS #17064, 1002205, 2032588, 2235015, 2235040, 2235048, 1202169, 1202179, or 1202180; in the ABCB4 gene at nucleotide position given by the RS#1202283; in the ADRA1A gene at nucleotide position given by the RS#563097 or 573514; in the ADRB2 gene at nucleotide position given by the RS#1032713 or 1042713; in the COMT gene at nucleotide position given by the RS#4633, 165815, 737865 or 1110478; in the CRHR1 gene at nucleotide position given by the RS#242937; in the CRHR2 gene at nucleotide position given by the RS#3802, 2267714, 2270008, or 2284218; in the CYP3A4 gene at nucleotide position given by the RS#2246709; in the CRHBP gene at nucleotide position given by the RS#2174444 or 964734; in the DRD2 gene at nucleotide position given by the RS#1076560, 1076563, 1124491, 1079595, 2242592, or 2242593; in the DRD3 gene at nucleotide position given by the RS#167771; in the HTR1A gene at nucleotide position given by the RS#1800044; in the HTR2A gene at nucleotide position given by the RS#912127 or 2070037; in the HTR3A gene at nucleotide position given by the RS#1150226. or 1176713; in the HTR1B gene at nucleotide position given by the RS#6298; in the HTR2B gene at nucleotide position given by the RS#1202283; in the HTR3B gene at nucleotide position given by the RS#1183452, 1185027, 1176743, or 1176744; in the HTR2C gene at nucleotide position given by the RS#6318; in the MAOA gene at nucleotide position given by the RS#979606, 6323, or 2205718; in the SLC6A2 gene at nucleotide position given by the RS#36009 or 42460; in the SLC6A3 gene at nucleotide position given by the RS#250686 or 365663; in the SLC6A4 gene at nucleotide position given by the RS#140698 or 1972305; in the TACR1 gene at nucleotide position given by the RS#975664, 737679, or 754978; any mutations in linkage disequilibrium with said stated mutations; or combinations thereof.

3. A method according to claim 1 wherein the mutational burden relates to a mutation in the MAOB gene at nucleotide position given by the RS #1181252 or 6305; in the ABCB1 gene at nucleotide position given by the RS#3842, 1858923 or 1202179; in the ABCB4 gene at nucleotide position given by the RS#1149222 or 594242; in the COMT gene at nucleotide position given by the RS#737865; in the CRHR1 gene at nucleotide position given by the RS#242937; in the CRHBP gene at nucleotide position given by the RS#2174444 or 964734; in the DRD2 gene at nucleotide position given by the RS#6278; in the DRD3 gene at nucleotide position given by the RS#167771 or 324028; in the HTR3A gene at nucleotide position given by the RS#1150226; in the HTR3B gene at nucleotide position given by the RS#1183452 in the MAOA gene at nucleotide position given by the RS#979606 or 2205718; in the SLC6A3 gene at nucleotide position given by the RS#250686 or 365663; in the SLC6A4 gene at nucleotide position given by the RS#1972305; any mutations in linkage disequilibrium with said stated mutations; or combinations thereof.

4. A method according to claim 1 wherein the mutational burden is comprised of one or more of the following combinations in vertical column format:

| SNP RS# | GENE | Genotype | SNP RS# | GENE | Genotype |
|---|---|---|---|---|---|
| 1181252 | MAOB | AG | 1181252 | MAOB | AG |
| 1972305 | SLC6A4 | CT | 1972305 | SLC6A4 | CT |
| 979606 | MAOA | TT | 979606 | MAOA | TT |
| 242937 | CRHR1 | AG | 242937 | CRHR1 | AG |
| 964734 | CRHBP | GG | 964734 | CRHBP | GG |
| 324028 | DRD3 | AG | 324028 | DRD3 | AG |
| 2174444 | CRHBP | TT | 2174444 | CRHBP | TT |
| 167771 | DRD3 | AG | 167771 | DRD3 | AG |
| 1150226 | HTR3A | AG | 1150226 | HTR3A | AG |
| 1149222 | ABCB4 | GT | 594242 | ABCB4 | CG |
| 6355 | MAOA | CG | 3842 | ABCB1 | CT |
| 2174444 | CRHBP | CT | 6355 | MAOA | CG |
| 6278 | DRD2 | AA | 2174444 | CRHBP | CC |
| — | — | — | 6278 | DRD2 | AA |

Or

| SNP rs#/genotype | Gene | SNP rs# | Gene |
|---|---|---|---|
| 1202169/2 | ABCB1 | 4633 | COMT |
| 1055302/1 | ABCB1 | 242937 | CRHR1 |
| 165688/2 | COMT | 2246709 | CYP3A4 |
| 964734/1 | CRHBP | 265981 | DRD1 |
| 1062613/3 | HTR3A | 1076560 | DRD2 |
| 979606/3 | MAOA | 1076563 | DRD2 |
| 2311013/3 | MAOB | 167770 | DRD3 |
| 2056913/2 | MAOB | 324029 | DRD3 |
| 1972305/2 | SLC6A4 | 1800044 | HTR1A |
| | | 1549339 | HTR2B |
| | | 1150226 | HTR3A |
| | | 979605 | MAOA |
| | | 979606 | MAOA |
| | | 1181252 | MAOB |
| | | 2056913 | MAOB |
| | | 365663 | SLC6A3 |
| | | 403636 | SLC6A3 |
| | | 6355 | SLC6A4 |
| | | 1972305 | SLC6A4 |

Or

| 1 | 2 | 3 | 4 | 5 |
|---|---|---|---|---|
| MAOA 979606 | MAOA 979606 | CRHR1 242924 | CRHR1 242924 | MAOA 979606 |
| SLC6A4 1972305 | SLC6A4 1972305 | CRHR2 929377 | CRHR2 929377 | SLC6A4 1972305 |
| ABCB1 1202169 | ABCB1 1202169 | MAOA 979606 | CYP3A4 2246709 | ABCB1 1202169 |
| ABCB1 1055302 | ABCB1 1055302 | HTR1B 6296 | HTR3A 1062613 | ABCB1 1055302 |
| CRHBP 964734 | CRHBP 964734 | MAOB 1181252 | SLC6A3 1042098 | CRHBP 964734 |
| COMT 165688 | COMT 165688 | SLC6A4 1972305 | CRHBP 2174444 | COMT 165688 |
| MAOB 2311013 | MAOB 2311013 | ABCB1 1202186 | CYP3A4 1851426 | MAOB 2311013 |
| | MAOB 2056913 | DRD2 6278 | | MAOB 2056913 |
| | HTR3A 1062613 | ABCB1 1202169 | | |
| 6 | 7 | 8 | 9 | 10 |
| CRHR1 242924 | CRHR1 242924 | MAOA 979606 | MAOA 979606 | MAOA 979606 |
| CRHR2 929377 | CRHR2 929377 | SLC6A4 1972305 | SLC6A4 1972305 | SLC6A4 1972305 |
| CYP3A4 2246709 | MAOA 979606 | ABCB1 1202169 | ABCB1 1202169 | ABCB1 1202169 |
| HTR3A 1062613 | HTR1B 6296 | ABCB1 1055302 | ABCB1 1055302 | ABCB1 1055302 |
| SLC6A3 1042098 | MAOB 1181252 | CRHBP 964734 | CRHBP 964734 | CRHBP 964734 |
| CRHBP 2174444 | SLC6A4 1972305 | MAOB 736944 | MAOB 736944 | MAOB 736944 |
| | ABCB1 1202186 | HTR2A 6313 | HTR2A 6313 | HTR2A 6313 |
| | DRD2 6278 | MAOB 2311013 | MAOB 2311013 | MAOB 2311013 |
| | MAOB 1799836 | | CYP3A4 1851426 | |
| 11 | 12 | 13 | 14 | 15 |
| CRHR1 242924 | MAOA 979606 | CRHR1 242924 | CRHR1 242924 | MAOA 979606 |
| CRHR2 929377 | SLC6A4 1972305 | CRHR2 929377 | CRHR2 929377 | SLC6A4 1972305 |
| MAOA 979606 | ABCB1 1202169 | MAOA 979606 | MAOA 979606 | ABCB1 1202169 |
| HTR1B 6296 | ABCB1 1055302 | HTR1B 6296 | HTR1B 6296 | ABCB1 1055302 |
| MAOB 1181252 | CRHBP 964734 | MAOB 1181252 | MAOB 1181252 | CRHBP 964734 |
| SLC6A4 1972305 | MAOB 736944 | SLC6A4 1972305 | SLC6A4 1972305 | MAOB 736944 |
| ABCB1 1202186 | HTR2A 6313 | MAOB 1799836 | ABCB1 1202186 | HTR2A 6313 |
| SLC6A3 37022 | MAOB 2311013 | CYP3A4 2246709 | DRD2 6278 | MAOB 2311013 |
| | COMT 165688 | | | COMT 165688 |
| | CYP3A4 2246709 | | | |
| 16 | 17 | 18 | 19 | 20 |
| CRHR1 242924 | CRHR1 242924 | MAOA 979606 | MAOA 979606 | CRHR1 242924 |
| CRHR2 929377 | CRHR2 929377 | SLC6A4 1972305 | SLC6A4 1972305 | CRHR2 929377 |
| MAOA 6323 | MAOA 979606 | ABCB1 1202169 | ABCB1 1202169 | MAOA 979606 |
| ABCB1 1858923 | HTR1B 6296 | ABCB1 1055302 | ABCB1 1055302 | HTR1B 6296 |
| CYP3A4 2246709 | MAOB 1181252 | CRHBP 964734 | CRHBP 964734 | MAOB 1181252 |
| MAOA 6355 | SLC6A4 1972305 | MAOB 736944 | MAOB 736944 | SLC6A4 1972305 |
| HTR2B 1549339 | ABCB1 1202186 | HTR2A 6313 | HTR2A 6313 | MAOA 6323 |
| MAOB 2311013 | MAOB 2311013 | MAOB 2311013 | MAOB 2311013 | CYP3A4 2246709 |
| | | COMT 165688 | COMT 165688 | |
| | | HTR2A 3125 | HTR2A 3125 | |
| | | MAOB 1181252 | MAOB 1181252 | |
| | | HTR2A 6312 | HTR2A 6312 | |
| | | MAOA 6355 | MAOA 6355 | |
| | | | MAOB 2311013 | |
| 21 | 22 | 23 | 24 | 25 |
| CRHR1 242924 | CRHR1 242924 | MAOA 979606 | CRHR1 242924 | CRHR1 242924 |
| CRHR2 929377 | CRHR2 929377 | SLC6A4 1972305 | CRHR2 929377 | CRHR2 929377 |
| MAOA 979606 | MAOA 979606 | ABCB1 1202169 | MAOA 6323 | MAOA 979606 |
| HTR1B 6296 | HTR1B 6296 | ABCB1 1055302 | ABCB1 1858923 | HTR1B 6296 |
| MAOB 1181252 | MAOB 1181252 | CRHBP 964734 | CYP3A4 2246709 | MAOB 1181252 |
| SLC6A4 1972305 | SLC6A4 1972305 | MAOB 736944 | DRD2 6278 | SLC6A4 1972305 |
| MAOA 6355 | ABCB1 1202186 | HTR2A 6313 | | MAOA 6355 |
| CYP3A4 2246709 | | MAOB 2311013 | | ABCB1 1202169 |
| | | COMT 165688 | | |
| | | HTR2A 3125 | | |
| | | MAOB 1181252 | | |
| | | HTR2A 6312 | | |
| | | MAOA 6355 | | |
| | | SLC6A3 403636 | | |
| 26 | 27 | 28 | 29 | 30 |
| MAOA 979606 | CRHR1 242924 | MAOA 979606 | CRHR1 242924 | CRHR1 242924 |
| SLC6A4 1972305 | CRHR2 929377 | SLC6A4 1972305 | CRHR2 929377 | CRHR2 929377 |
| ABCB1 1202169 | MAOA 6323 | ABCB1 1202169 | CYP3A4 2246709 | MAOA 6323 |
| ABCB1 1055302 | ABCB1 1858923 | ABCB1 1055302 | HTR3A 1062613 | ABCB1 1858923 |
| CRHBP 964734 | CYP3A4 2246709 | CRHBP 964734 | SLC6A3 1042098 | CYP3A4 2246709 |
| MAOB 736944 | DRD2 1125394 | MAOB 736944 | CRHBP 2174444 | MAOA 6355 |
| HTR2A 6313 | HTR2B 1549339 | HTR2A 6313 | HTR2B 1549339 | HTR2B 1549339 |
| MAOB 2311013 | | MAOB 2311013 | | |
| COMT 165688 | | COMT 165688 | | |
| DRD2 6276 | | HTR2A 3125 | | |
| HTR2A 3125 | | MAOB 1181252 | | |
| | | HTR2A 6312 | | |
| | | MAOA 6355 | | |
| | | SLC6A3 403636 | | |
| 31 | 32 | 33 | 34 | 35 |
| MAOA 979606 | MAOA 979606 | CRHR1 242924 | MAOA 979606 | CRHR1 242924 |
| SLC6A4 1972305 | SLC6A4 1972305 | CRHR2 929377 | SLC6A4 1972305 | CRHR2 929377 |
| ABCB1 1202169 | ABCB1 1202169 | MAOA 979606 | ABCB1 1202169 | CYP3A4 2246709 |
| ABCB1 1055302 | ABCB1 1055302 | HTR1B 6296 | ABCB1 1055302 | HTR3A 1062613 |
| CRHBP 964734 | CRHBP 964734 | MAOB 1181252 | CRHBP 964734 | SLC6A3 1042098 |
| MAOB 736944 | MAOB 736944 | SLC6A4 1972305 | MAOB 736944 | CRHBP 2174444 |
| HTR2A 6313 | HTR2A 6313 | MAOA 6323 | | CYP3A4 1851426 |
| MAOB 2311013 | MAOB 2311013 | CYP3A4 2246709 | | HTR3A 1150226 |
| COMT 165688 | COMT 165688 | HTR2A 594242 | | |
| HTR2A 3125 | HTR1A 1800044 | | | |
| MAOB 1181252 | | | | |
| HTR2A 6312 | | | | |
| MAOA 6355 | | | | |
| SLC6A3 403636 | | | | |
| SLC6A3 1042098 | | | | |
| 36 | 37 | 38 | 39 | 40 |
| CRHR1 242924 | CRHR1 242924 | CRHR1 242924 | CRHR1 242924 | CRHR1 242924 |
| CRHR2 929377 | CRHR2 929377 | CRHR2 929377 | CRHR2 929377 | CRHR2 929377 |
| CYP3A4 2246709 | MAOA 979606 | CYP3A4 2246709 | MAOA 6323 | MAOA 979606 |
| HTR3A 1062613 | HTR1B 6296 | HTR3A 1062613 | ABCB1 1858923 | HTR1B 6296 |
| HTR2A 3125 | MAOB 1181252 | SLC6A3 1042098 | CYP3A4 2246709 | MAOB 1181252 |
| ABCB4 1202283 | SLC6A4 1972305 | CRHBP 2174444 | DRD2 1125394 | SLC6A4 1972305 |
| | MAOB 1181252 | ABCB1 1202179 | | MAOA 6355 |
| 41 | 42 | 43 | 44 | 45 |
| CRHR1 242924 | MAOA 979606 | CRHR1 242924 | CRHR1 242924 | CRHR1 242924 |
| CRHR2 929377 | SLC6A4 1972305 | CRHR2 929377 | CRHR2 929377 | CRHR2 929377 |
| MAOA 6323 | ABCB1 1202169 | MAOA 6323 | MAOA 6323 | MAOA 979606 |
| ABCB1 1858923 | ABCB1 1055302 | ABCB1 1858923 | ABCB1 1858923 | HTR1B 6296 |
| CYP3A4 2246709 | CRHBP 964734 | CYP3A4 2246709 | CYP3A4 2246709 | MAOB 1181252 |
| MAOB 2311013 | MAOB 736944 | DRD2 1124491 | HTR2A 6311 | SLC6A4 1972305 |
| | HTR2A 6313 | | | ABCB1 1202186 |
| | MAOB 2311013 | | | HTR1B 6298 |
| | COMT 165688 | | | CYP3A4 2246709 |
| | HTR1A 1800044 | | | |
| | COMT 4633 | | | |
| 46 | 47 | 48 | 49 | 50 |
| CRHR1 242924 | CRHR1 242924 | CRHR1 242924 | MAOA 979606 | MAOA 979606 |
| CRHR2 929377 | CRHR2 929377 | CRHR2 929377 | SLC6A4 1972305 | SLC6A4 1972305 |
| MAOA 979606 | MAOA 979606 | MAOA 6323 | ABCB1 1202169 | ABCB1 1202169 |
| HTR1B 6296 | HTR1B 6296 | ABCB1 1858923 | ABCB1 3842 | ABCB1 1055302 |
| MAOB 1181252 | MAOB 1181252 | CYP3A4 2246709 | CRHBP 964734 | CRHBP 964734 |
| SLC6A4 1972305 | SLC6A4 1972305 | CRHR2 2014663 | MAOA 2205718 | MAOB 736944 |
| ABCB1 1202186 | | | SLC6A3 365663 | HTR2A 6313 |
| DRD2 6278 | | | MAOB 1181252 | MAOB 2311013 |
| MAOB 1799836 | | | | COMT 165688 |
| CRHBP 2174444 | | | | HTR2A 3125 |

5. A method according to claim 1, wherein said correlating step comprising:
a) determining the sequence of one or more of the genes ABCB1, ABCB4, COMT, CRHR1, CRHBP, CYP3A4, DRD1, DRD2, DRD3, HRT1A, HTR1B, HTR2A, HTR3A, HTR3B, DRD3, MAOA, MAOB, SLC6A3, HTR2A, HTR2B, HTR2C, HTR3A, HTR3B, MAOA, MAOB, NR3C1, SLC6A2 SLC6A3, SLC6A4, TAC1, TACR1 or TPH from humans known to be responsive or non-responsive to anti-depression medications; b) comparing said sequence to that of the corresponding wildtype ABCB1, ABCB4, COMT, CRHR1, CRHBP, CYP3A4, DRD1, DRD2, DRD3, HRT1A, HTR1B, HTR2A, HTR3A, HTR3B, DRD3, MAOA, MAOB, SLC6A3, HTR2A, HTR2B, HTR2C, HTR3A, HTR3B, MAOA, MAOB, NR3C1, SLC6A2 SLC6A3, SLC6A4, TAC1, TACR1 or TPH genes; and c) identifying mutations in said humans which correlate with the response or non-response to anti-depressant medications, respectively.

6. A method according to claim 1, wherein said correlating step comprising:
a) determining the sequence of one or more of the genes ABCB1, ABCB4, COMT, CRHR1, CRHBP, CYP3A4, DRD1, DRD2, DRD3, HRT1A, HTR1B, HTR2A, HTR3A, HTR3B, DRD3, MAOA, MAOB, SLC6A3, HTR2A, HTR2B, HTR2C, HTR3A, HTR3B, MAOA, MAOB, NR3C1, SLC6A2 SLC6A3, SLC6A4, TAC1, TACR1 or TPH from humans known to be responsive or non-responsive to SSRI depression medications; b) comparing said sequence to that of the corresponding wildtype ABCB1, ABCB4, COMT, CRHR1, CRHBP, CYP3A4, DRD1, DRD2, DRD3, HRT1A, HTR1B, HTR2A, HTR3A, HTR3B, DRD3, MAOA, MAOB, SLC6A3, HTR2A, HTR2B, HTR2C, HTR3A, HTR3B, MAOA, MAOB, NR3C1, SLC6A2 SLC6A3, SLC6A4, TAC1, TACR1 or TPH genes; and c) training an algorithm to identify patterns of mutations in said humans which correlate with the response or non-response to anti-depressant medications, respectively.

7. The method according to claim 6, where training said algorithm on characteristic mutations according to claim 2, 3, or 4 comprises the steps of obtaining numerous examples of (i) said genomic mutational burden data, and (ii) historical clinical results corresponding to this genomic data;
constructing a algorithm suitable to map (i) said genomic mutational burden data as inputs to the algorithm to (ii) the historical clinical results as outputs of the algorithm;
exercising the constructed algorithm to so map (i) the said genomic mutational burden data as inputs to (ii) the historical clinical results as outputs; and
conducting an automated procedure to vary the mapping function, inputs to outputs, of the constructed and exercised algorithm in order that, by minimizing an error measure of the mapping function, a more optimal algorithm mapping architecture is realized;
wherein realization of the more optimal algorithm mapping architecture means that any irrelevant inputs are effectively excised, meaning that the more optimally mapping algorithm will substantially ignore input alleles and/or said genomic mutational burden data that is irrelevant to output clinical results; and
wherein realization of the more optimal algorithm mapping architecture, also known as feature selection, also means that any relevant inputs are effectively identified, making that the more optimally mapping algorithm will serve to identify, and use, those input alleles and/or genomic mutational burden data that is relevant, in combination, to output clinical results.

8. The method according to claim 6, where the algorithm is an algorithm using linear or nonlinear regression.

9. The method according to claim 6, where the algorithm is an algorithm using linear or nonlinear classification.

10. The method according to claim 6, where the algorithm is an algorithm using neural networks.

11. The method according to claim 6, where the algorithm is an algorithm using genetic algorithms.

12. The method according to claim 6, where the algorithm is an algorithm using support vector machines.

13. The method according to claim 6, where the algorithm is an algorithm using Bayesian probability functions.

14. The method according to claim 6, where the Bayesian probability functions algorithm is an algorithm using a Markov Blanket technique.

15. The method according to claim 6, where the algorithm is an algorithm using kernel based machines, such as kernel partial least squares, kernel matching pursuit, kernel fisher discriminate analysis, and kernel principal components analysis.

16. The method according to claim 6, where the algorithm is an algorithm using forward or backward selection methods such as forward floating search or backward floating search.

17. The method according to claim 7, where the feature selection algorithm is an algorithm according to one or more of claims 8, 9, 10, 11, 12, 13, 14, 15, or 16.

18. The method according to claim 7, where the feature selection algorithm is an algorithm using recursive feature elimination or entropy-based recursive feature elimination.

19. A method according to claim 6, wherein a tree algorithm, such as CART, MARS, or others, is trained to reproduce the performance of another machine-learning classifier or regressor by enumerating the input space of said classifier or regressor to form a plurality of training examples sufficient to span the input space of said classifier or regressor and train the tree to emulate the performance of said classifier or regressor.

20. The method according to claim 6, where the algorithm is a plurality of algorithms arranged in a committee network.

21. The method according to claim 5, 6, 7, 8, 9, 10, 11, 12, 13, 14, 15, 16, 17, 18, 19 or 20 where the anti-depressant medication belongs to the class known as Selective Serotonin Reuptake Inhibitors.

22. The method according to claim 5, 6, 7, 8, 9, 10, 11, 12, 13, 14, 15, 16, 17, 18, 19 or 20 where the anti-depressant medication is the molecule citalopram.

23. The method according to 5, 6, 7, 8, 9, 10, 11, 12, 13, 14, 15, 16, 17, 18, 19 or 20 where the anti-depressant medication is the molecule paroxetine.

24. The method of claim 2 wherein at least one mutation is a silent mutation, missense mutation, or combination thereof.

25. A method according to claim 1, wherein said sample is selected from the group consisting of a blood sample, a serum sample, a buccal swab sample, and a plasma sample.

26. A method according to any one of claim 1, 2, 3, 4, 5, 6, 7, 8, 9, 10, 11, 12, 13, 14, 15, 16, 17, 18, 19, 20, 21, 22, 23 or 24 wherein the presence of said mutation is detected by a technique that is selected from the group of techniques consisting of hybridization with oligonucleotide probes, a ligation reaction, a polymerase chain reaction and single nucleotide primer-guided extension assays, and variations thereof.

27. A method according to claim 2, wherein said correlating step comprises comparing said mutational burden to a second mutational burden measured in a second sample obtained from said patient, whereby, when said second mutational burden is of the type correlated by one or more of claims 5, 6, 7, 8, 9, 10, 11, 12, 13, 14, 15, 16, 17, 18, 19 or 20 than said second mutational burden, said patient is diagnosed as being responsive or resistant to SSRI anti-depressant therapy.

28. A method according to claim 2, wherein said second sample is obtained prior to treatment with an anti-depressant medication.

29. A method for detecting the presence or risk of developing depression in a human, said method comprising: determining the presence in a biological sample from a human of a nucleic acid sequence having a mutational burden according to claim 2 at one or more nucleotide positions in a sequence region corresponding to a wildtype genomic DNA sequence, wherein the mutational burden correlates with the presence of or risk of developing depression.

30. A method for evaluating a compound for use in diagnosis or treatment of depression, said method comprising: a) contacting a predetermined quantity of said compound with cultured cybrid cells or animal model having genomic DNA originating from an immortal neuronal rho or human embryonic kidney cell line and from tissue of a human having a disorder that is associated with severe depression and the mutational burden according to claim 2; b) measuring a phenotypic trait in said cybrid cells or animal model that correlates with the presence of said mutational burden and that is not present in cultured cybrid cells or animal model having genomic DNA originating from a neuronal rho cell line and genomic DNA originating from tissue of a human free of a disorder that is associated with severe depression; and c) correlating a change in the phenotypic trait with effectiveness of the compound.

31. A method according to claim 30 where the phenotypic trait is reuptake of serotonin, melanocortin, norepinephrine, dopamine or combinations of these.

32. A method according to claim 30 where the correlating step is according to one or more of claims 5, 6, 7, 8, 9, 10, 11, 12, 13, 14, 15, 16, 17, 18, 19 or 20.

33. A method for diagnosing treatment-resistant depression, said method comprising: determining the presence in a biological sample from a human of a nucleic acid sequence having a mutational burden according to claim 2, 3 or 4 at one or more nucleotide positions in a sequence region corresponding to a wildtype genomic DNA sequence, wherein the mutational burden correlates with the lack of response to SSRI depression medication.

34. A method according to claim 33 where the correlating step is according to one or more of claims 5, 6, 7, 8, 9, 10, 11, 12, 13, 14, 15, 16, 17, 18, 19 or 20.

35. A method according to claim 33, wherein said specific marker for treatment-resistant depression is selected from the group of genes consisting of ABCB1, ABCB4, COMT, CRHR1, CRHBP, CYP3A4, DRD1, DRD2, DRD3, HRT1A, HTR1B, HTR2A, HTR3A, HTR3B, DRD3, MAOA, MAOB, SLC6A3, HTR2A, HTR2B, HTR2C, HTR3A, HTR3B, MAOA, MAOB, NR3C1, SLC6A2 SLC6A3, SLC6A4, TAC1, TACR1 or TPH,

36. A therapeutic composition comprising antisense or small interfering RNA sequences which are specific to mutant genes according to claim 2, 3 or 4 or mutant messenger RNA transcribed therefrom, said antisense or small interfering RNA sequences adapted to bind to and inhibit transcription or translation of said target genes according to claim 2, 3 or 4 without preventing transcription or translation of wild-type genes of the same type.

37. The therapeutic composition of claim 36, wherein Depression is treated and wherein said mutant genes are selected from the group: ABCB1, ABCB4, COMT, CRHR1, CRHBP, CYP3A4, DRD1, DRD2, DRD3, HRT1A, HTR1B, HTR2A, HTR3A, HTR3B, DRD3, MAOA, MAOB, SLC6A3, HTR2A, HTR2B, HTR2C, HTR3A, HTR3B, MAOA, MAOB, NR3C1, SLC6A2 SLC6A3, SLC6A4, TAC1, TACR1 or TPH.

38. A kit comprising devices and reagents and a computer algorithm, compututing device and computational storage for measuring one or more mutational burdens of a patient and determining the diagnosis or prognosis in that patient for psychiatric illness.

39. The method of claim 38 when the mutational burden is that of claim 2, claim 3 or claim 4.

40. The method of claim 38 when the determination of diagnostic or prognostic outcome is made according to one or more of claims 5, 6, 7, 8, 9, 10, 11, 12, 13, 14, 15, 16, 17, 18, 19 or 20.

41. The method of claim 38 when the prognostic outcome is that of response to SSRI anti-depression medication.

42. The method of claim 41 when the determination of diagnostic or prognostic outcome is made according to one or more of claims 5, 6, 7, 8, 9, 10, 11, 12, 13, 14, 15, 16, 17, 18, 19 or 20.

43. The method of claim 38 when the diagnostic outcome is that of treatment-resistant depression.

44. The method of claim 43 when the determination of diagnostic or prognostic outcome is made according to one or more of claims 5, 6, 7, 8, 9, 10, 11, 12, 13, 14, 15, 16, 17, 18, 19 or 20.

45. The method of claim 38 when the prognostic outcome is that of response to the molecule citalopram.

46. The method of claim 45 when the determination of diagnostic or prognostic outcome is made according to one or more of claims 5, 6, 7, 8, 9, 10, 11, 12, 13, 14, 15, 16, 17, 18, 19 or 20.

47. The method of claim 38 when the prognostic outcome is that of response to the molecule paroxetine.

48. The method of claim 47 when the determination of diagnostic or prognostic outcome is made according to one or more of claims 5, 6, 7, 8, 9, 10, 11, 12, 13, 14, 15, 16, 17, 18, 19 or 20.

49. The method of claim 38 when the diagnostic outcome is that of determining risk of depression.

50. The method of claim 49 when the determination of diagnostic or prognostic outcome is made according to one or more of claims 5, 6, 7, 8, 9, 10, 11, 12, 13, 14, 15, 16, 17, 18, 19 or 20.

51. The method of claim 38 when the diagnostic outcome is that of determining risk of suicide.

52. The method of claim 51 when the determination of diagnostic or prognostic outcome is made according to one or more of claims 5, 6, 7, 8, 9, 10, 11, 12, 13, 14, 15, 16, 17, 18, 19 or 20.
